(19) United States
(12) Patent Application Publication
(10) Pub. No.: US 2003/0059811 A1
(43) Pub. Date: Mar. 27, 2003

(54) Methods of screening for ligands of target molecules

(76) Inventors: Hakim Djaballah, San Diego, CA (US); Scott Rongey, San Diego, CA (US); Rupal Patel, San Diego, CA (US); Mei Mei Wang, San Diego, CA (US); Joseph Coyle, San Diego, CA (US); Bin Li, San Diego, CA (US); Stephen Worland, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/170,758

(22) Filed: Jun. 13, 2002

Related U.S. Application Data
(60) Provisional application No. 60/298,531, filed on Jun. 14, 2001.
(60) Provisional application No. 60/356,315, filed on Feb. 13, 2002.

(51) Int. Cl. C12Q 1/68; G01N 33/53; G01N 33/542

(52) U.S. Cl. 435/6; 435/7.9

(57) ABSTRACT

The present invention provides methods of screening for ligands of target molecules. The methods of the present invention include assays in which a target molecule is subjected to denaturing conditions, and compounds are screened for the ability to alter the susceptibility of the target to unfolding. The methods of the present invention use fluorescence detection to determine that degree of unfolding of a target molecule. In some aspects of the present invention, fluorescence resonance energy transfer (FRET) is detected. In other aspects of the invention, fluorescence polarization (FP) is detected. In preferred embodiments, a target molecule such as a target protein is heated to a temperature, called T_{ATLAS}, at which at least a portion of the target molecule unfolds, in the presence of a test compound. In some embodiments of the present invention, the degree of unfolding of the target molecule is determined by binding of a specific binding member specific for the unfolded form of a target molecule that is coupled to a fluorophore that can participate in FRET. In some other embodiments of the present invention, the degree of unfolding of a target molecule is determined by FRET detection of aggregates of the target molecule. In yet other embodiments of the present invention, the degree of unfolding of a target molecule is determined by detection of fluorescence polarization of aggregates of the target molecule. The present invention provides sensitive, high throughput screens for identifying ligands of target molecules that are not dependent on the identity or function of the target.

### CROSS-REFERENCE TO RELATED APPLICATIONS

This application claims benefit of priority to U.S. Provisional Applications No. 60/298,531 filed Jun. 14, 2001 and No. 60/356,315 filed Feb. 13, 2002, both entitled “METHODS FOR IDENTIFYING COMPOUNDS THAT MODULATE PROTEIN FOLDING”, and both incorporated by reference herein.

### BACKGROUND OF THE INVENTION

The field of the invention relates to screening of compounds, such as screening for lead compounds that can be used for drug discovery. In particular, the present invention relates to high throughput screening methods for compounds that can bind a target molecule.

The drug discovery process relies on the screening of huge numbers of compounds to obtain lead compounds for drug development. Only a small fraction of the compounds tested for activity give a positive result, and only a small fraction of these “hits” will eventually lead to successful therapeutics. Many compounds identified by screens will not succeed as therapeutics due to unacceptable absorption, distribution, metabolism, excretion, or toxicity problems in animal studies or in clinical trials. Thus, there is a need to screen very large numbers of compounds against targets to obtain a sufficient number of hits to be able to develop safe and effective therapeutics.

The sequencing of the human genome and the sequencing of genomes of a number of infectious organisms has resulted in many new potential drug targets. Many of these potential drug targets that are known from bioinformatics as yet have no assigned function. Currently, a wide range of screening technologies are employed in the pharmaceutical and biotechnology industries to identify lead drug compounds, including cell-based assays, genetic screens, biophysical methodologies, and computer modeling (see for example, U.S. Pat. No. 5,876,946 issued Mar. 2, 1999 to Burbaum et al.; U.S. Pat. No. 6,242,190 issued Jun. 5, 2001 to Freire and Todd; U.S. Pat. No. 6,322,991 issued Nov. 27, 2001 to Pearlman et al.; U.S. Pat. No. 6,340,595 issued Jan. 22, 2002 to Vogels et al.; U.S. Pat. No. 6,373,577 issued Apr. 16, 2002 to Brauer et al). Many of these screens require knowledge of the function of the protein target.

Screens that identify compounds that bind a target molecule based on the ability of a ligand to affect the denaturation of a target molecule are also known in the art. For example, U.S. Pat. Nos. 6,376,180; 6,303,322; 6,232,085; 6,226,603; 6,036,920; 6,020,141; 5,585,277; 5,679,582; and 5,260,207; all herein incorporated by reference in their entireties, disclose assays that can rely on the ability of a ligand of a protein target to alter the susceptibility of the target to denaturation in response to denaturing conditions such as heat. As currently practiced, however, many assays that measure binding of test compounds to targets are lengthy, require multiple steps, and can have problems of compound and label interference.

There is a need for rapid, automatable screens that can be performed using small volumes and a minimum of steps and that can be used to screen compounds that can bind many different types of targets, including targets of unknown function, and that can result in the identification of compounds that have functional relevance.

### SUMMARY OF THE INVENTION

The present invention provides a set of related methods for identifying ligands of target molecules. The methods use fluorescence detection to determine the effect of test compounds on target unfolding in response to denaturing conditions such as heating.

One embodiment of the invention is a method of screening for ligands of a target protein that includes the use of a first specific binding member that specifically binds an unfolded form of the target protein. In this embodiment, the first specific binding member binds one member of a FRET pair, and a second specific binding member that can bind the other member of the FRET pair and can bind a different region of the target molecule is provided. The fluorescence signal depends on the interaction of the two FRET partners that are brought into proximity when the target molecule is denatured. Preferably, determination of the degree to which the target molecule is unfolded is determined by detection of fluorescence resonance energy transfer.

A second embodiment of the present invention also includes the use of a specific binding member that specifically binds an unfolded form of the target protein. In this embodiment, the specific binding member binds a fluorophore, and changes in FP are detected as the target unfolds in response to denaturing conditions.

A third embodiment of the present invention is a method of screening for ligands of a target protein that includes the use of a first specific binding member that can bind a FRET donor and a second specific binding member that can bind a FRET acceptor, where the first and second specific binding members bind the same single region of the target protein.

In one aspect of this embodiment, a portion of the population of target molecule is labeled with a first specific binding member, the target molecule population is subjected to denaturing conditions, and the second specific binding member is added to the assay sample. FRET is detected when the second specific binding member binds a target molecule that is aggregated with a target molecule that is bound to the first specific binding member. Thus, the FRET partners bound to the first and second specific binding members are brought into proximity by the unfolding and subsequent aggregation of target molecules that are bound by first specific binding members with target molecules that become bound by second specific binding members.

In another aspect of this embodiment of the present invention, one population of a target molecule is bound to a first specific binding member that binds one member of a FRET pair and a second population of the target molecule is bound to a second specific binding member that binds another member of the FRET pair. The first and second populations of target molecule are subjected to denaturing conditions and FRET is detected as denatured target molecules aggregate.

A fourth embodiment of the present invention is a method of screening for ligands of a target protein that includes the use of a fluorescent label that is attached to a target protein. Heating of the target protein results in changes in fluorescence polarization that occur as the protein unfolds and aggregates in solution. When the fluorophore-labeled target protein is heated in the presence of test compounds, those compounds that bind the target molecules and protect it against unfolding will have a reduced FP readout when compared with control samples that contain target molecule in the absence of test compound.

A fifth embodiment of the present invention is ligands identified using the methods of the present invention. The ligands can be formulated as therapeutic compounds in pharmaceutical compositions or optionally serve as the starting point for medicinal chemistry efforts to produce therapeutic compounds.

The present invention thus provides methods of screening for compounds using sensitive detection methods that can be configured as high throughput assays for ligands of a wide range of targets whose identity and function may be known or unknown.

### BRIEF DESCRIPTION OF THE FIGURES

FIG. 1 is a schematic representation of one aspect of a FRET assay configuration of the present invention. Two different antibodies that recognize the target are labeled with donor and acceptor fluorophores. One of the antibodies is specific for the unfolded target. When the target unfolds, a sandwich can be formed which positions the donor and acceptor fluorophore close enough to each other to undergo energy transfer.

FIG. 2 shows schematic representations of two aspects of FRET assay configurations of the present invention. In both aspects, at least a portion of a target molecule population is labeled with a member of a FRET pair using a first antibody, the target molecule population is heated, and a second antibody that recognizes the same region as the first antibody and that comprises a FRET partner is used to label the unlabeled portion of the target molecule population. Aggregates of target molecules are detected by FRET. In a) two mixtures of target protein are combined prior to heating; one of these two mixtures has the target protein labeled with a FRET partner. In b) the target protein is compared with a FRET partner labeled specific binding member prior to heating such that the step of pre-incubation of the first antibody with a protein of the protein is omitted.

FIG. 3 is a schematic representation of one aspect of a doped aggregation fluorescence polarization (FP) assay configuration. In this aspect of the present invention, a small percentage of a target molecule population is labeled with a fluorophore. After heating of the target molecule population, fluorescence polarization is detected. Increased levels of aggregation of the target molecule give an increased FP signal.

FIG. 4 depicts a CD thermal melting of target X90 giving a Tm of 43.3 degrees C. CD detection was carried out at various temperatures

FIG. 5 depicts the thermal melting of target X90 as a function of protein concentration in the unfolded-specific binding member TR-FRET assay format. Antibody concentrations were held constant for the titrations. TR-FRET detection was carried out at room temperature after samples were heated at various temperatures and cooled to room temperature.

FIG. 6 depicts a thermal melting of X90 in the unfolded-specific binding member TR-FRET assay format. The melting profile for 12 ng of target protein shows a midpoint transition of 47.3 degrees C.

FIG. 7 depicts a plot of the TR-FRET data from 3,840 control wells of target X90 at the screening temperature (T_{ATLAS}=53 degrees C.) and the low control temperature (4 degrees C.). The low control gives the signal in the absence of heat-induced target unfolding.

FIG. 8 depicts a thermal melt of target X90 in the presence of a known ligand (triangles). The control having no ligand present is also shown (diamonds); DMSO was added to controls at the same concentration as was present in ligand stock solution. In the presence of 10 micromolar ligand, the melting transition is pushed to higher temperature, indicating the ligand has conferred thermal protection to the target protein.

FIG. 9 is a scatter plot of the % inhibition of 7,744 compounds tested in duplicate against target X90. The degree of inhibition for each compound was plotted and the results from the two screens were plotted against each other. The diagonal line represents the ideal case where the compounds show exactly the same degree of inhibition in both screens.

FIG. 10 depicts titration curves for 20 independent compounds (“hits”) observed in duplicate assayed for binding to target X90 using the unfolded-specific binding member TR-FRET assay format. Each panel represents the titration of an independent compound.

FIG. 11 shows an ITC scan of duplicate hit compound A1 binding to target X90. The curve represents the fit to the data for a two binding site model. For this model, there is one tight binding site (K_{D}=0.5 micromolar), which agrees well with the IC50 of 1.5 micromolar obtained using the TR-FRET assay format (FIG. 10a). There is also a set of much weaker binding sites for the compound; an average of 4.6 compounds per target bind with an effective K_{D} of 50 micromolar.

FIG. 12 depicts a CD thermal melting of target DB7 giving a Tm of 50.3 degrees C. The melting profile shows that the protein undergoes irreversible unfolding as the temperature is increased.

FIG. 13 depicts a dynamic light scattering analysis of target DB7 used to assess aggregation upon unfolding. The increase in apparent molecular weight at higher temperatures indicates the unfolded target protein aggregated once it unfolded.

FIG. 14 depicts the thermal melting of target DB7 as a function of protein concentration in assay configurations in which. In a) two mixtures of target protein are combined prior to heating; one of these two mixtures has the target protein labeled with a FRET partner. In b) the target protein is compared with a FRET partner labeled specific binding member prior to heating such that the step of pre-incubation of the first antibody with a protein of the protein is omitted.

FIG. 15 depicts the thermal melting of target DB7 using the TR-FRET Configuration A format. The melting profiles are shown for 0, 3, 44, and 88 ng of target protein. The 44 and 88 ng conditions show a sufficiently large signal for determining the mid-point transition temperatures, giving Tm's of 47.5 degrees C. and 47.0 degrees C., respectively.

FIG. 16 is a plot of the data from 3,840 control wells for target DB7 at the screening temperature (T_{ATLAS}=49 degrees C.) and the low control temperature (4 degrees C.); the low control gives the signal in the absence of target unfolding.

FIG. 17 depicts a scatter plot of the % inhibition of 7744 compounds tested in duplicate against target DB7. The results of the two screens are plotted against each other. The diagonal line represents the ideal case in which the compounds show exactly the same degree of inhibition in both screens.

FIG. 18 depicts titrations of three compounds (hits) observed in duplicate screened against 10 micromolar target DB7. Percent inhibition was plotted as a function of concentration of test compound. Each panel represents the titration of an independent compound. The IC50 was calculated for each compound.

FIG. 19 depicts the CD spectra of target protein D56 at 4 degrees C.

FIG. 20 depicts a CD thermal melting of target D56 showing the protein undergoes irreversible unfolding as the temperature is increased.

FIG. 21 depicts a differential scanning calorimetry (DSC) profile for target D56. The protein undergoes two transitions (at approximately 45 degrees C. and approximately 53.5 degrees C.) as the temperature is increased.

FIG. 22 depicts the thermal melting of target D56 in the doped aggregation fluorescence polarization (DAFP) assay format. The concentration of the trace amount of labeled protein was held constant at 2 nanomolar and did not give an increased signal by itself at higher temperature. Increasing concentrations of unlabeled protein gave better signals at lower transition temperatures.

FIG. 23 is a plot of the average FP from the control wells for target D56 of each plate at the screening temperature (T_{ATLAS}=48 degrees C.) and the low control temperature (25 degrees C.); the low temperature control gives the FP value when no target unfolds.

FIG. 24 is a scatter plot in which the FP values from the duplicate screens of 4,933 compounds plotted against each other for each compound. The diagonal line represents the ideal case where the assay shows exactly the same FP value for a given compound in both screens.

FIG. 25 depicts titrations of eight duplicate hit compounds screened against 10 micromolar target D56. Percent inhibition is plotted as a function of concentration of test compound. The IC50 was calculated for each compound. Each panel represents an independent compound.

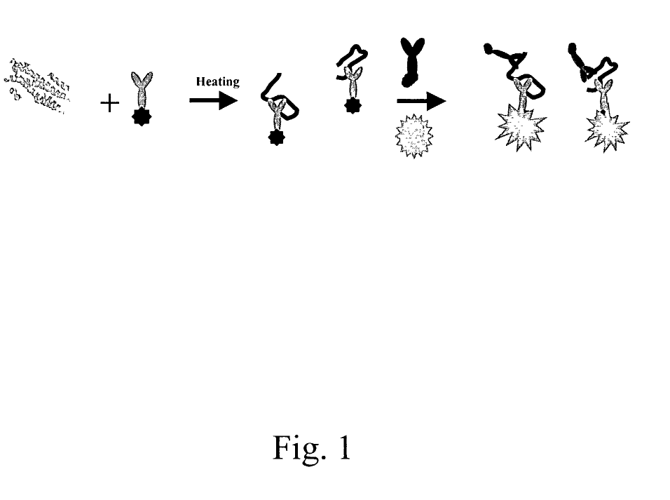

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down” or “upper” or “lower” and the like refer to orientation of parts during use of a device. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings:

A “target molecule” is a molecule of interest for which compounds that affect the structure or activity are desired.

A “target protein” is a protein for which compounds that affect the structure or activity are desired. A target protein can be a glycoprotein, lipoprotein, or nucleoprotein. A target protein can be a sulfated, glycosylated, phosphorylated, acylated, farnsylated, meristylated, or otherwise chemically or biochemically modified.

As used herein, a “linker” is a chemical structure that joins two molecules or moieties, such as, for example, a fluorophore and a target molecule. A linker that comprises active or activatable groups can be used to facilitate chemical linkage of two molecules or moieties. A linker can also provide spacing between the two molecules or moieties of interest such that they are able to function in their intended manner. Linkers can be chosen and designed based on such properties as, for example, their length, their flexibility, and their active or activatable groups. The coupling of linkers to molecules and moieties of interest can be through a variety of groups on the linker, for example, hydroxyl, aldehyde, amino, sulfhydryl, etc. Molecules and moieties can optionally be derivatized in a variety of ways for attachment to linkers. Coupling of linkers to molecules of interest, and moieties of interest can be accomplished through the use of coupling reagents that are known in the art.

As used herein, a “peptide linker” is a linker comprising a peptide sequence that joins two peptide or protein sequences, or a protein sequence with a peptide sequence. Preferably, a linker provides spacing between the peptides or proteins such that they are able to retain their biological or biochemical activity and function in their intended manner. For example, a linker can comprise a flexible peptide that separates a target protein from an attached peptide tag. In this way the target protein can be positioned at some distance from the peptide tag, such that, for example, the attached peptide tag does not interfere with a region of the target protein that may be involved with unfolding or aggregation. Linkers can be chosen and designed based on such properties as, for example, their length and their flexibility, or lack of stable secondary structure. Nonlimiting examples of linkers that can be useful in the present invention include, for example, peptide sequences that comprise hydrophilic amino acid residues and amino acid residues with short side chains, including those having with glycine, serine, and proline residues (see, for example Dubel et al. Gene 128: 97-101 (1993); Barbas et al. Proc. Natl. Acad. Sci. 88: 797807982 (1991); U.S. Pat. No. 5,258,498 issued Nov. 2, 1993 to Huston et al. and U.S. Pat. No. 5,908,626 issued Jun. 1, 1999 to Chang et al., all herein incorporated by reference).

When referring to binding, “directly” means that molecule A contacts and binds molecule B without intermediate molecules that mediate the binding and “indirectly” means that molecule A binds molecule B by contacting at least one intermediate molecule that mediates the binding.

A “test compound” is a chemical, compound, composition or extract to be tested by at least one method of the present invention for at least one activity such as specific binding capability. Test compounds can include small molecules, drugs, proteins or peptides or active fragments thereof, such as antibodies or fragments or active fragments thereof, nucleic acid molecules such as DNA, RNA or combinations thereof, or other organic or inorganic molecules, such as lipids, carbohydrates, or any combinations thereof. Test compounds, once identified, can be agonists, antagonists, partial agonists or inverse agonists of a target. Prior to performing an assay, a test compound is usually not known to bind to the target of interest.

“Substantially pure” refers to an object species or activity that is the predominant species or activity present (for example on a molar basis it is more abundant than any other individual species or activities in the composition) and preferably a substantially purified fraction or compound is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

A “specific binding member” is one of two molecules having an area on the surface or in a cavity which specifically binds to and is thereby defined as complementary with a particular spatial and polar organization of the other molecule. A specific binding member can be a member of an immunological pair (such as antigen-antibody), biotin-avidin, hormone-hormone receptor, nucleic acid duplexes, IgG-protein A, DNA-DNA, DNA-RNA, and the like.

As used herein, a “primary specific binding member” is a specific binding member that directly binds a target molecule, and a “secondary specific binding member” is a specific binding member that links a primary specific binding member to a fluorophore or a quencher.

An “unfolded-specific binding member” is a specific binding member that specifically binds the unfolded form of a target molecule and does not appreciably bind the native form of a target molecule.

“ATLAS” or “Any Target Ligand Affinity Screen” is not used herein as a Trademark, but to refer to assays such as those described herein.

A “fluorophore” is a molecule that, as a consequence of absorbing light at a particular wavelength, emits light of a characteristic wavelength spectrum. Fluorophores and methods of linkage of various fluorophores to different molecules for detection purposes are well known in chemistry and biochemistry. Many fluorophores useful in molecular detection are commercially available for example, from Molecular Probes (Eugene, Oreg.).

“Fluorescence resonance energy transfer” or “FRET” occurs when excitation energy is transferred between a donor fluorophore that has absorbed a photon and an acceptor moiety, causing quenching of donor fluorescence. If the acceptor moiety is a fluorophore whose excitation spectra overlap the emissions spectra of the donor, the acceptor moiety will fluoresce at its characteristic emissions wavelength. If the acceptor moiety is a not a fluorophore, it will quench fluorescence of the donor fluorophore without emitting light. In this case the acceptor moiety is a fluorescence quencher.

As used herein, a “donor fluorophore” is a fluorophore that, upon absorbing light, can transfer excitation energy to an acceptor fluorophore or a fluorescence quencher. This energy transfer can occur when the absorption spectrum of an acceptor fluorophore overlaps the emissions spectrum of the donor fluorophore. Other mechanisms also allow energy transfer when the acceptor is a quencher. In both cases, the light emitted by the donor fluorophore is quenched. However, if the excitation energy is transferred to an acceptor fluorophore, the acceptor fluorophore will fluoresce at its own characteristic emission wavelength spectrum, whereas if the energy is transferred to a quencher, there is no secondary fluorescence.

An “acceptor fluorophore” is a molecule that can accept excitation energy transferred by a donor fluorophore and use the transferred energy to emit light at its own characteristic emission wavelength spectrum.

A “FRET pair” consists of a donor fluorophore and an acceptor moiety, where the donor fluorophore, when exposed to light at its excitation wavelength, can transfer excitation energy to the acceptor moiety. This phenomenon, known as “fluorescence resonance energy transfer”, is dependent on the distance between donor and acceptor molecules and requires that the absorption spectrum of the acceptor overlaps the emissions spectrum of the donor. The two members of a FRET pair can be referred to as FRET partners.

A “FRET donor” is a donor fluorophore that can participate in FRET with another moiety herein referred to as a “FRET acceptor”. When a FRET donor and FRET acceptor are members of a FRET pair, and are positioned close enough to each other (determined by the Forster radius of the FRET pair), excitation energy can be transferred from the FRET donor to the FRET acceptor. After excitation by resonance energy transfer from the FRET donor, the FRET acceptor can fluoresce (in which case the FRET acceptor can be called an acceptor fluorophore) or not (in which case the FRET acceptor can be called a fluorescence quencher).

As used herein, a “fluorescence quencher” or “quencher” is a non-fluorescent molecule that can accept energy from an excited fluorophore, thereby reducing the fluorescence signal of the fluorophore.

“Fluorescence polarization” is a measure of the directionality of light emitted from a molecule after absorbing polarized light. Fluorescence polarization is defined by the following equation:

P=(I₍ₚₐᵣ₎−I₍ₚₑᵣ₎)/(I₍ₚₐᵣ₎+I₍ₚₑᵣ₎)

where P equals polarization, I₍ₚₐᵣ₎ equals the parallel component of emitted light, and I₍ₚₑᵣ₎ equals the perpendicular component of emitted light of a fluorophore when excited by plane polarized light, and the orientations “parallel” and “perpendicular” are relative to the excitatory light. P, the polarization unit, is independent of the fluorophore concentration and independent of the intensity of the emitted light. The relationship between fluorescence polarization and the limiting polarization, the fluorescence lifetime of the fluorophore, and the rotational relaxation time of the fluorophore is given by:

((1/P)−(1/3))=((1/P₀)−(1/3))×(1+(3 τ/ρ))

where ρ is the rotational relaxation time (ρ=3θ). FP decreases with shorter fluorescence lifetime, and increases with increasing rotational relaxation time, which in turn increases with molecular weight.

“Anisotropy” is a measurement of the directionality of light emitted from a molecule after absorbing polarized light. Anisotropy is defined by the following equation:

A=(I₍ₚₐᵣ₎−I₍ₚₑᵣ₎)/(I₍ₚₐᵣ₎+2I₍ₚₑᵣ₎)

where A equals anisotropy, I₍ₚₐᵣ₎ equals the parallel component of emitted light, and I₍ₚₑᵣ₎ equals the perpendicular component of emitted light of a fluorophore when excited by plane polarized light, and the orientations “parallel” and “perpendicular” are relative to the excitatory light.

“Plurality” means two or more. As used herein, “multiplicity” means more than two.

As used herein “denatured” means that a molecule has lost secondary, tertiary, or quaternary structure with respect to its native form. The terms “denatured” and “unfolded” are used interchangeably. A given molecule can exhibit degrees of denaturation or unfolding, and unfolding intermediates are also referred to as denatured or unfolded forms of the molecule.

The “native form”, “native conformation”, or “folded form” of a molecule refers to: 1) the structure of the molecule when the molecule is formed in nature, or 2) any active state of a molecule or fragment thereof. As the methods of the present invention are primarily concerned with biological molecules, the native conformation will usually refer to the conformation of the molecule found in or on a cell, virus, or tissue, or secreted by a cell or organism. However, the native conformation can also apply to the active or “native” form of a fragment of a biomolecule, where the native form is a form having the structure the fragment would have in the intact biomolecule, and can also apply to active forms of non-naturally occurring molecules (for example, chimeric molecules). The native conformation can refer to the native conformation of a processed or unprocessed molecule (such as a “pre”protein, “pro”protein, “pre” (unspliced) mRNA, etc.) and can refer to the conformation of a mutant or aberrant form protein or nucleic acid, for example, a form of a biomolecule found in a disease state.

“Tm” is midpoint temperature, and as used herein refers to the temperature at which half of a population of a molecule is in the unfolded state. In cases where a molecule undergoes more than one transition to an unfolded state, there may be more than one “transition temperature” at which half of the molecule has entered a particular “transition state” or intermediate unfolded state. A target molecule can exhibit a different Tm under different conditions (for example, salt concentration, surfactant concentration, etc., can have an effect on the Tm of a protein).

“T_{ATLAS}” refers to a temperature to which target molecules and test compounds are heated in screening methods of the present invention. T_{ATLAS} can be any temperature at which a change in the level of unfolded target molecule can be detected using the methods of the present invention. (A change in the level of unfolded target molecule can be detected by a direct or indirect determination of the amount or proportion of unfolded target molecules, by a direct or indirect determination of the amount or proportion of folded target molecules, or by a combination thereof.)

As used herein, “wells” can be any containers that can hold a liquid sample, and preferably containers that can hold small volume (sub-milliliter) liquid samples. For example, wells can be indentations of a surface, or can be capillaries or tubes for holding small volume liquid samples. In preferred aspects of the present invention, “wells” are wells of a multiwell plate.

A “reference value” is a measurement made using the methods of the present invention, or a calculated value from one or more measurements made using the methods of the present invention, that can be compared against assay measurements from test wells. Reference values can be measurements from, or calculations based on measurements from, control wells that comprise target protein in the absence of a test compound, or can be measurements from, or calculations based on measurements from, “standard” wells that comprise target protein and a compound. A compound in a standard well can be a compound whose affect on target unfolding is known or unknown. Measurements from more than one standard well comprising different compounds can be used to derive a reference value, such as, for example, an average measurement from a set of tested compounds.

In the present invention, an “attached tag” is any chemical or biochemical moiety that can be linked to a target molecule. Preferably, an attached tag is a moiety that can be specifically bound by one or more specific binding members, including specific binding members that comprise or bind fluorophores. Preferably, an attached tag is covalently linked to a target molecule. An attached tag can be a chemical moiety such as DNP or biotin that can be chemically coupled to a target molecule. An attached tag can also be a peptide. In aspects where the target molecule is a protein, an attached tag can be an engineered peptide tag, and can optionally be attached to the protein by incorporating the peptide tag sequence into the protein sequence using recombinant DNA technology. A “single attached tag” is a tag that occurs only once on a particular target molecule. Similarly, a “single peptide tag” is a peptide sequence that occurs only once in a particular target protein sequence. The use of the terms “single attached tag” and “single peptide tag” is intended to mean that in the present invention a particular tag does not occur more than once in or on a target molecule. The use of the terms “single peptide tag” and “single attached tag” does allow for the occurrence of one or more additional tags on the same target protein, as long as the one or more additional tags have a distinct chemical identity from the “single peptide tag” or “single attached tag”, and are not bound by the same specific binding members.

Introduction

The present invention recognizes the need to provide a large number of lead compounds in the drug discovery effort. The present invention provides high throughput screening methods that can be used to efficiently screen a wide variety of target types in a short time period, using multiple small volume samples and high sensitivity/low background fluorescence detection methods. The assays of the present invention include the use of generic labeling reagents that result in a minimum of detection interference, the elimination of wash steps, minimal incubations, and rapid detection.

The present invention provides assay methods that determine the degree of unfolding of a target molecule in the presence and absence of a test compound. A difference in the degree of unfolding of a target molecule in the presence of a test compound with respect to controls is indicative of the ability of a test compound to bind and thereby alter the stability of the target molecule during heating. Thus, test compounds that alter the degree of unfolding of a target molecule can be identified as ligands of the target molecule.

The methods of the present invention rely on fluorescence readouts as indicators of the degree of unfolding of a molecule. In particular, the assays use fluorescence resonance energy transfer (FRET) detection or fluorescence polarization (FP) detection and specifically labeled target molecules to measure target molecule unfolding. Fluorescence spectroscopy, including FRET and FP spectroscopy, is well known in the art and discussed in Principles of Fluorescence Spectroscopy, 2^{nd} edition (1999) ed. by Joseph R. Lakowicz, Plenum Publishing Corp. The advantages of using FRET and FP in the methods disclosed herein include the stability of the labeling reagents (fluorophores), high sensitivity, very rapid detection, and the capacity to automate detection.

In some preferred embodiments of the present invention, FRET detection is employed. In these embodiments, two specific binding members that can bind a target molecule are used, each of which binds a member of a FRET pair. When a fluorophore is exposed to a certain wavelength of light, it emits light (fluoresces) at a different wavelength. However, during FRET, a fluorophore that is stimulated by light can nonradiatively transfer excitation energy to an acceptor moiety. This causes quenching of the fluorescence of the donor. If the excited state energy is transferred to another fluorophore, the acceptor fluorophore will fluoresce at its own characteristic emissions wavelength spectrum. If, on the other hand, the excited state energy is transferred to a non-fluorophore acceptor, the fluorescence of the donor will be quenched without fluorescence emission by the acceptor. Pairs of molecules that can engage in FRET are called FRET pairs. For FRET to occur, the members of the FRET pair (the FRET partners) must be in close proximity and the excitation spectra of the donor must overlap the emissions spectra of the acceptor (Clegg et al. (1992) Methods in Enzymology 211: 353-388; Selvin (1995) Methods in Enzymology 246: 300-334).

Fluorescence polarization is another highly sensitive means of detection used in the present invention. Fluorescence polarization refers to the propensity of a fluorescent molecule to emit light in the same direction in which it is absorbed. However, if the fluorophore is rotating in solution during the lifetime of fluorescence emission, the emitted light will be less polarized than the excitation light. Any conditions that slow the rotation of a fluorophore will increase the directionality of emitted light and thus increase the degree of polarization of fluorescence emission. This phenomenon can therefore be used to investigate and quantitate phenomena that slow the rotation of molecules in solution, such as binding to a stabilized moiety, undergoing an increase in size, increasing the viscosity of the solution, etc.

In some preferred methods of the present invention, fluorescence detection methods are used to detect soluble aggregates of the target molecule. In these assays, a target molecule is subjected to denaturing conditions in the presence of a test compound. As the target molecule unfolds, it tends to form aggregates with other target molecules in solution. Although the present invention is not limited to any particular mechanism, it is likely that unfolding of a target molecule exposes regions of a molecule that are not otherwise exposed in solution, and that these regions can participate in intermolecular binding, leading to soluble aggregates of the target molecule. When a target molecule is labeled with a fluorophore, these soluble aggregates can be detected by their reduced rate of rotation using FP detection. When different members of the target molecule population of target molecules are labeled with FRET donors and FRET acceptors, aggregates of target molecules can be detected by the proximity of FRET partners on aggregated target molecules by measuring donor emission, acceptor emission, or a combination thereof. Test compounds that bind target molecules and alter the stability of the target molecule under denaturing conditions will also affect the aggregation of target molecules and therefore alter the fluorescence readout (using FP or FRET detection) with respect to controls.

The present invention thus has features that provide for rapid, small volume screening for ligands that produces very low background. The signal is dependent on the amount of target unfolding, and the fluorescence readout reports directly on the unfolded state (in many cases, the aggregated state) of the protein. There is a greatly reduced potential for artifacts that can occur in assays having labeled test compounds or free label molecules.

Thus the present invention provides a variety of easily set up, rapid, highly reproducible, high signal-to-noise assays that are based on the ability of a ligand to stabilize or destabilize the secondary structure of a target molecule and influence the degree of unfolding and aggregation of the target molecule when heated to a predetermined temperature. The assays rely on fluorescence detection, where the fluorescence signal is directly related to the degree of unfolding of the target molecule, such that the signal is rapidly detected with minimal background.

A first embodiment of the invention is a method of screening for ligands of a target protein that includes the use of a first specific binding member that specifically binds a denatured form of the target protein. In this aspect of the invention, the first specific binding member binds one member of a FRET pair, and a second specific binding member that can bind the other member of a FRET pair is also included in the assay, such that the fluorescence signal depends on the interaction of the two FRET partners that are brought into proximity as the target molecule is denatured. Preferably, determination of the degree to which the target molecule is unfolded is determined by detection of fluorescence resonance energy transfer.

A second embodiment of the present invention also includes the use of a specific binding member that specifically binds an unfolded form of the target protein. In this embodiment, the specific binding member binds a fluorophore, and changes in FP are detected as the target unfolds in response to denaturing conditions.

A third embodiment of the present invention is a method of screening for ligands of a target protein that includes the use of a first specific binding member that can bind a FRET donor and a second specific binding member that can bind a FRET acceptor, where the first and second specific binding members bind the same single region of the target protein.

In one aspect of this embodiment, a portion of the population of target molecule is labeled with a first specific binding member, the target molecule population is subjected to denaturing conditions, and the second specific binding member is added to the assay sample. FRET is detected when the second specific binding member binds a target molecule that is aggregated with a target molecule that is bound to the first specific binding member. Thus, the FRET partners bound to the first and second specific binding members are brought into proximity by the unfolding and subsequent aggregation of target molecules that are bound by first specific binding members with target molecules that become bound by second specific binding members.

In another aspect of this embodiment of the present invention, one population of a target molecule is bound to a first specific binding member that binds one member of a FRET pair and a second population of the target molecule is bound to a second specific binding member that binds another member of the FRET pair. The first and second populations of target molecule are subjected to denaturing conditions and FRET is detected as denatured target molecules aggregate.

A fourth embodiment of the present invention is a method of screening for ligands of a target protein that includes the use of a fluorescent label that is attached to a target protein. Heating of the target protein results in changes in fluorescence polarization that occur as the protein unfolds and aggregates in solution. When the fluorophore-labeled target protein is heated in the presence of test compounds, those compounds that bind the target molecules and protect it against unfolding will have a reduced FP readout when compared with control samples that contain target molecule in the absence of test compound.

Elements of the Invention

Target Molecules

Target molecules used in the methods of the present invention can be molecules of any type, but preferably target molecules are molecules that have secondary, tertiary, or quaternary structure that can be altered by heating. For example, target molecules can comprise large organic molecules, carbohydrates, proteins, lipids, nucleic acids, or combinations thereof. Preferred target molecules are target molecules that comprise peptides, proteins, or nucleic acids.

In some preferred aspects of the present invention, a target molecule comprises one or more proteins, and can also optionally include other moieties, including organic molecules and inorganic molecules, such as cofactors, prosthetic groups, lipids, carbohydrates, nucleic acids, etc. A target molecule can be a monomeric, dimeric, or oligomeric form of a protein. A target molecule can also be a complex of more than one protein, where one or more of the proteins in the complex can comprise one or more other moieties.

A target protein used in the methods of the present invention can be from any source, such as isolation from cells or media, including cells that are genetically engineered to synthesize the target protein. Genetically engineered cells can be from any species, including, as nonlimiting examples, bacterial species, fungal species, insect species, avian species, and mammalian species. A target protein can be a protein that has been modified by the introduction of one or more mutations into the nucleic acid molecule that encodes it, where a mutation can be any mutation, including one or more deletions, insertions, truncations, substitutions, or combinations thereof. A target protein can include one or more domains of other proteins, and can be a fusion protein that incorporates regions from two or more proteins. A target protein can also be chemically or enzymatically modified, and can comprises moieties such as, but not limited to, active groups, labels, or specific binding members.

Attached Tags

Target molecules of the present invention can optionally comprise attached tags. Attached tags are chemical or biochemical moieties that are linked to a target molecule. Preferably, attached tags are covalently bound to a target molecule. Optionally, attachment of a tag to a target molecule can be via a chemical linker. In the methods of the present invention, attached tags are used as binding sites for specific binding members, such as specific binding members that can directly or indirectly bind fluorophores or quenchers. The use of attached tags has several advantages, including the ability to use specific binding members that bind a target molecule without binding endogenous regions of a target molecule that may participate in folding/unfolding or ligand binding. The use of attached tags can also allow for the use of generic reagents in assays of the present invention, such as specific binding members that recognize the attached tags and comprise or bind fluorophores or quenchers.

Attached tags can be chemical moieties that can be chemically or enzymatically coupled to a target molecule. Nonlimiting examples of such attached tags are dinitrophenyl (DNP) and biotin. Attached tags can also be peptide sequences. Where the target molecule is a protein, peptide sequences that can be recognized by specific binding members can be incorporated into the open reading frame of a gene encoding the target protein using genetic engineering. Target proteins comprising peptide tags can be produced by transformed prokaryotic or eukaryotic cells.

Several peptide tags are known in the art and antibodies that specifically bind to them are commercially available. However, the present invention is not limited to known peptide tags. For example, novel peptide tags can be adopted or developed for use in the present invention.

Peptide tags are preferably attached to the C or N terminus of a target protein to minimize interference with native conformation of the target protein. They can optionally be attached using linkers, such as, but not limited to, peptide linkers.

Test Compounds

Test compounds used in the methods of the present invention can be any compounds, including but not limited to, small molecules, organic or inorganic compounds, including but not limited to carbohydrates, saccharides, peptides, proteins, lipids, sterols, nucleic acids, and combinations thereof.

Test compounds can be from compound libraries that can be generated in any of a variety of ways. For example, combinatorial chemistry, phage display, or ribosome display can be used to generate compounds that can be assayed using the methods of the present invention. Compounds can be synthesized and selected for testing in assays based on rational drug design, including the use of computer programs that can use information on target protein structure and homology and optionally, criteria for solubility, low likelihood of toxicity, manufacturability, etc.

The compound libraries can be targeted or untargeted, and can be subsets, or expanded sets, of other libraries. Compounds that have demonstrated interaction with a target molecule in assays of the present invention or other assays can be used as a basis for testing or designing similar compounds. For example, a chemical skeleton structure can be based on an assay hit or on a known compound, and the skeleton can be elaborated randomly or nonrandomly to generate further test compounds for assays of the present invention.

Test compounds can also be mixtures of compounds that can be fractions or extracts of plants, fungi, bacteria, marine organisms, or growth media. The fractions, extracts, or media of organisms can be further fractionated, partially or substantially purified.

Test compounds can be made up in solutions that comprise one or more buffers, salts, reducing agents, chelators, surfactants, alcohols, glycerol, DMSO, etc. Preferably the test compound solutions are made up such that the solution, when added to the assay mixture, is compatible with the assay.

Specific Binding Members

Specific binding members used in the present invention can include any specific binding members, including antibodies, proteins, peptides, small molecules, and nucleic acids. In some aspects of the present invention, specific binding members are used that specifically bind a target molecule. Specific binding members that bind a target molecule can be any specific binding members that specifically bind the target molecule, including an attached tag of a target molecule. Preferred specific binding members are antibodies and biotin/streptavidin.

In the present invention, specific binding members are used, as nonlimiting examples, to bind a fluorophore or quencher to a target molecule, or to bind a fluorophore or quencher to another specific binding member to a target molecule. Fluorophores or quenchers bound to specific binding members can be chemically coupled to specific binding members, or bound through secondary specific binding members. “Primary specific binding members” directly link a fluorophore or quencher to target molecule, thus, they are generally chemically coupled to a fluorophore or quencher. “Secondary specific binding members” indirectly link a fluorophore or quencher to target molecule, thus, they are generally chemically coupled to a fluorophore or quencher and can bind a primary specific binding member.

Preferred specific binding members used to directly bind a target protein include antibodies, particularly monoclonal antibodies. Antibody fragments, such as but not limited to Fab fragments, that retain the specific binding activity of the antibody molecule can also be used as specific binding members in the methods of the present invention.

In some configurations of the assays of the present invention, one or more specific binding members are added prior to the heating of a sample. In such cases, the binding of the specific binding members should not be reduced by the temperatures used in the assay. Antibodies developed or purchased for use in the methods of the present invention that are present during the heating of a sample can be tested for the stability of binding during heating to assay temperatures. In some cases where binding of an antibody is heat-sensitive, it may be possible to reconfigure the assay such that binding of that particular antibody is added after heating and subsequent cooling to a binding-compatible temperature (such as room temperature).

Fluorophores

The present invention uses fluorescent labels that can be directly or indirectly bound to a target molecule. Fluorescent molecules or fluorophores are well known in the art, as are methods of binding fluorophores to other molecules, for example, by coupling through active groups. Fluorophores can also be indirectly bound to a target molecule, for example, through binding of a specific binding member that is coupled to a fluorophore.

In some methods of the present invention, fluorescence polarization is detected. Fluorophores that can be directly or indirectly bound to a target molecule for fluorescence polarization detection include any fluorophores known in the art or later developed, for example, fluorescein, rhodamine, Alexa dyes, Cy dyes, TMR, JOE, FAM, TAMRA, BODPY, pyrene, europium or other lanthanide compounds, and fluorescent proteins such as phycoerythrin, phycocyanin, allophycocyanin, GFP and its derivatives, D.s. red, etc. In some other methods of the present invention, fluorescence resonance energy transfer is detected. In these methods, a fluorescence donor/acceptor pair is used. Any donor/acceptor fluorophore pair in which the donor fluorophore that can absorb light and transfer excitation energy to the acceptor fluorophore, causing the acceptor fluorophore to fluoresce, can be used. Examples of donor/acceptor pairs useful in the methods of the present invention include: terbium/fluorescein, terbium/GFP, terbium/TMR, terbium/Cy3, terbium/R phycoerythrin, Europium/Cy5, Europium/APC, Alexa 488/Alexa 555, Alexa 568/Alexa 647, Alexa 594/Alexa 647, Alexa 647/Alexa 594, Cy3/Cy5, BODIPY FL/BODIPY FL, Fluorescein/TMR, IEDANS/fluorescein, and fluorescein/fluorescein.

A fluorophore or quencher that can be directly or indirectly bound to the first specific binding member can be any fluorophore or quencher that, together with a quencher or fluorophore directly or indirectly bound by a second specific binding member, constitutes a FRET pair. By “donor fluorophore” is meant that when activated by light, the fluorophore can transfer excitation energy to an acceptor fluorophore. By “acceptor fluorophore” is meant that the fluorophore will accept excitation energy from a donor fluorophore that is excited by light of an appropriated wavelength. Nonlimiting examples of donor fluorophores that can be useful in the methods of the present invention include terbium, Alexa 488 , Alexa 568, Alexa 594, Alexa 647, Cy3, BODIPY FL, fluorescein, IEDANS, EDANS, or Europium compounds. Nonlimiting examples of acceptor fluorophores that can be useful in the methods of the present invention include fluorescein, GFP, TMR, Cy3, R phycoerythrin, Cy5, APC, Alexa 555, Alexa 647, Alexa 647, Alexa 594, Cy5, BODIPY FL, TMR, XL-665, and allophycocyanin.

In addition, it is also possible to use a fluorophore that can be quenched by a fluorescence quencher bound to a second specific binding member, or the first specific binding member can comprise or bind a quencher and the second specific binding member used in the assay can bind a donor fluorophore. Nonlimiting examples of fluorescence quenchers that can be used in the methods of the present invention include DABCYL, DABSYL, QSY 7, QSY 9, QSY 21, and QSY 35.

Heating of Samples

In some preferred methods of the present invention, samples that comprise a target molecule and one or more test compounds are heated to one or more predetermined temperatures to determine the effect of a test compound on target molecule unfolding. In these methods, the temperature to which a sample is heated during the assay is called T_{ATLAS}. (If a sample is heated to more than one temperature, the temperatures can be called T_{ATLAS1}, T_{ATLAS2}, T_{ATLAS3}, etc.). T_{ATLAS} can be any preselected temperature at which a measurable amount of target molecule unfolds under given conditions, where the amount of target molecule that unfolds can be determined by a direct or indirect measurement of the amount of unfolded target molecule in a sample, by a direct or indirect measurement of the amount of folded target molecule in a sample, or a combination thereof.

Characterization of a target molecule to determine a temperature at which a measurable amount of target molecule unfolds can be done by heating the molecule in increments or at a defined rate while monitoring the structure of the molecule, for example, using differential scanning calorimetry (DSC), circular dichroism (CD), nuclear magnetic resonance (NMR), UV absorption spectroscopy, fluorescence (including fluorescence emission and fluorescence polarization), light scattering, or any other method that can be used to reveal the structure of a molecule. Preferably, characterization of a target molecule such as a target protein includes determination of the target molecule's midpoint temperature (Tm), but this is not a requirement of the present invention.

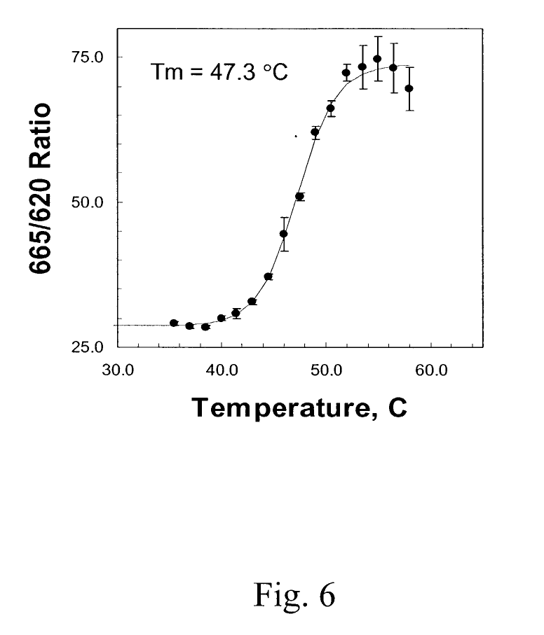

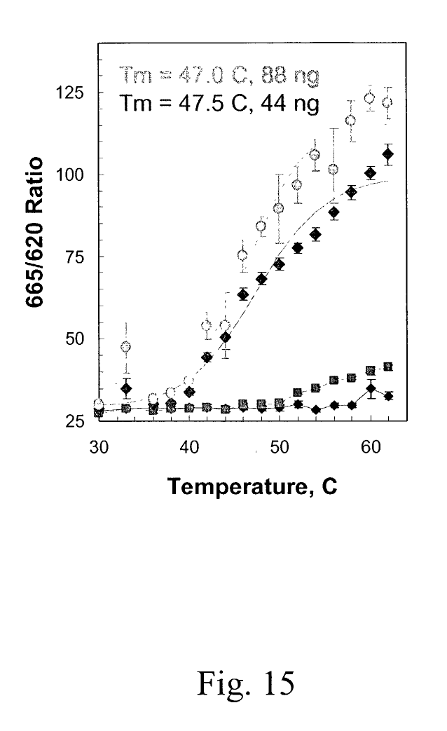

Preferably, the target molecule is also subjected to structural determinations at a series of temperatures using labeling and detection systems configured in the same way as the ATLAS assay that will be used to screen for ligands. See for example, Example 2 and FIG. 6; Example 8 and FIG. 15. This allows the practitioner to plot the relationship between temperature and target unfolding under assay conditions. T_{ATLAS} can be selected using the relationship between temperature and target unfolding under assay conditions. Criteria that can be used for the selection of T_{ATLAS} are the dynamic range, or the potential for measuring large changes in the degree of unfolding at various temperatures, the assay quality or “robustness” (Z′ factor) at various temperatures (which depends on the signal-to-noise ratio obtained in the assay and the precision of the assay); the sensitivity of the assay at various temperatures, and the stability of assay reagents at various temperatures.

The heating of a target molecule to T_{ATLAS} in an assay of the present invention can be essentially continuous, or it can occur in discrete steps. Preferably heating is at a defined rate and relatively rapid, for example, 0.5 degrees per minute or faster. However, the rate of heating is not a limitation of the present invention.

Once heated to the predetermined temperature, test wells can be held at that temperature for any length of time. Preferably, test wells are incubated at T_{ATLAS} from about one minute to about six hours, and more preferably from about ten minutes to about one hour.

After heating and optional incubation at the predetermined temperature, test wells can optionally be cooled to a lower temperature. It is convenient to cool the samples to about room temperature (about 22 degrees C.) prior to performing fluorescence detection. However, samples can be cooled to other temperatures, such as temperatures below 37 degrees C., prior to fluorescence detection. It is also within the scope of the present invention to maintain the test wells at T_{ATLAS} during fluorescence detection.

In preferred aspects of the present invention, test wells are heated to a single discrete temperature and after a specified length of time at the single temperature, the wells are cooled to room temperature and measurements of fluorescence emission or fluorescence polarization are made.

Measurement of Fluorescence

The assays of the present invention use fluorescence detection to determine the unfolded state of a target molecule. In the methods of the present invention, fluorescence detection can be detection of fluorescence resonance energy transfer (FRET) or detection of fluorescence polarization (FP).

Where FRET detection is employed, target molecules are directly or indirectly labeled with FRET donors and FRET acceptors. When the FRET partners are brought into proximity by protein unfolding, fluorescence resonance energy transfer is detected. For example, one portion of a target molecule population can be labeled with a FRET donor, and another portion of a target molecule population can be labeled with a FRET acceptor.

If the FRET pair used in the assay comprises a donor fluorophore and a fluorescence quencher, detection is at a wavelength of the donor emissions spectrum. The fluorescence readout will be reduced with increased target molecule unfolding which brings the FRET partners into proximity with one another.

If the FRET pair used in the assay comprises a donor fluorophore and an acceptor fluorophore, detection can be at a wavelength of the donor emissions spectrum, a wavelength of the acceptor emissions spectrum, or, preferably, both. The ratio of acceptor emission to donor emission can be used as a basis for comparing test wells comprising test compounds with control test wells. The fluorescence readout from the donor fluorophore will be reduced with increased target molecule unfolding which brings the FRET partners into proximity with one another. The fluorescence readout from the acceptor fluorophore will be increased with increased unfolding of the target molecule which allow the FRET partners come into proximity with one another. The ratio of acceptor to donor emission will therefore also increase with increased target unfolding.

Preferably, FRET-based emission measurements are time-resolved, but this is not a requirement of the present invention. Measurement of time-resolved fluorescence resonance energy transfer can reduce the interference from background fluorescence, for example, from the wells that contain the samples.

Where FP detection is employed, preferably a fraction of the target molecules or a target molecule population are directly or indirectly labeled with fluorescent labels, although it is also possible to detect FP from the intrinsic fluorescence of a target molecule. Unfolding of a target molecule, and/or unfolded target molecules such as proteins due to altered hydrodynamics of unfolded vs. folded proteins, can result in aggregates that have a reduced rate of rotation in solution with respect to unaggregated target due to their increased size. The reduced rate of rotation results in increased polarization of the light emitted by the fluorophore when compared with non-aggregated target protein. In assays of the present invention, comparison of FP values of test wells comprising test compounds with FP values of control test wells after heating of the test wells to T_{ATLAS} can be used to determine the degree of unfolding of the target molecule in the presence of a test compound.

It is also possible to measure an increase in FP of a target molecule that occurs because of binding of an unfolded-specific binding member. Binding of an unfolded-specific binding member can reduce the rate of rotation of the target molecule to give a measurable increase in FP. Moieties such as large molecules or particles can optionally be attached to an unfolded-specific binding member to increase the size of the unfolded target/an unfolded-specific binding member complex and increase the FP signal. It is also possible to label the target with a fluorophore, and to measure a decrease or increase in the FP signal as the target molecule unfolds and is able to rotate more or less freely.

Reference Values

In the methods of the present invention, measurements of test wells can be compared with reference values to determine whether a test compound alters the stability of a target protein under denaturing conditions.

Reference values can be measurements made from control wells, where a control well comprises a target molecule and lacks a test compound, and is treated in the same way as a test well (addition of reagents, incubations, etc.). One or more control wells can be assayed at a time different from the time the one or more test wells are assayed, but preferably a control well is assayed at the same time as a test well. If one or more control wells is assayed at a time different from the time the one or more test wells are assayed measurements made from the control well or wells can be recorded and results of assays with test compounds can be compared with the stored data.

Reference values can also be measurements made from one or more standard wells, where a standard well comprises a target molecule and one or more compounds that may or may not affect the stability of a target molecule, and the standard well or wells is treated in the same way as a test well (addition of reagents, incubations, etc.). A standard well can comprise a test compound (whose effect on the target molecule is being tested), or a compound whose effect on the stability of the target molecule under denaturing conditions is known. One or more standard wells can be assayed at a time different from the time the one or more test wells are assayed, but preferably a standard well is assayed at the same time as a test well. If one or more standard wells is assayed at a time different from the time the one or more test wells are assayed, measurements made from the standard well or wells can be recorded and results of assays with test compounds can be compared with the stored data.

Reference values include not only measurements made from one or more control wells or one or more standard wells, but also values derived therefrom. For example, a reference value used in the methods of the present invention can be an average of measurements of two or more standard wells that comprises the same compound or different compounds, an average of two or more control well measurements, ratios (or averages of ratios) derived from measurements of control or standard wells (for example, acceptor to donor wavelength fluorescence intensity ratios), or the result of any practical manipulation of measurements made on control wells, standard wells, or a combination of control wells and standard wells.

I. Methods of Screening to Identify One or more Compounds that Bind to a Target Molecule Using a Specific Binding Member that Recognizes the Unfolded Form of a Target Molecule and FRET Detection

One embodiment of the present invention is a screening method for identifying one or more ligands of a target molecule in which the screening method uses a specific binding member that specifically recognizes the unfolded form of a target molecule and FRET detection. By “specifically recognizes” is meant that the specific binding member binds target molecules that are in the unfolded state, but does not appreciably bind target molecules that are in the folded, or native, state. The target molecule is contacted with at least one test compound and subjected to a denaturing treatment in the presence of a specific binding member specific for the unfolded form of the target molecule (hereinafter referred to as an “unfolded-specific binding member”). As the protein unfolds, the unfolded-specific binding member binds to unfolded target molecules. After the denaturation step, a second specific binding member is added. The second specific binding member specifically binds a region of the target molecule that is distinct from the region that is bound by the unfolded-specific binding member. The first and second specific binding members each comprise or bind a member of a fluorescence resonance energy transfer (FRET) pair. That is, one of the specific binding members binds a FRET donor and the other specific binding member binds a FRET acceptor. Thus when the FRET partners are in proximity, such as when they bind the same target molecule after unfolding of the target molecule or when they bind components of an aggregate formed after unfolding, energy can be transferred from a FRET donor to a FRET acceptor. One or more fluorescence signals is detected, and when compared with fluorescence measurements of a control (in which target molecule is at least partially denatured in the absence of a test compound) or standard (in which target molecule is at least partially denatured in the presence of a test compound), the fluorescence measurement is use as an indicator of the degree to which the target molecule occurs in the unfolded or folded state under the denaturing condition. Test compounds that alter the degree to which the target molecule occurs in the unfolded state at the assay temperature are identified as ligands of a target protein. In preferred embodiments, the denaturing treatment is heating to one or more predetermined assay temperatures (at which the target molecule is known to unfold to a measurable extent in the absence of a test compound).

The method includes: providing a target molecule in solution in one or more test wells; adding to the one or more test wells one or more test compounds; adding to the one or more test wells a first specific binding member that specifically binds the unfolded form of the target molecule, where the first specific binding member comprises a FRET donor or acceptor, or can directly or indirectly bind a FRET donor or a FRET acceptor; and subjecting the, one or more test:, wells to conditions at which at least a portion of the target molecule is denatured. The method further includes adding to the one or more test wells a second specific binding member that can bind said target molecule at a site distinct from the binding site of the first specific binding member. The second specific binding member comprises or can bind a FRET donor or a FRET acceptor, depending on the nature of the fluorophore attached to or integral to the first specific binding member, such that when the first specific binding member comprises or can directly or indirectly bind a FRET donor, the second specific binding member comprises or can directly or indirectly bind a FRET acceptor, and when the first specific binding member comprises or can directly or indirectly bind a FRET acceptor, the second specific binding member comprises or can directly or indirectly bind a FRET donor. The method further includes measuring fluorescence emission at one or more wavelengths from the one or more test wells; making a comparison of fluorescence emission at one or more wavelengths of one or more test wells with a reference value; using said comparison of fluorescence emission to determine the extent to which the target molecule occurs in the unfolded state, the folded state, or both, in the wells comprising target molecules and test compounds; and using the determination of the extent to which said target molecule occurs in the unfolded state, the folded state, or both, in the wells comprising target molecule and test compounds to determine whether one or more test compounds binds said target molecule, thereby identifying one or more ligands of the target molecule.

The target molecule for which ligands are sought can be any molecule, but preferably the target molecule is a biomolecule, more preferably a biomolecule that comprises a peptide, a protein or a nucleic acid, and most preferably a biomolecule that comprises a protein. A biomolecule that comprises a protein or peptide can be, for example, a protein that comprises chemical or post-translational modifications, and can be for example, a glycoprotein, nucleoprotein, lipoprotein, farnysylated, myristylated, acylated, phosphorylated, or sulfated protein, etc. Where “protein” or “target protein” is used herein, the aforementioned biomolecules that comprise protein are also included. It can also include peptide or chemical moieties such as but not limited to linkers, labels (including fluorophores), tags, and specific binding members.

Target proteins can be of any species origin and can be isolated from native sources, including organisms, environmental sources, or media, or can be produced using recombinant technologies using endogenous or exogenous cell types. For example, target proteins can be produced in bacterial or fungal cultures, insect cell cultures, avian cell cultures, mammalian (including human) cell cultures, etc. They can also be produced by transgenic organisms. The proteins are preferably at least partially purified, and more preferably substantially purified, for use in assays. The proteins can differ in sequence with regard to the native wild-type form, and can include one or more attached tags.

In preferred aspects of the present invention, a target protein can include an attached tag that can be recognized by a specific binding member, such as a specific binding member that comprises or can bind a label such as a fluorophore. In this way generic reagents in the form of primary specific binding members (such as those that are coupled to or can directly or indirectly bind fluorophores or quenchers) that can specifically bind an attached tag can be used in the assays of the present invention. An important advantage of using attached tags is that it avoids the use of a specific binding member that binds an endogenous region of the target protein. Use of an endogenous region is not preferred, since an endogenous region could be a test compound binding site, or could be involved in heat-dependent aggregation of the target protein, or could be a region whose conformation or accessibility changes with sample heating. Examples of attached tags are short peptide tag sequences, such as, for example, the FLAG, hemagglutinin, myc, or 6xHis tags. Such tags can be inserted into a target protein sequence using recombinant DNA technology. Preferably, a peptide tag is added to a region of the protein such that it does not disrupt the native structure of a target protein and does not significantly alter the stability of the native structure of a target protein. For example, a peptide sequence tag can be added to the N or C terminus of a target protein. Optionally, short chemical or peptide linkers can be used to attach a peptide tag sequence to a target protein. Alternatively, an engineered epitope tag can be a chemical tag such as, for example, biotin or dinitrophenyl (DNP) that can be chemically attached to the N or C terminus of a protein. Thermal denaturation (assessed by CD or other methods) can be performed with target proteins having tags and the results compared with those of target proteins without tags to determine whether a tag sequence significantly affects the stability of a target protein.

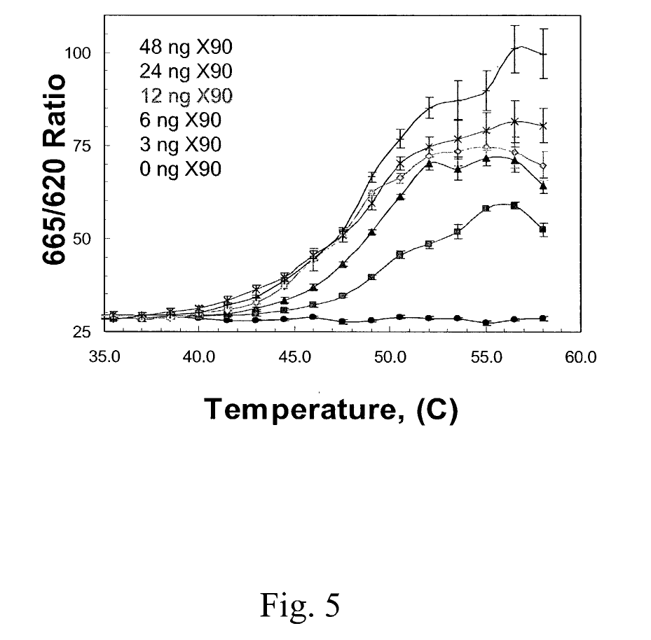

Preferably, a solution of a target molecule is made up, for example in a buffer, and the target molecule solution is added to one or more wells or sample containers. The amount of target molecule used in each sample will vary depending on the target. However, the high sensitivity/low background of the assay using FRET detection allows for very small amounts of target molecule to be used in these assays, for example, where the target molecule is a protein, from about 0.1 ng to 10 micrograms, but preferably the amount of target protein in an assay will be in the range of from about 1 ng to 100 ng. Typically, the concentration of protein in the assay will be in the sub-micromolar to micromolar range, such as from about 0.001 micromolar to about 100 micromolar, preferably from about 0.01 micromolar to about 50 micromolar. The optimal amount of a target protein in an assay sample can be determined empirically by titrating the amount of protein in the assay (see, for example, Example 2 and FIG. 5).

One or more test compounds is added to one or more wells or sample containers. Test compounds can be made up in solutions comprising buffers, solvents, or other compounds. Test compounds can be added to one or more wells before, after, or at the same time as target molecules are added to wells. Preferably, test compounds are added to a plurality of wells. It is within the scope of the invention to test several concentrations of the test compound in a given assay. It is also within the scope of the present invention to include more than one test compound in a single test well.

More than one test compound can be added to one or more wells. Preferably, test compounds added to at least two wells are different test compounds, or different amounts or combinations of test compounds. The amount of test compounds introduced into a well can vary, but in many cases will be in the sub-micromolar to micromolar range, such as from about 0.01 micromolar to about 100 micromolar, preferably from about 0.1 micromolar to about 50 micromolar.

Optionally, the assay mixtures of target molecule and test compounds are incubated for a period of time prior to the denaturation step. The incubation can be done at any temperature, but, if performed, the pre-incubation is preferably performed at a temperature of not more than 37 degrees C., and more preferably is performed at about 22 degrees C. The pre-denaturation incubation can be for any length of time, but in cases where it is included, it will typically be for 30 minutes or less.

Preferably, at least one control well comprising the target molecule in the absence of a test compound is included in the assay. Preferably the assay is performed on at least one control well at the same time as the test wells, and all steps of the assay are performed exactly as for the test well or wells; however, it is within the scope of the invention to perform control assays separately, and to record the control data for comparison with test compound assay measurements. One or more measurements from control wells, and values based on measurements from control wells (for example, averages, ratios, anisotropy etc.) whether assayed at the same time as the test wells or not, can be used as a reference value for comparison with one or more test wells.

In the alternative or in addition to including a control well, it is possible to include at least one standard well that comprises a target molecule and at least one compound. The interaction of the compound in the standard well with the target molecule may not be known in advance of the assay, but preferably the degree to which the standard well compound affects denaturation of the target protein is known. In some aspects, standard wells can be test compound wells that are compared with other test compound wells in the assays of the present invention. Preferably, where one or more standard wells is used, the assay is performed on at least one standard well at the same time as the test wells, and all steps of the assay are performed exactly as for the test well or wells; however, it is within the scope of the invention to perform standard assays separately, and to record the standard well data for comparison with test compound assay measurements. One or more measurements from standard wells, and values based on measurements from standard wells (for example, averages, ratios, anisotropy etc.) whether assayed at the same time as the test wells or not, can be used as a reference value for comparison with one or more test wells.

A first specific binding member that comprises or can directly or indirectly bind a member of a FRET pair (i.e., a FRET donor or a FRET acceptor) can be added to the target molecule solution before or after the target protein solution is added to the well. The first specific binding member can also be added after the samples have been subjected to denaturing conditions. This first specific binding member specifically binds the unfolded form of the target molecule, for example, by binding an epitope of the target molecule that is exposed or formed when the target molecule unfolds. For protein targets, the first specific binding member is preferably an antibody, such as a monoclonal antibody. Antibody fragments that retain the specific binding activity of a monoclonal antibody can also be used (for example Fab fragments).

The first specific binding member that specifically binds the unfolded form of the target molecule comprises or can directly or indirectly bind a member of a FRET pair. For example, a specific binding member can be conjugated to a fluorophore or quencher using methods known in the art. Alternatively, the specific binding member can indirectly bind a fluorophore or quencher, for example, through the use of one or more other specific binding member pairs (hereinafter called “secondary specific binding members”, where “primary specific binding members” are those that directly bind the target molecule). One example of a secondary specific binding member pair that can be used to link a fluorophore or quencher to a primary specific binding member such as an antibody used in the ATLAS assay is biotin-streptavidin. For example, a fluorophore can be linked to streptavidin, and a primary specific binding member used in the assay can be biotinylated (or vice versa). This mechanism of linking a fluorophore (or quencher) to a primary specific binding member such as an antibody can provide flexibility in the assay, such that the fluorophore (or quencher) can optionally be added to the assay mixture at a different time from the addition of the first specific binding member is added (for example, after heating to T_{ATLAS} and subsequent cooling to room temperature, and before signal detection). Other secondary specific binding member pairs that can be used include biotin-avidin, chitin binding domain-chitin binding protein; nitroloacetic acid-6xHis; calmodulin binding domain-calmodulin; etc. It is also possible to use antibodies as secondary specific binding members, for example, isotype- and species-specific secondary antibodies can bind be conjugated to a fluorophore or quencher and can bind primary antibodies used to bind the target protein. A specific binding member that “can directly or indirectly bind” a FRET donor or a FRET acceptor can be bound to a FRET donor or a FRET acceptor when the specific binding member is added to the test well, or can be not bound to a FRET donor or a FRET acceptor when the specific binding member is added to the test well. If the specific binding member is not bound to a FRET pair member when it is added to the test well, it can bind a FRET pair member upon contacting the specific binding member with the FRET pair member (such as in the test well). The binding can optionally be mediated by secondary specific binding members.

The one or more wells are subjected to conditions at which at least a portion of the target protein is unfolded in the absence of a ligand or test compound. Denaturing conditions can be any conditions that cause loss of secondary, tertiary, or quaternary structure of a target molecule, or alter the three-dimensional conformation of a target molecule, including heat, pH changes, presence of detergents or surfactants, chaotropic agents, salts, chelators, etc. Preferably, the denaturing conditions are elevated temperature and subjecting the test wells to denaturing conditions comprises heating the target molecule and one or more test compounds to one or more predetermined temperatures at which at least a portion of said target molecule is denatured.

In preferred aspects of the present example, the test wells and any control or standard wells will be heated to a single discrete predetermined temperature, termed T_{ATLAS}. T_{ATLAS} can be selected in preliminary experiments in which the target molecule is heated and its degree of unfolding as a function of temperature is monitored (although the identity or any activity of the target molecule need not be known). Preferably, before the assay is performed, the target molecule is characterized to establish a melting (temperature dependent structural unfolding) curve in which a physical measurement that reports on the target molecule's structure is plotted as a function of temperature. The physical measurement can be based on any of a variety of structural determination methods well known in the art, for example, CD, light scattering, UV absorption spectroscopy, differential scanning calorimetry, etc. The melting curve of a target molecule can then used to establish the parameters, including T_{ATLAS} of the assay. Thermal melting can preferably be performed under assay conditions (using buffers, reagents, specific binding members, donor fluorophores, acceptor moieties, and FRET detection that will be used in test compound assays) to obtain a melting curve under assay conditions (in the absence of test compounds) (see Example 2 and FIG. 5). Preferably, T_{ATLAS} will be selected as a temperature at which assay reagents are stable and the assay has a wide dynamic range and high quality (Z′).

In some cases, it may be desirable to heat the wells to more than one discrete temperature (e.g., T_{ATLAS1}, T_{ATLAS2}, etc.), but this is less preferred. This can be desirable in some cases, for example, if melting curves demonstrate that the target molecule has more than one transition temperature that is indicative of unfolding intermediates. Preferably, however, no more than three discrete temperatures are used in the ATLAS assay, and most preferably the wells are heated to a single T_{ATLAS}.

Heating can be performed in any incubator or sample heating device and is preferably performed using a heating device that allows for rapid, uniform, and accurate heating, and preferably cooling, to precise temperatures, as well as accurate temperature maintenance. For example, many commercially available thermocyclers can be used for this purpose. The assay samples can be held at T_{ATLAS} for any period of time, for example from about 3 minutes to about 6 hours, preferably from about 10 minutes to about one hour. However, the time of T_{ATLAS} incubation is not a limitation of the present invention.

The samples are optionally cooled to a temperature less than T_{ATLAS}. In most cases, assay samples are cooled to approximately room temperature (22 degrees C.). Preferably, where cooling is employed, it is relatively rapid and occurs at a defined rate. In the alternative, it is also possible to maintain the samples at T_{ATLAS} for the detection step. This requires that the fluorescence detection means can interface with a heating element that can maintain the desired temperature during fluorescence detection.

Before or after heating to T_{ATLAS}, and, optionally but preferably, cooling the samples to a lower temperature, a second specific binding member is added to one or more test wells, and, preferably, to a control (or standard) well or wells. The second specific binding member can comprise or bind a FRET donor or a FRET acceptor. The second specific binding member specifically binds the target molecule at a site distinct from the binding site recognized by the first specific binding member. In cases where target molecules are proteins, the binding site of the second specific binding member is preferably an attached tag, such as an attached peptide tag, for example, the 6xHis, myc, FLAG, or hemaglutinin tag, or any other short peptide sequence known in the art or later developed that can be specifically recognized by a specific binding member. The use of engineered peptide sequence tags introduced into target proteins allows the use of generic antibody reagents in ATLAS assays, where a generic antibody reagent can be an antibody that recognizes the attached tag and is directly or indirectly coupled to a fluorophore or quencher. A generic antibody reagent can be used in ATLAS for any target protein that comprises the attached tag recognized by the antibody. The use of an attached tag also avoids the possibility that the second specific binding member competes with a test compound for binding a particular region of the target molecule or binds an endogenous region of the target protein that is altered during denaturation.

In assays in which the first specific binding member comprises or binds a FRET donor, the second specific binding member preferably binds or comprises a FRET acceptor. In assays in which the first specific binding member comprises or binds a FRET acceptor, the second specific binding member preferably binds or comprises a FRET donor. As in the case of the first specific binding member, the second specific binding member used in the assay can be directly or indirectly coupled to the fluorophore or quencher. Direct coupling can be, for example, chemical coupling of the fluorophore through active groups on the specific binding member. Indirect coupling can use further secondary specific binding members, such as biotin and streptavidin, that can bind the second specific binding member and the fluorophore, such that the second specific binding member and the fluorophore can be coupled together through biotin-streptavidin binding.

In some preferred aspects of this embodiment of the present invention, a first specific binding member is present in the assay and control (and/or standard) samples during the heating, but is not bound to a fluorophore or quencher until after the assay and control samples have been heated to T_{ATLAS} and subsequently cooled to below 37 degrees C. The first specific binding member is coupled to a secondary specific binding member for linkage to a FRET partner. Prior to detection, a “Revelation Mix” is added to the assays that comprises a FRET partner that can bind the first specific binding member (through a secondary specific binding member) as well as the second specific binding member that is coupled to the other member of the FRET pair. The addition of fluorophores (and, optionally, quenchers) after the samples have been brought to a temperature below T_{ATLAS} and before fluorescence detection can avoid the possibility of interference of a fluorophore with unfolding of the target molecule, and can avoid potential problems due to heat-instability of fluorophores.

Taken together, the fluorophore that directly or indirectly binds or is integral to the first specific binding member and the fluorophore that directly or indirectly binds or is integral to the second specific binding member form a FRET pair. Nonlimiting examples of FRET pairs that can be useful in the methods of the present invention include terbium/fluorescein, terbium/GFP, terbium/TMR, terbium/Cy3, terbium/R phycoerythrin, Europium/Cy5, Europium/APC, Alexa 488/Alexa 555, Alexa 568/Alexa 647, Alexa 594/Alexa 647, Alexa 647/Alexa 594, Cy3/Cy5, BODIPY FL/BODIPY FL, Fluorescein/TMR, IEDANS/fluorescein, fluorescein/fluorescein, and EDANS/DABCYL. Other FRET pairs comprising a fluorescence donor and an acceptor moiety that are known or become known in the art can also be used. In selecting FRET pairs, donors and acceptors should be chosen in which the donor emission wavelength spectrum overlaps the acceptor absorption wavelength spectrum. In addition, for optimal assay sensitivity, the distance the donor and acceptor will be positioned from each other when both are bound to the target molecule according to the methods of the present invention is preferably less than or equal to the Forster radius of the pair. FRET pairs can be selected based on these criteria (fluorescence spectra and Forster radius values) can be found in the literature (Principles of Fluorescence Spectroscopy, 2^{nd} edition (1999) ed. by Joseph R. Lakowicz, Plenum Publishing Corp.; and literature available from Molecular Probes, Eugene, Oreg. and available at www.probes.com) and tested for their appropriateness and efficacy in assays configured with the test protein thermally melted in the absence of test compound.

The ATLAS assay further includes detecting fluorescence emission at one or more wavelengths from one or more wells comprising target molecule and test compound and at least one control wells or one or more standard wells. The fluorescence emission detected in the assay is the result of the interaction between two FRET partners, either a fluorescence donor and a fluorescence acceptor, or a fluorescence donor and a quencher. The assay is configured such that denaturation of a target molecule allows binding of a first member of the FRET pair, and binding of the FRET partner to a site of the target molecule distinct from the site bound by the first member of the FRET pair brings the FRET partners into proximity. Thus the extent of thermal denaturation of the target molecule determines the intensity or wavelength properties of the fluorescence signal.

The detection of the fluorescence signal can be at one or more wavelengths. For example, the detection of fluorescence can be at the wavelength of the donor fluorophore, where reduced intensity of the fluorescence of the donor fluorophore depends on its proximity to an acceptor fluorophore or quencher. More preferably, the detection of fluorescence can be at the wavelength of an acceptor fluorophore.

Preferably, the detection is fluorescence resonance energy transfer (FRET) detection, where the assay is designed to detect fluorescence of an acceptor fluorophore. More preferably the assay detects fluorescence of both the donor and the acceptor fluorophore of an acceptor/donor pair. Fluorescence of the donor and acceptor can be expressed as a ratio, for example the ratio of fluorescence at the acceptor emission wavelength to fluorescence at the donor emission wavelength. It is also possible, however, to assay protein unfolding by detecting fluorescence emission at the donor wavelength. For example, fluorescence at the donor wavelength will be reduced by increased protein unfolding as the fluorescence donor can be brought into proximity with a fluorescence acceptor or fluorescence quencher.

Fluorescence detection can be performed by any device that can detect fluorescence at the wavelength emitted by the fluorophore used in the assay. Fluorescence detection devices, including those that detect fluorescence from multiwell plates, are known in the art (for example the Victor V manufactured by Perkin Elmer and the Fusion analyzer manufactured by Packard Biosciences). The fluorescence detection device can interface with the sample heating device, or can be separate. Preferably, the fluorescence detection device can detect fluorescence at more than one wavelength, and preferably includes software that can calculate a ratio between two wavelengths, such as the wavelengths of fluorescence emission of a donor and acceptor used in the assay.

Detection of fluorescence emission at one or more wavelengths is preferably time-resolved fluorescence detection. A preferred detection mechanism used in the methods of the present invention uses time-resolved fluorescence detection at two wavelengths, and thus can be referred to as “time resolved energy transfer” or “TRET”, or “time-resolved fluorescence resonance energy transfer” or “TR-FRET”. TRET (or “TR-FRET”) detection is well known in the art (Pope et al. (1999) Drug Disc Tech 4 (8): 350-362). As practiced in the present invention, TR-FRET involves delaying the measurement of fluorescence intensity at two or more wavelengths by a short time window after excitation of the donor fluorophore. This can reduce the background due to compound interference in fluorescence measurements.

In preferred aspects of the present invention, one or more control wells is made up that lacks a test compound, but that comprises the target molecule and specific binding member(s) in the same amounts as the test wells, and the control well is heated and analyzed in the same way and at the same time as the test wells. Preferably, one or more control wells is in a multiwell plate that also contains test wells, and the test compound and control assay mixtures are made up at the same time from the same stock concentrations of target molecule, specific binding members, signal molecules, etc.

In the alternative, one or more control wells can be made up at a time other than that when test wells are made up. One or more control wells can be heated and subjected to fluorescence detection measurements, before or after the test wells are heated. The data from the fluorescence detection of a control well can be recorded and stored, such as in a database.

In some aspects of the present invention, one or more standard wells are provided for comparison with one or more test wells. Standard wells comprise target protein and at least one compound that is either a test compound or a compound whose affect on target unfolding is known. One or more standard wells is also heated and analyzed in the same way and preferably at the same time as the test wells. Where standard wells are used to generate a reference value, they can be one, some, or all of the test wells in one or more assays, and can be used to compute an average value of a detection measurement against which individual test well detection measurements can be compared. Preferably, in aspects of the invention in which standard wells are used, at least one standard well is in a multiwell plate that also contains test wells, and the test compound and standard assay mixtures are made up at the same time from the same stock concentrations of target molecule, specific binding members, signal molecules, etc.

In the alternative, standard wells can be made up at a time other than that when test wells are made up. One or more standard wells can be heated and subjected to fluorescence detection measurements, before or after the test wells are heated. The data from the fluorescence detection of a standard well can be recorded and stored, such as in a database.

Determination of Target Molecule Unfolding

Measurements from test wells are compared with measurements from one or more control or one or more standard wells to determine whether any test compounds significantly alter the fluorescence readout. For example, test wells that differ from control wells by more than a particular amount or percentage in fluorescence intensity at one or more wavelengths, or by more than a particular amount or percentage in a ratio of fluorescence intensity at two or more wavelengths, can be identified as wells in which the target molecules has unfolded to a significantly different degree than in control wells lacking test compound. Test wells that differ from standard wells by more than a particular amount or percentage in fluorescence intensity at one or more wavelengths, or by more than a particular amount or percentage in a ratio of fluorescence intensity at two or more wavelengths, can be identified as wells in which the target molecules has unfolded to a significantly different degree than in standard wells comprising one or more different compounds. The comparison between test compound and control wells or standard wells can be a comparison of fluorescence intensity (or a value derived therefrom) at a fluorescence donor emission wavelength, a comparison of fluorescence intensity (or a value derived therefrom) at a fluorescence acceptor emission wavelength, or a comparison of some value that is a function of both fluorescence donor emission wavelength and fluorescence acceptor emission wavelength. Preferably, where the assay uses a FRET pair comprising a fluorescence donor and a fluorescence acceptor, the comparison is based on a ratio of fluorescence acceptor emission to fluorescence donor emission. Preferably, where the assay uses a FRET pair comprising a fluorescence donor and a fluorescence quencher, the comparison is based on donor wavelength emission intensities.

In most (but not all) cases, a significant difference in fluorescence signal or signals or determinations based on fluorescence signals will indicate that a test compound has to some degree protected the target molecule from unfolding in response to denaturing conditions such as elevated temperature. In the case of a fluorescence donor/fluorescence acceptor pair, a reduction in the ratio of acceptor to donor fluorescence is indicative of a reduction in target unfolding in the presence of test compound. In the case of a fluorescence donor/fluorescence quencher pair, an increase in the intensity of donor fluorescence is indicative of a reduction in target unfolding in the presence of test compound. It is also possible to identify compounds that promote unfolding of the target by detecting an increase in the ratio of acceptor to donor fluorescence or, in the case of a donor/quencher pair, a decrease in the intensity of donor fluorescence. Compounds that promote unfolding of the target can also be ligands of the target. Without being bound to a particular mechanism, in some cases compound binding may make a target more susceptible to unfolding at a particular temperature.

Identification of Ligands

Test compound wells that differ from control wells or standard wells by more than a particular amount or percentage in fluorescence intensity at one or more wavelengths, or by more than a particular amount or percentage in a ratio of fluorescence intensity at two or more wavelengths, can be identified as wells that comprise test compounds that protect the target molecule from unfolding at elevated temperature. Test compounds identified as stabilizing the target molecule at high temperature are identified as potential ligands of the target molecule. Those skilled in the art can determine reasonable criteria for identifying first screen ligands, such as, for example 20% or greater difference from control data, or preferably a 50% or greater difference from control data.

Preferably, first screen hits are rescreened in the same assay format in which they were originally identified. First screen hits that differ from control wells or standard wells by more than a particular amount or percentage in fluorescence intensity at one or more wavelengths, or by more than a particular amount or percentage in a ratio of fluorescence intensity at two or more wavelengths, in a second assay are called duplicate hits.

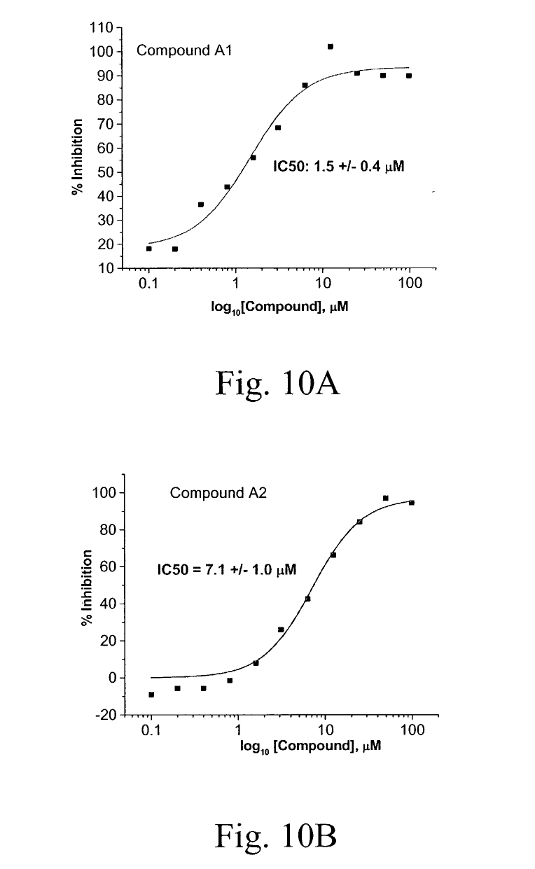

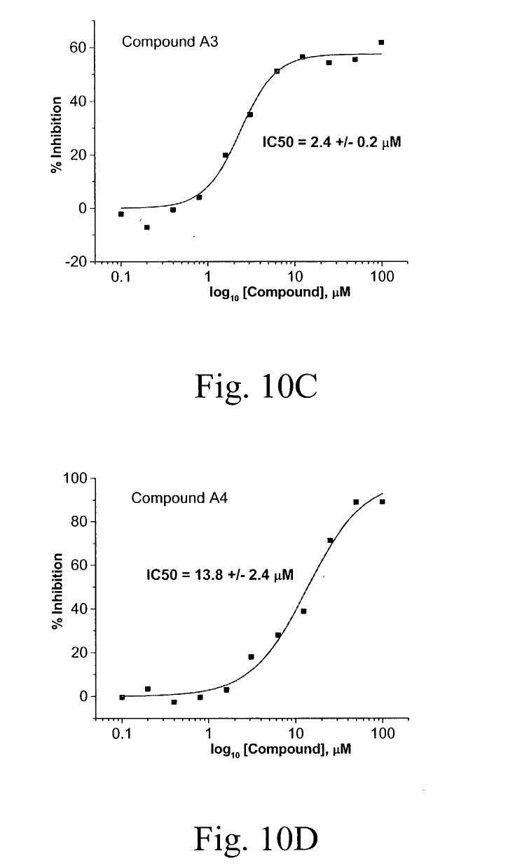

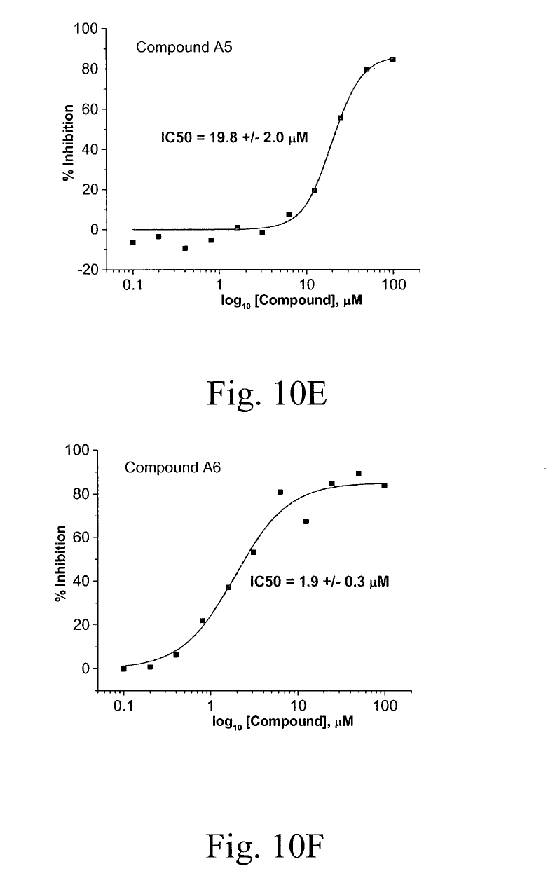

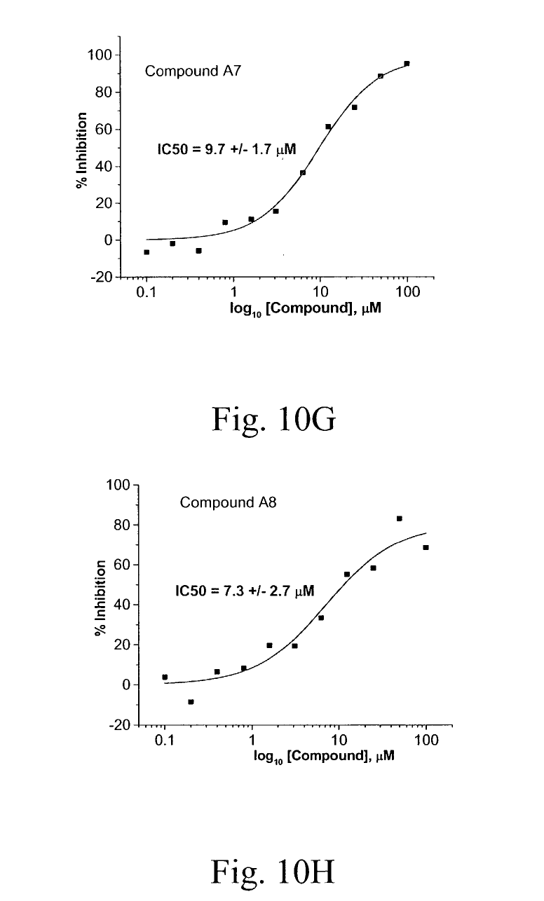

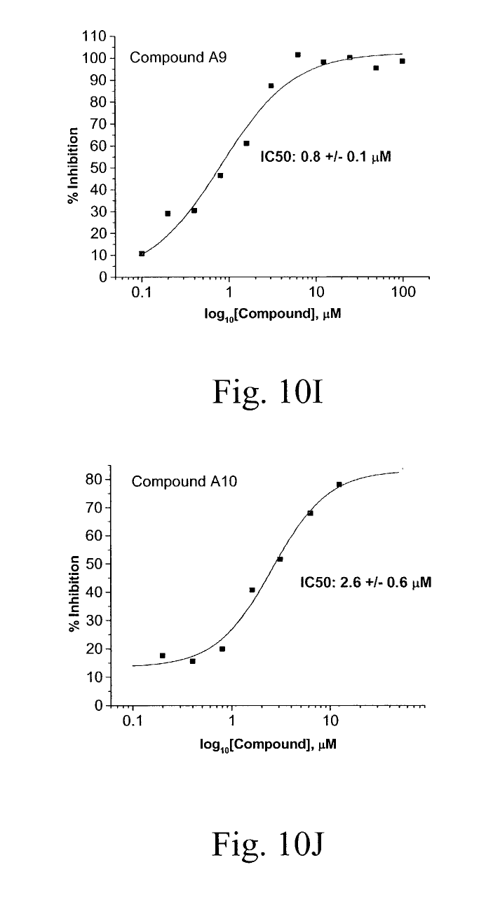

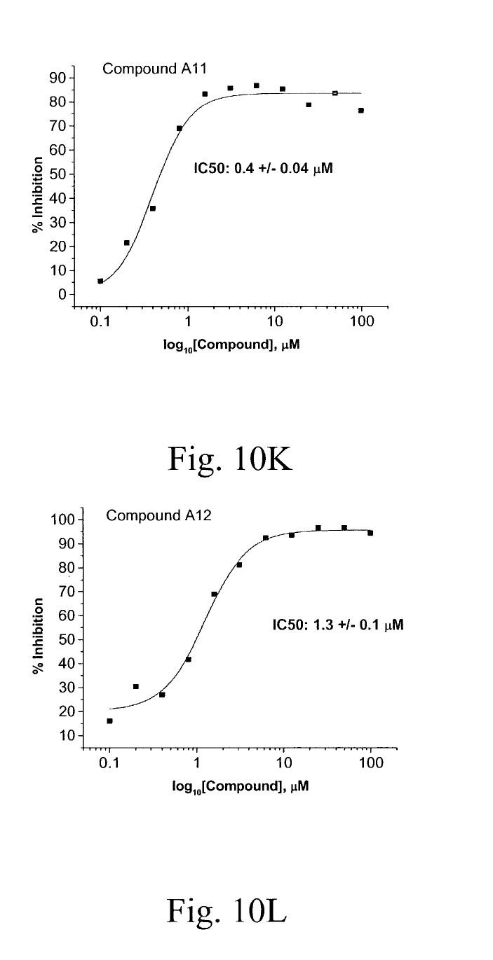

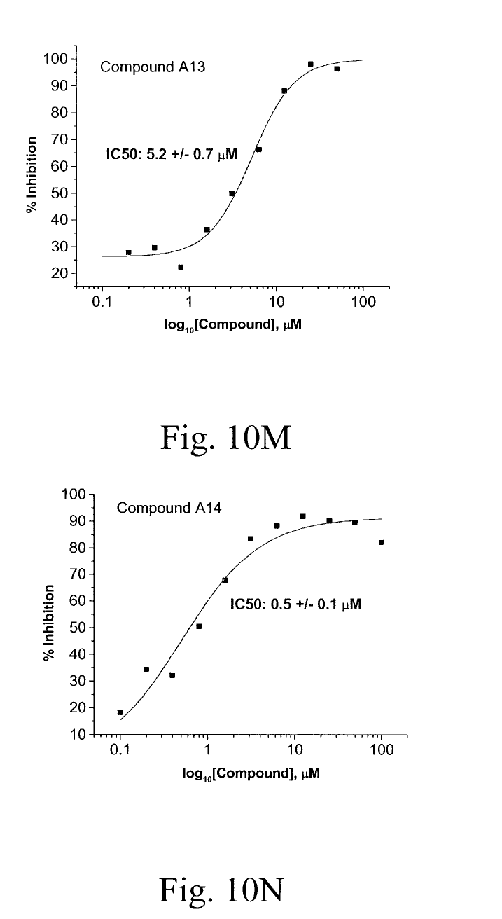

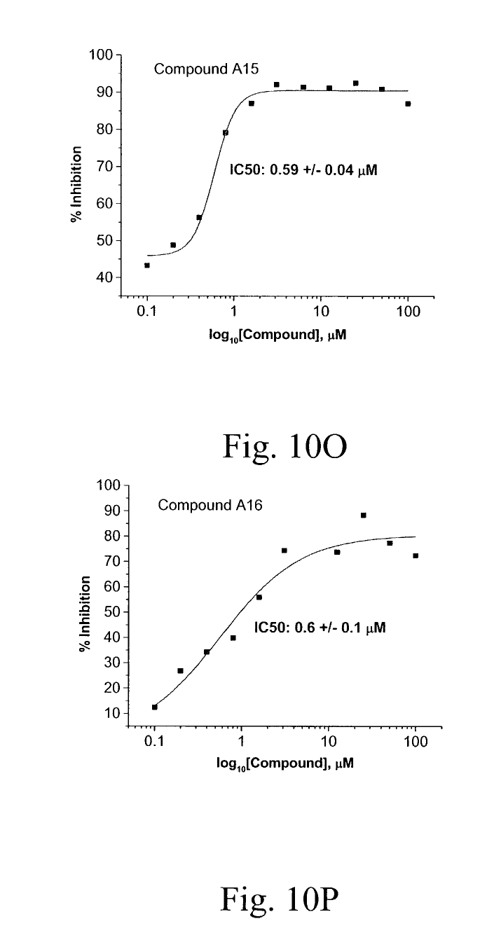

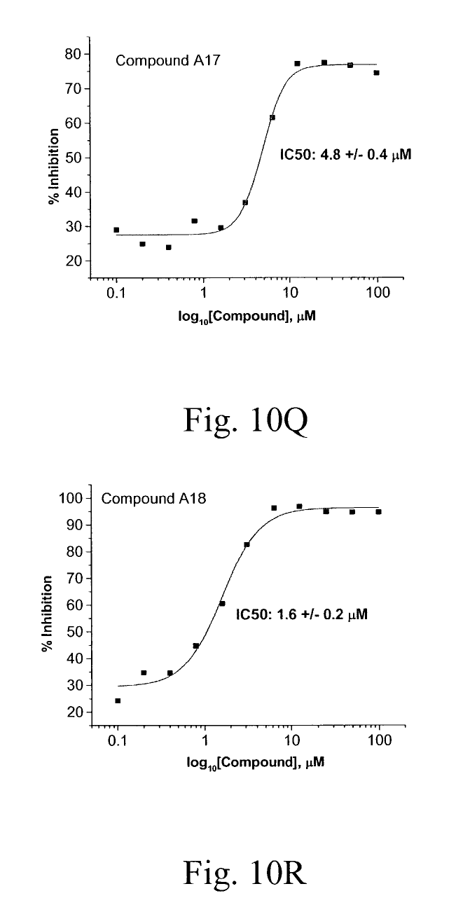

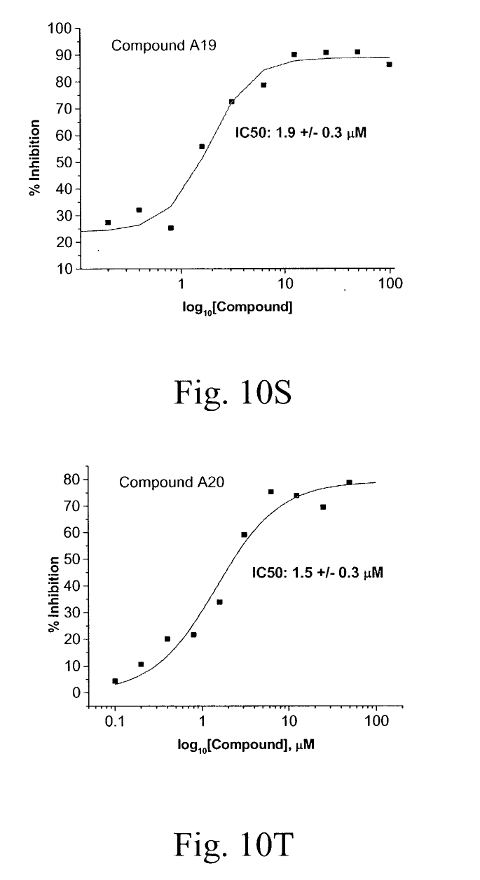

Duplicate hits can be subjected to a titration series in which they assayed at a range of concentrations (see Example 5). Duplicate hits that are titratable, that is, that show concentration dependency in the assay, are identified as putative ligands for the target. IC 50 values can be determined from these assays (see, for example, FIG. 10).

Test compounds identified as target molecule ligands can be tested in other types of assays for independent confirmation of target molecule binding. Examples of such assays are ELISA, gel filtration, filter binding, isothermal calorimetry, and other binding assays as they are known in the art.

High Throughput Screening

The present invention is particularly well-suited to high throughput screening, in which a multiplicity of test compounds can be tested at the same time. Because of the high degree of sensitivity and low background of FRET detection, and particulary TR-FRET detection, small amounts of protein and correspondingly small volumes (for example, less than 20 microliters) can be used for assays. In high throughput assays, samples are preferably made up in wells of multiwell plates. However, other sample containers can be used. For example, the sample containers can be indentations of a surface, or can be capillaries or tubes for holding small volume (sub-milliliter) liquid samples. Preferably, the assay is formatted for high throughput or ultra high throughput screening (HTS or UHTS) involving a multiplicity, and preferably hundreds, of samples, and thus the assays are most conveniently performed in wells of for example, 96, 384, 1536, or 3456 well plates. Plate heating and plate fluorescence detection systems as they are known in the art or designed for the methods of the present invention can be used.

The ATLAS assay can easily be configured such that a minimum of pipeting steps are required. For example, in Example 4, three reagent mixes are used: one containing test compound, one containing target protein and the first specific binding member, and one containing the “revelation mix” of fluorophores, secondary specific binding members, and a second specific binding member. Preferably, liquid handling devices are used for dispensing sample components. In addition, the assay can be performed within a short time period, as assay samples can be assembled, rapidly heated to a single temperature, incubated for less than an hour, rapidly cooled, and detected.

The addition of reagents, as well as heating, incubations, cooling and detection steps can be automated. In a preferred aspect of the present invention, an integrated system employs robotics to dispense reagents, and to move plates comprising test wells to and from dispensing areas, heating/cooling devices, and fluorescence plate readers. Preferably the integrated system is computerized and programmable, and contains software for sample analysis.

II. Methods of Screening to Identify One or more Compounds that Bind to a Target Molecule Using a Specific Binding Member that Recognizes the Unfolded Form of a Target Molecule and FP Detection

Another embodiment of the present invention is a screening method for identifying one or more ligands of a target molecule in which the screening method uses a specific binding member that specifically recognizes the unfolded form of a target molecule and fluorescence polarization (PP) detection. By “specifically recognizes” is meant that the specific binding member binds target molecules that are in the unfolded state, but does not appreciably bind target molecules that are in the folded, or native, state. The target molecule is contacted with at least one test compound and subjected to a denaturing treatment in the presence of a specific binding member specific for the unfolded form of the target molecule (hereinafter referred to as an “unfolded-specific binding member”). As the protein unfolds, the unfolded-specific binding member binds to unfolded target molecules. After the denaturation step, fluorescence polarization is detected, and when compared with reference values, the fluorescence measurement is used as an indicator of the degree to which the target molecule occurs in the unfolded state at the assay temperature. Test compounds that alter the degree to which the target molecule occurs in the unfolded state at the assay temperature are identified as ligands of a target protein.

The method includes: providing a target molecule in solution in one or more test wells; adding to the one or more test wells one or more test compounds; adding to the one or more test wells a specific binding member that specifically binds the unfolded form of the target molecule, where the first specific binding member comprises or can directly or indirectly bind a fluorophore; and subjecting the one or more test wells to conditions at which at least a portion of the target molecule is denatured. The method further includes measuring fluorescence polarization from the one or more test wells; making a comparison of fluorescence polarization of one or more test wells with a reference value; using said comparison of fluorescence polarization to determine the extent to which the target molecule occurs in the unfolded state, the folded state, or both, in the wells comprising target molecules and test compounds; and using the determination of the extent to which said target molecule occurs in the unfolded state, the folded state, or both, in the wells comprising target molecule and test compounds to determine whether one or more test compounds binds said target molecule, thereby identifying one or more ligands of the target molecule.

The target molecule for which ligands are sought can be any molecule, but preferably the target molecule is a biomolecule, more preferably a biomolecule that comprises a peptide or a protein, and most preferably a biomolecule that comprises a protein. A biomolecule that comprises a protein or peptide can be, for example, a protein that comprises chemical or post-translational modifications, and can be for example, a glycoprotein, nucleoprotein, lipoprotein, farnsylated, meristylated, acylated, phosphorylated, or sulfated protein, etc. Where “protein” or “target protein” is used herein, the aforementioned biomolecules that comprise protein are also included. It can also include peptide or chemical moieties such as but not limited to linkers, labels (including fluorophores), tags, and specific binding members.

Target proteins can be of any species origin and can be isolated from native sources, including organisms, environmental sources, or media, or can be produced using recombinant technologies using endogenous or exogenous cell types. For example, target proteins can be produced in bacterial or fungal cultures, insect cell cultures, avian cell cultures, mammalian (including human) cell cultures, etc. They can also be produced by transgenic organisms. The proteins are preferably at least partially purified, and more preferably substantially purified, for use in assays. The proteins can differ in sequence with regard to the native wild-type form, and can include one or more attached tags.

In preferred aspects of the present invention, a target protein can include an attached tag that can be recognized by a specific binding member, such as a specific binding member that comprises or can bind a label such as a fluorophore. In this way generic reagents in the form of primary specific binding members that can specifically bind an attached tag can be used in the assays of the present invention. An important advantage of using attached tags is that it avoids the use of a specific binding member that binds an endogenous region of the target protein. Use of an endogenous region is not preferred, since an endogenous region could be a test compound binding site, or could be involved in heat-dependent aggregation of the target protein, or could be a region whose conformation or accessibility changes with sample heating. Examples of attached tags are short peptide tag sequences, such as, for example, the FLAG, hemagglutinin, myc, or 6xHis tags. Such tags can be inserted into a target protein sequence using recombinant DNA technology. Preferably, a peptide tag is added to a region of the protein such that it does not disrupt the native structure of a target protein and does not significantly alter the stability of the native structure of a target protein. For example, a peptide sequence tag can be added to the N or C terminus of a target protein. Optionally, short chemical or peptide linkers can be used to attach a peptide tag sequence to a target protein. Alternatively, an engineered epitope tag can be a chemical tag such as, for example, biotin or dinitrophenyl (DNP) that can be chemically attached to the N or C terminus of a protein. Thermal denaturation (assessed by CD or other methods) can be performed with target proteins having tags and the results compared with those of target proteins without tags to determine whether a tag sequence significantly affects the stability of a target protein.

Preferably, a solution of a target molecule is made up, for example in a buffer, and the target molecule solution is added to one or more wells or sample containers. The amount of target molecule used in each sample will vary depending on the target. However, the high sensitivity/low background of the assay using FP detection allows for very small amounts of target molecule to be used in these assays, for example, where the target molecule is a protein, from about 0.1 ng to 10 micrograms, but preferably the amount of target protein in an assay will be in the range of from about 1 ng to 5 micrograms. The optimal amount of a target protein in an assay sample can be determined empirically by titrating the amount of protein in the assay.

One or more test compounds is added to one or more wells or sample containers. Test compounds can be made up in solutions comprising buffers, solvents, or other compounds. Test compounds can be added to one or more wells before, after, or at the same time as target molecules are added to wells. Preferably, test compounds are added to at least two wells. It is within the scope of the invention to test several concentrations of the test compound in a given assay. It is also within the scope of the present invention to include more than one test compound in a single test well.

More than one test compound can be added to one or more wells. Preferably, test compounds added to at least two wells are different test compounds, or different amounts or combinations of test compounds. The amount of test compounds introduced into a well can vary, but in many cases will be in the sub-micromolar to micromolar range, such as from about 0.01 micromolar to about 500 micromolar.

Optionally, the assay mixtures of target molecule and test compounds are incubated for a period of time prior to the denaturation step. The incubation can be done at any temperature, but, if performed, the pre-incubation is preferably performed at a temperature of not more than 37 degrees C., and more preferably is performed at about 22 degrees C. The pre-denaturation incubation can be for any length of time, but in cases where it is included, it will typically be for 30 minutes or less.

Preferably, at least one control well comprising the target molecule in the absence of a test compound is also included in the assay. Preferably the assay is performed on at least one control well at the same time as the test wells, and all steps of the assay are performed exactly as for the test well or wells; however, it is within the scope of the invention to perform control assays separately, and to record the control data for comparison with test compound assay measurements. One or more measurements from control wells, and values based on measurements from control wells (for example, averages, ratios, anisotropy etc.) whether assayed at the same time as the test wells or not, can be used as a reference value for comparison with one or more test wells.

In the alternative or in addition to including a control well, it is also possible to include at least one standard well that comprises a target molecule and at least one compound. The interaction of the compound in the standard well with the target molecule may not be known in advance of the assay, but preferably the degree to which the standard well compound affects denaturation of the target protein is known. In some aspects, standard wells can be test compound wells that are compared with other test compound wells in the assays of the present invention. Preferably, where one or more standard wells is used, the assay is performed on at least one standard well at the same time as the test wells, and all steps of the assay are performed exactly as for the test well or wells; however, it is within the scope of the invention to perform standard assays separately, and to record the standard well data for comparison with test compound assay measurements. One or more measurements from standard wells, and values based on measurements from standard wells (for example, averages, ratios, anisotropy etc.) whether assayed at the same time as the test wells or not, can be used as a reference value for comparison with one or more test wells.

An unfolded-specific binding member that comprises or can directly or indirectly bind a fluorophore can be added to the target molecule solution before or after the target protein solution is added to the well. The first specific binding member can also be added after the samples have been subjected to denaturing conditions. This first specific binding member specifically binds the unfolded form of the target molecule, for example, by binding an epitope of the target molecule that is exposed or formed when the target molecule unfolds. The unfolded-specific binding member is preferably an antibody, such as a monoclonal antibody. Antibody fragments that retain the specific binding activity of a monoclonal antibody can also be used (for example Fab fragments).

The unfolded-specific binding member that binds the unfolded form of the target molecule comprises or can directly or indirectly bind a fluorophore. For example, a specific binding member can be conjugated to a fluorophore using methods known in the art. Alternatively, the specific binding member can indirectly bind a fluorophore, for example, through the use of one or more other specific binding member pairs (hereinafter called, “secondary specific binding members”, where “primary specific binding members” are those that directly bind the target molecule). One example of a secondary specific binding member pair that can be used to link a fluorophore or quencher to a primary specific binding member such as an antibody used in the assays of the present invention is biotin-streptavidin. For example, a fluorophore can be linked to streptavidin, and a primary specific binding member used in the assay can be biotinylated (or vice versa). This mechanism of linking a fluorophore to a primary specific binding member such as an antibody can provide flexibility in the assay, such that the fluorophore can optionally be added to the assay mixture at a different time from the addition of the first specific binding member is added (for example, after heating to T_{ATLAS} and subsequent cooling to room temperature, and before signal detection). Other secondary specific binding member pairs that can be used include biotin-avidin, chitin binding domain-chitin binding protein; nitroloacetic acid-6xHis; calmodulin binding domain-calmodulin; etc. It is also possible to use antibodies as secondary specific binding members, for example, isotype- and species-specific secondary antibodies can bind be conjugated to a fluorophore or quencher and can bind primary antibodies used to bind the target protein. An unfolded-specific binding member that can directly or indirectly bind a fluorophore can be bound to a fluorophore when the specific binding member is added to the test well, or can be not bound to a fluorophore when the unfolded-specific binding member is added to the test well. If the unfolded-specific binding member is not bound to a fluorophore when it is added to the test well, it can bind a fluorophore upon contact with the fluorophore (such as in the test well). The binding can optionally be mediated by secondary specific binding members.

In a variation of this method, the target molecule can be directly labeled with a fluorophore. In this aspect of the present embodiment, binding of an unfolded-specific binding member changes the size of the target molecule complex, and thus increases the FP signal of the target molecule.

The one or more wells are subjected to conditions at which at least a portion of the target protein is unfolded in the absence of a ligand or test compound. Denaturing conditions can be any conditions that cause loss of secondary, tertiary, or quaternary structure of a target molecule, or alter the three-dimensional conformation of a target molecule, including heat, pH changes, presence of detergents or surfactants, chaotropic agents, salts, chelators, etc. Preferably, the denaturing conditions are elevated temperature and subjecting the test wells to denaturing conditions comprises heating the target molecule and one or more test compounds to one or more predetermined temperatures at which at least a portion of said target molecule is denatured.

In preferred aspects of the present example, the test wells and any control or standard wells will be heated to a single discrete predetermined temperature, termed T_{ATLAS}. T_{ATLAS} can be selected in preliminary experiments in which the target molecule is heated and its degree of unfolding as a function of temperature is monitored (although the identity or any activity of the target molecule need not be known). Preferably, before the assay is performed, the target molecule is characterized to establish a melting (temperature dependent structural unfolding) curve in which a physical measurement that reports on the target molecule's structure is plotted as a function of temperature. The physical measurement can be based on any of a variety of structural determination methods well known in the art, for example, CD, light scattering, UV absorption spectroscopy, differential scanning calorimetry, etc. The melting curve of a target molecule can then used to establish the parameters, including T_{ATLAS} of the assay. Thermal melting can preferably be performed under assay conditions (using buffers, reagents, specific binding members, donor fluorophores, acceptor moieties, and FRET detection that will be used in test compound assays) to obtain a melting curve under assay conditions (in the absence of test compounds). Preferably, T_{ATLAS} will be selected as a temperature at which assay reagents are stable and the assay has a wide dynamic range and high quality (Z′).

In some cases, it may be desirable to heat the wells to more than one discrete temperature (e.g., T_{ATLAS1}, T_{ATLAS2}, etc.), but this is less preferred. This can be desirable in some cases, for example, if melting curves demonstrate that the target molecule has more than one transition temperature that is indicative of unfolding intermediates. Preferably, however, no more than three discrete temperatures are used in the ATLAS assay, and most preferably the wells are heated to a single T_{ATLAS}.

Heating can be performed in any incubator or sample heating device and is preferably performed using a heating device that allows for rapid, uniform, and accurate heating, and preferably cooling, to precise temperatures, as well as accurate temperature maintenance. For example, many commercially available thermocyclers can be used for this purpose. The assay samples can be held at T_{ATLAS} for any period of time, for example from about 3 minutes to about 6 hours, preferably from about 10 minutes to about one hour. However, the time of T_{ATLAS} incubation is not a limitation of the present invention.

The samples are optionally cooled to a temperature less than T_{ATLAS}. In most cases, assay samples are cooled to approximately room temperature (22 degrees C.). Preferably, where cooling is employed, it is relatively rapid and occurs at a defined rate. In the alternative, it is also possible to maintain the samples at T_{ATLAS} for the detection step. This requires that the fluorescence detection means can interface with a heating element that can maintain the desired temperature during fluorescence detection.

Before or after heating to T_{ATLAS}, and, optionally but preferably, cooling the samples to a lower temperature, one or more additional specific binding members can be added to one or more test wells, and, preferably, to a control (or standard) well or wells. The one or more additional specific binding members can comprise or bind particles or beads. Particles and beads that can bind to a target molecule through a specific binding member can increase the size of the target molecule complex, thus providing a larger increase in FP when the fluorophore of the unfolded-specific binding member binds the target.

The one or more additional specific binding members that can bind a particle or bead can specifically bind the target molecule at a site distinct from the binding site recognized by the first specific binding member. In cases where target molecules are proteins, the binding site of the second specific binding member is preferably an attached tag, such as an attached peptide tag, for example, the 6xHis, myc, FLAG, or hemaglutinin tag, or any other short peptide sequence known in the art or later developed that can be specifically recognized by a specific binding member. The use of engineered peptide sequence tags introduced into target proteins allows the use of generic antibody reagents in assays of the present invention, where a generic antibody reagent can be an antibody that recognizes the attached tag and is directly or indirectly coupled to a particle or bead. A generic antibody reagent can be used in assays of the present invention for any target protein that comprises the attached tag recognized by the antibody. The use of an attached tag also avoids the possibility that an additional specific binding member competes with a test compound for binding a particular region of the target molecule or binds an endogenous region of the target protein that is altered during denaturation.

Other strategies for increasing the size of the target molecule-fluorophore complex include the use of polyclonal antibodies that recognize the target protein, the use of secondary antibodies (anti-isotype anti-species antibodies) or selection of conditions at which the unfolded protein aggregates. Binding of multiple antibody molecules to a single target molecule increases the size of the denatured target bound by the fluorophore through the denatured-specific antibody. Polyclonal and secondary antibodies can be added after the denaturation step and prior to detection, to avoid interference with the unfolding process. These strategies can also be used in aspects in which the target molecule is directly labeled with a fluorophore.

The samples are optionally cooled to a temperature of not more than about 37 degrees C. In most cases, assay samples are cooled to approximately room temperature (22 degrees C.). Preferably, where cooling is employed, it occurs at a defined rate. In the alternative, it is also possible to maintain the samples at T_{ATLAS} for the detection step. This requires that the fluorescence polarization detection means can interface with a heating element that can maintain the desired temperature during fluorescence polarization detection.

Fluorescence polarization detection can be performed by any device that can detect fluorescence polarization at the wavelength emitted by the fluorophore used in the assay. Fluorescence detection devices, including those that detect fluorescence from multiwell plates, are known in the art. The fluorescence detection device can interface with the sample heating device, or can be separate.

In preferred aspects of the present invention, at least one control well is made up that lacks a test compound, but that comprises the labeled target in the same amount as the test wells, and the control well is heated and analyzed in the same way and at the same time as the test wells. Preferably, at least one control well is in a multiwell plate that also contains test wells, and the test compound and control assay mixtures are made up at the same time from the same stock concentrations of target molecule, specific binding members, signal molecules, etc.

In the alternative, control wells can be made up at a time other than that when test wells are made up. One or more control wells can be heated and subjected to fluorescence detection measurements, before or after the test wells are heated. The data from the fluorescence detection of a control well can be recorded and stored, such as in a database.

In some aspects of the present invention, one or more standard wells are provided for comparison with one or more test wells. Standard wells comprise target protein and at least one compound that is either a test compound or a compound whose affect on target unfolding is known. One or more standard wells is also heated and analyzed in the same way and preferably at the same time as the test wells. Where standard wells are used to generate a reference value, they can be one, some, or all of the test wells in one or more assays, and can be used to compute an average value of a detection measurement against which individual test well detection measurements can be compared. Preferably, in aspects of the invention in which standard wells are used, at least one standard well is in a multiwell plate that also contains test wells, and the test compound and standard assay mixtures are made up at the same time from the same stock concentrations of target molecule, specific binding members, signal molecules, etc.

In the alternative, standard wells can be made up at a time other than that when test wells are made up. One or more standard wells can be heated and subjected to fluorescence detection measurements, before or after the test wells are heated. The data from the fluorescence detection of a standard well can be recorded and stored, such as in a database.

Determination of Target Molecule Unfolding

Measurements from one or more test wells are compared with measurements from one or more control wells and/or one or more standard wells to determine whether any test compounds significantly alter the fluorescence readout. For example, test wells that differ from control wells by more than a particular amount or percentage in fluorescence polarization at one or more wavelengths, can be identified as wells in which the target molecules has unfolded to a significantly different degree than in control wells lacking test compound.

In most (but not all) cases, the difference in fluorescence signal or signals or determinations based on fluorescence signals will indicate that the test compound has to some degree protected the target molecule from unfolding in response to elevated temperature. When target molecules unfold and allow specific binding members to bind, the fluorescence polarization signal increases due to the longer rotational correlation of the specific binding member bound versus unbound target that comprises a fluorophore. However, it is also possible to identify compounds that promote unfolding of the target under denaturing conditions by detecting a decrease in the fluorescence polarization signal with respect to controls. Compounds that promote unfolding of the target can also be ligands of the target. Without being bound to any particular mechanism, in some cases compound binding may make a target more susceptible to unfolding at a particular temperature.

Identification of Ligands

Test compound wells that differ from control wells by more than a particular amount or percentage in fluorescence polarization can be identified as first screen hits. Those skilled in the art can determine reasonable criteria for identifying first screen hit, such as, for example 20% or greater difference from control data, or preferably a 50% or greater difference from control data.

Preferably, first screen hits are rescreened in the same assay format in which they were originally identified. First screen hits that differ from control wells by more than a particular amount or percentage in fluorescence polarization in a second assay are called duplicate hits.

Duplicate hits can be subjected to a titration series in which they assayed at a range of concentrations. Duplicate hits that are titratable, that is, that show concentration dependency in the assay, are potential ligands for the target molecule. IC 50 values can be determined from these assays.

Test compounds identified as potential target molecule ligands can be tested in other types of assays for independent confirmation of target molecule binding. Examples of such assays are ELISA, filter binding, isothermal calorimetry, or other binding assays as they are known in the art.

High Throughput Screening

The present invention is particularly well-suited to high throughput screening, in which a multiplicity of test compounds can be tested at the same time. Because of the high degree of sensitivity and low background of fluorescence polarization detection, small amounts of protein and correspondingly small volumes can be used for assays. In high throughput assays, samples are preferably made up in wells of multiwell plates. However, other sample containers can be used. For example, the sample containers can be indentations of a surface, or can be capillaries or tubes for holding small volume (sub-milliliter) liquid samples. Preferably, the assay is formatted for high throughput or ultra high throughput screening (HTS or UHTS) involving a multiplicity, and preferably hundreds, of samples, and thus the assays are most conveniently performed in wells of for example, 96, 384, 1536 or 3456 well plates. Plate heating and plate fluorescence detection systems as they are known in the art or designed for the methods of the present invention can be used.

The ATLAS assay can easily be configured such that a minimum of pipeting steps are required. Preferably, liquid handling devices are used for dispensing sample components. In addition, the assay can be performed within a short time period, as assay samples can be assembled, rapidly heated to a single temperature, incubated for less than an hour, rapidly cooled, and detected.

The addition of reagents, as well as heating, incubations, cooling and detection steps can be automated. In a preferred aspect of the present invention, an integrated system employs robotics to dispense reagents, and to move plates comprising test wells to and from dispensing areas, heating/cooling devices, and fluorescence plate readers. Preferably the integrated system is computerized and programmable, and contains software for sample analysis.

III. Methods of Screening Compounds to Identify One or more Target Molecules Logands Using FRET Detection of Aggregates of a Target Molecule

Another embodiment of the present invention is aggregation dependent FRET. The screening methods of aggregation dependent FRET use two members of a FRET pair, in which the FRET partners are integral to or are bound to different molecules of the target protein. In this embodiment, the FRET partners come into proximity when target molecules aggregate. In these methods, soluble aggregates of target proteins that result from denaturation, such as thermal denaturation, are detected by FRET. Target molecules that alter the unfolding of a target molecule and thereby alter the degree of aggregation in the assay are identified by altered FRET in test wells, and are identified as ligands of the target molecule.

In one aspect, the aggregation dependent FRET embodiment includes assays in which at least a portion of the target molecule population to be used in the assay is bound with a first specific binding member, where the first specific binding member comprises or can bind a donor fluorophore or acceptor moiety. One or more aliquots of the target molecule population (a portion of which is bound to the first specific binding member) is contacted with at least one test compound and subjected to denaturing conditions (at which the protein is known to unfold to a measurable extent in the absence of a test compound). After denaturation treatment, a second specific binding member is added. The second specific binding member specifically binds the same single region of the target molecule that is recognized by the first specific binding member. The first and second specific binding members can be the same specific binding member, for example, the same monoclonal antibody, however they are coupled to or can bind different FRET labels. The first and second specific binding members comprise or bind members of FRET pair. That is, in aspects where the first specific binding member binds a FRET donor, the second specific binding member binds a FRET acceptor, and vice versa. Thus when the FRET partners are in proximity, such as when they bind different members of the target molecule population that are aggregated with one another, energy can be transferred from the FRET donor to the FRET acceptor. One or more fluorescence signals is detected, and when compared with fluorescence measurements of a control in which target molecule is subjected to denaturing conditions in the absence of a test compound, or at least one standard in which the target molecule is subjected to denaturing conditions in the presence of a different compound, fluorescence measurement is used as an indicator of the degree to which the target molecule occurs in the unfolded state at the assay conditions. Test compounds that alter the degree to which the target molecule occurs in the unfolded state at the assay conditions can be identified as ligands of a target protein.

In a different aspect of the aggregation dependent FRET embodiment, assays are provided in which at least a portion of a first population of the target molecule to be used in the assay is bound with or comprises a first specific binding member, and at least a portion of a second population of the target molecule to be used in the assay is bound with or comprises a second specific binding member. The first and second specific binding members can each comprise or bind either a FRET donor or a FRET acceptor. Together, the first and second specific binding members comprise or bind members of FRET pair. That is, in aspects where the first specific binding member comprises or can bind a FRET donor, the second specific binding member comprises or can bind a FRET acceptor, and vice versa. The first and second specific binding member-labeled target molecule populations of target molecule are added together to make a mixed first and second specific binding member-labeled population of target molecule. The mixed first and second specific binding member-labeled population of target is contacted with at least one test compound and subjected to denaturing conditions (at which the protein is known to unfold to a measurable extent in the absence of a test compound). After denaturation treatment, soluble aggregates are detected by FRET. Thus when the FRET partners are in proximity, such as when they bind target molecules that are aggregated with one another, energy can be transferred from a FRET donor to a FRET acceptor. One or more fluorescence signals is detected, and when compared with fluorescence measurements of a control in which target protein is subjected to denaturing conditions in the absence of a test compound, the fluorescence measurement is used as an indicator of the degree to which the target molecule occurs in the unfolded state at the assay temperature. Test compounds that alter the degree to which the target molecule occurs in the unfolded state at the assay temperature can be identified as ligands of a target protein.

Methods in which a Portion of a Population of Target Protein is Labeled with a First Specific Binding Member that Can Bind a FRET Partner

A first aspect of aggregation dependent FRET encompasses methods that include: providing a population of a target molecule, in which at least a portion of the population of target protein is labeled with a first specific binding member that can bind a single attached tag of the target molecule, where the first specific binding member comprises or can directly or indirectly bind a FRET donor or a FRET acceptor, and contacting an aliquot of the first specific binding member-labeled target protein with at least one test compound in one or more test wells. The method further includes heating the one or more test wells and at least one control well to a predetermined temperature at which at least a portion of the target molecule is denatured and adding to the one or more test wells and to at least one control well a second specific binding member that can bind the target protein at the single region recognized by the first specific binding member. The second specific binding member comprises or can directly or indirectly bind a FRET donor or FRET acceptor, depending on the nature of the FRET partner attached to the first specific binding member, such that when the first specific binding member comprises or can directly or indirectly bind a FRET donor, the second specific binding member comprises or can directly or indirectly bind a FRET acceptor, and when the first specific binding member comprises or can directly or indirectly bind a FRET acceptor, the second specific binding member comprises or can directly or indirectly bind a FRET donor. The method further includes measuring fluorescence emission at one or more wavelengths from the one or more test wells; making a comparison of fluorescence emission at one or more wavelengths of one or more test wells with a reference value; using said comparison of fluorescence emission to determine the extent to which said target molecule occurs in the unfolded state, the folded state, or both in the test wells; and using the determination of the extent to which said target molecule occurs in the unfolded state, the folded state, or both in the test wells to determine whether one or more test compounds binds the target molecule, thereby identifying one or more ligands of the target molecule.

The target molecule for which ligands are sought can be any molecule, but preferably the target molecule is a biomolecule, more preferably a biomolecule that comprises a peptide, a protein or a nucleic acid, and most preferably a biomolecule that comprises a protein. A biomolecule that comprises a protein or peptide can be, for example, a glycoprotein, lipoprotein, nucleoprotein, or a farnsylated, meristylated, acylated, phosphorylated, or sulfated protein, etc. Where “protein” or “target protein” is used herein, the aforementioned biomolecules that comprise protein are also included.

Target proteins can be of any species origin and can be isolated from native sources, including organisms, environmental sources, or media, or can be produced using recombinant technologies using endogenous or exogenous cell types. For example, target proteins can be produced in bacterial or fungal cultures, insect cell cultures, avian cell cultures, mammalian (including human) cell cultures, etc. They can also be produced by transgenic organisms. The proteins are preferably at least partially purified, and more preferably substantially purified, for use in assays. The proteins can differ in sequence with regard to the native, wild-type form, and can include one or more attached tags.

In preferred aspects of the present invention, a target protein includes an attached tag that can be recognized by a specific binding member, such as a specific binding member that comprises or can bind a label such as a fluorophore or a quencher. In this way generic reagents in the form of primary specific binding members (such as those that can directly or indirectly bind fluorophores or quenchers) that can specifically bind an attached tag can be used in the assays of the present invention. An important advantage of using attached peptide tag sequences is that it avoids the use of a specific binding member that binds an endogenous region of the target protein. Use of an endogenous region is not preferred, since an endogenous region could be a test compound binding site, or could be involved in heat-dependent aggregation of the target protein, or could be a region whose conformation or accessibility changes with sample heating. Examples of attached tags are short peptide “epitope tag” sequences, such as, for example, the FLAG, hemaglutinin, myc, or 6xHis tags. Such peptide epitope tags can be inserted into a target protein sequence using recombinant DNA technology. Preferably, a peptide tag sequence is added to a region of the protein such that it does not disrupt the native structure of a target protein and does not significantly alter the stability of the native structure of a target protein. For example, a peptide sequence tag can be added to the N or C terminus of a target protein. It is critical that where a target molecule comprises an attached tag that is recognized by a specific binding member that comprises or can bind a FRET donor or a FRET acceptor, the attached tag is present only once in the protein. Thus, in this aspect of the present invention a target protein can comprise a single attached tag, such as a peptide tag. Optionally, short peptide linkers can be used to attach a peptide tag sequence to a target protein. Thermal denaturation (assessed by CD or other methods) can be performed with target proteins having engineered peptide epitope tags and the results compared with those of target proteins without engineered tags to determine whether a tag sequence significantly affects the stability of a target protein.

At least a portion of a target protein can be labeled with a first specific binding member in any practical way. For example, a solution of target protein can be mixed with an appropriate amount of antibody, incubated for a period of time, and the labeled protein can optionally be separated from free antibody.

In some aspects of the present invention, it can be desirable to have a fraction of the target protein population labeled with a first specific binding member, where the percentage of target protein population labeled with a first specific binding member any percentage, from less than 1% to more than 90%. In some preferred aspects of the present invention, the fraction of labeled target protein in the target protein population can be about 50%. Assays can be optimized based on the fraction of labeled target protein in the target protein population. Factors such as the donor fluorophore and acceptor moiety used in the assay, the particular target protein, the specific binding members used in the assay, etc. can be factors in determining optimal fractions of labeled target protein. In configurations in which it is desirable to have a fraction of the population labeled, an aliquot of a known amount of target protein can be used in the labeling procedure, and subsequently mixed with an aliquot of unlabeled target protein to generate the desired proportion of labeled target protein in a target protein population to be used in the assays of the present invention.

In some aspects of the present invention, the portion of first specific binding member-labeled target protein in the target protein can be essentially all of the target protein population. “Essentially all” means that all of the target population to be provided in the assay is subjected to the first specific binding member labeling procedure, and the efficiency of the labeling procedure determines the fraction of target protein that is labeled with the first specific binding member. Preferably, in these cases greater than 80% of the target protein is labeled with the first specific binding member, more preferably greater than 90%, and most preferably greater than 95%.

The first specific binding member comprises or can directly or indirectly bind a member of a FRET pair. A first specific binding member can be conjugated to a FRET donor or a FRET acceptor using methods known in the art. Alternatively, the specific binding member can indirectly bind a fluorophore or quencher, for example, through the use of one or more other specific binding member pairs (“secondary specific binding members”). One example of a secondary specific binding member pair that can be used to link a fluorophore or quencher to a primary specific binding member such as an antibody used in the ATLAS assay is biotin-streptavidin. For example, a fluorophore can be linked to streptavidin, and a primary specific binding member used in the assay can be biotinylated (or vice versa). This mechanism of linking a fluorophore (or quencher) to a primary specific binding member such as an antibody can provide flexibility in the assay, such that the fluorophore (or quencher) can optionally be added to the assay mixture at a different time from the addition of the first specific binding member is added (for example, after heating to T_{ATLAS} and subsequent cooling to room temperature, and before signal detection). Other secondary specific binding member pairs that can be used include biotin-avidin, chitin binding domain-chitin binding protein; nitroloacetic acid-6xHis; calmodulin binding domain-calmodulin; etc. It is also possible to use antibodies as secondary specific binding members, for example, isotype- and species-specific secondary antibodies can bind be conjugated to a fluorophore or quencher and can bind primary antibodies used to bind the target protein.

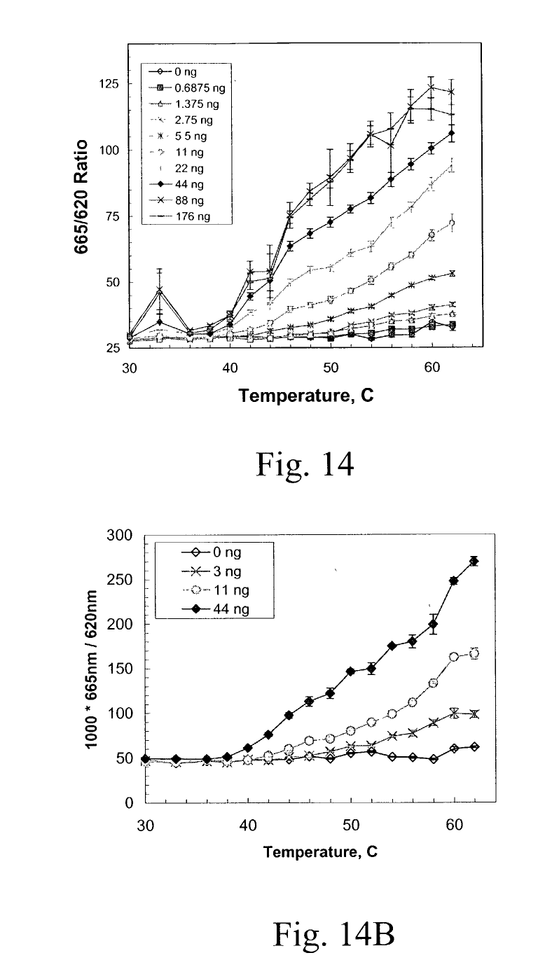

Preferably, a solution of a target molecule is made up, for example in a buffer, and the target molecule solution is added to one or more wells or sample containers. The amount of target molecule used in each sample will vary depending on the target. However, the high sensitivity/low background of the assay using FRET detection allows for very small amounts of target molecule to be used in these assays, for example, where the target molecule is a protein, from about 0.1 ng to 10 microgram, but preferably the amount of target protein in an assay will be in the range of from about 1 ng to 5 micrograms. The optimal amount of a target protein in an assay sample can be determined empirically by titrating the amount of protein in the assay (see, for example, Example 8 and FIG. 14).

One or more test compounds is added to one or more wells or sample containers. Test compounds can be made up in solutions comprising buffers, solvents, or other compounds. Test compounds can be added to one or more wells before, after, or at the same time as target molecules are added to wells. Preferably, test compounds are added to one or more test wells. It is within the scope of the invention to test several concentrations of the test compound in a given assay. It is also within the scope of the present invention to include more than one test compound in a single test well.

More that one test compound can be added to one or more test wells. Preferably, test compounds added to at least two wells are different test compounds, or different amounts or combinations of test compounds. The amount of test compounds introduced into a well can vary, but in many cases will be in the sub-micromolar to micromolar range, such as from about 0.01 micromolar to about 100 micromolar, preferably from about 0.1 micromolar to about 50 micromolar.

Optionally, the target molecule and test compounds (assay mixtures) are incubated for a period of time prior to the heating step. The incubation can be done at any temperature, but, if performed, the pre-incubation is preferably performed at a temperature of not more than 37 degrees C., and more preferably is performed at about 22 degrees C. The pre-heating incubation can be for any length of time, but in cases where it is included, it will typically be for 30 minutes or less.

Preferably, at least one control well comprising the target molecule in the absence of a test compound is included in the assay. Preferably the assay is performed on at least one control well at the same time as the test wells, and all steps of the assay are performed exactly as for the test well or wells; however, it is within the scope of the invention to perform control assays separately, and to record the control data for comparison with test compound assay measurements. One or more measurements from control wells, and values based on measurements from control wells (for example, averages, ratios, anisotropy etc.) whether assayed at the same time as the test wells or not, can be used as a reference value for comparison with one or more test wells.

In the alternative or in addition to including a control well, it is possible to include at least one standard well that comprises a target molecule and at least one compound. The interaction of the compound in the standard well with the target molecule may not be known in advance of the assay, but preferably the degree to which the standard well compound affects denaturation of the target protein is known. In some aspects, standard wells can be test compound wells that are compared with other test compound wells in the assays of the present invention. Preferably, where one or more standard wells is used, the assay is performed on at least one standard well at the same time as the test wells, and all steps of the assay are performed exactly as for the test well or wells; however, it is within the scope of the invention to perform standard assays separately, and to record the standard well data for comparison with test compound assay measurements. One or more measurements from standard wells, and values based on measurements from standard wells (for example, averages, ratios, anisotropy etc.) whether assayed at the same time as the test wells or not, can be used as a reference value for comparison with one or more test wells.

The one or more wells are subjected to conditions at which at least a portion of the target protein is unfolded in the absence of a ligand or test compound. Denaturing conditions can be any conditions that cause loss of secondary, tertiary, or quaternary structure of a target molecule, or alter the three-dimensional conformation of a target molecule, including heat, pH changes, presence of detergents or surfactants, chaotropic agents, salts, chelators, etc. Preferably, the denaturing conditions are elevated temperature and subjecting the test wells to denaturing conditions comprises heating the target molecule and one or more test compounds to one or more predetermined temperatures at which at least a portion of said target molecule is denatured.

In preferred aspects of the present example, the test wells and any control or standard wells will be heated to a single discrete predetermined temperature, termed T_{ATLAS}. T_{ATLAS} can be selected in preliminary experiments in which the target molecule is heated and its degree of unfolding as a function of temperature is monitored (although the identity or any activity of the target molecule need not be known). Preferably, before the assay is performed, the target molecule is characterized to establish a melting (temperature dependent structural unfolding) curve in which a physical measurement that reports on the target molecule's structure is plotted as a function of temperature. The physical measurement can be based on any of a variety of structural determination methods well known in the art, for example, CD, light scattering, UV absorption spectroscopy, differential scanning calorimetry, etc. The melting curve of a target molecule can then used to establish the parameters, including T_{ATLAS} of the assay. Thermal melting can preferably be performed under assay conditions (using buffers, reagents, specific binding members, donor fluorophores, acceptor moieties, and FRET detection that will be used in test compound assays) to obtain a melting curve under assay conditions (in the absence of test compounds) (see Example 8 and FIG. 14). Preferably, T_{ATLAS} will be selected as a temperature at which assay reagents are stable and the assay has a wide dynamic range and high quality (Z′).

In some cases, it may be desirable to heat the wells to more than one discrete temperature (e.g., T_{ATLAS1}, T_{ATLAS2}, etc.), but this is less preferred. This can be desirable in some cases, for example, if melting curves demonstrate that the target molecule has more than one transition temperature that is indicative of unfolding intermediates. Preferably, however, no more than three discrete temperatures are used in the ATLAS assay, and most preferably the wells are heated to a single T_{ATLAS}.

Heating can be performed in any incubator or sample heating device and is preferably performed using a heating device that allows for rapid, uniform, and accurate heating, and preferably cooling, to precise temperatures, as well as accurate temperature maintenance. For example, many commercially available thermocyclers can be used for this purpose. The assay samples can be held at T_{ATLAS} for any period of time, for example from about 3 minutes to about 6 hours, preferably from about 10 minutes to about one hour. However, the time of T_{ATLAS} incubation is not a limitation of the present invention.

The samples are optionally cooled to a temperature less than T_{ATLAS}. In most cases, assay samples are cooled to approximately room temperature (22 degrees C.). Preferably, where cooling is employed, it is relatively rapid and occurs at a defined rate. In the alternative, it is also possible to maintain the samples at T_{ATLAS} for the detection step. This requires that the fluorescence detection means can interface with a heating element that can maintain the desired temperature during fluorescence detection.

After heating to T_{ATLAS}, and preferably, cooling the samples to a lower temperature, a second specific binding member is added to one or more test wells, and, preferably, to a control well or wells. The second specific binding member can comprise or bind a donor fluorophore or an acceptor moiety. The second specific binding member specifically binds the same single region of the target molecule that is recognized by the first specific binding member. By “single region” is meant that the region occurs once and only once in the target molecule. Thus, one specific binding member that recognizes the single attached tag can bind to an individual target molecule. In cases where target molecules are proteins, this single region is preferably an attached tag, such as a short peptide epitope (sometimes referred to as an epitope tag), for example, the 6xHis, myc, FLAG, or hemaglutinin tag, or any other short peptide epitope that can be specifically recognized. The use of a single attached tag introduced into a target proteins allows the use of generic antibody reagents in ATLAS assays, where the generic antibody reagent can be an antibody that recognizes the attached tag and is directly or indirectly coupled to a fluorophore or quencher. The generic antibody reagent can be used in ATLAS for any target protein that has the attached tag. The use of an engineered peptide epitope also avoids the possibility that binding of the single region by specific binding members alters heat-dependent aggregation properties of the target protein, or competes with a test compound for binding a particular region of the target molecule or binds an endogenous region of the target protein that is altered during heat denaturation.

In assays in which the first specific binding member comprises or binds a donor fluorophore, the second specific binding member preferably binds or comprises an acceptor moiety. In assays in which the first specific binding member comprises or binds an acceptor moiety, the second specific binding member preferably binds or comprises a donor fluorophore. Together, the FRET partner bound by the first specific binding member and the FRET partner bound by the second specific binding member make up a FRET pair.

As in the case of the first specific binding member, the second specific binding member used in the assay can be directly or indirectly coupled to the fluorophore or quencher. Direct coupling can be, for example, chemical coupling of the fluorophore through active groups on the specific binding member. Indirect coupling can use secondary specific binding members, such as biotin and streptavidin, that can bind the second specific binding member and the fluorophore, such that the second specific binding member and the fluorophore can be coupled together through biotin-streptavidin binding.

Taken together, the fluorophore that directly or indirectly binds or is integral to the first specific binding member and the fluorophore that directly or indirectly binds or is integral to the second specific binding member form a FRET pair. Nonlimiting examples of FRET pairs that can be useful in the methods of the present invention include terbium/fluorescein, terbium/GFP, terbium/TMR, terbium/Cy3, terbium/R phycoerythrin, Europium/Cy5, Europium/APC, Alexa 488/Alexa 555, Alexa 568/Alexa 647, Alexa 594/Alexa 647, Alexa 647/Alexa 594, Cy3/Cy5, BODIPY FL/BODIPY FL, Fluorescein/TMR, IEDANS/fluorescein, fluorescein/fluorescein, and EDANS/DABCYL. Other FRET pairs comprising a fluorescence donor and an acceptor moiety that are known or become known in the art can also be used. In selecting FRET pairs, donors and acceptors should be chosen in which the donor emission wavelength spectrum overlaps the acceptor absorption wavelength spectrum. In addition, for optimal assay sensitivity, the distance the donor and acceptor will be positioned from each other when both are bound to the target molecule according to the methods of the present invention is preferably less than or equal to the Forster radius of the pair. FRET pairs can be selected based on these criteria (fluorescence spectra and Forster radius values) can be found in the literature (Principles of Fluorescence Spectroscopy, 2^{nd} edition (1999) ed. by Joseph R. Lakowicz, Plenum Publishing Corp.; and literature available from Molecular Probes, Eugene, Oreg. and available at www.probes.com) and tested for their appropriateness and efficacy in assays configured with the test protein thermally melted in the absence of test compound.

The assay further includes detecting fluorescence emission at one or more wavelengths from one or more test wells. The fluorescence emission detected in the assay is the result of the interaction between two FRET partners, either a fluorescence donor and a fluorescence acceptor, or a fluorescence donor and a fluorescence quencher. The assay is configured such that denaturation of a target molecule is detected by its self-aggregation. FRET occurs when specific binding partners that specifically bind the same region of the target molecule are brought into proximity. This occurs when two or more target molecules bind to form an aggregate due to their denaturation. Thus the extent of thermal denaturation of the target molecule determines the intensity or wavelength properties of the fluorescence signal.

The detection of the fluorescence signal can be at one or more wavelengths. For example, the detection of fluorescence can be at the wavelength of the donor fluorophore, where reduced intensity of the fluorescence of the donor fluorophore depends on its proximity to an acceptor fluorophore or quencher. More preferably, the detection of fluorescence can be at the wavelength of an acceptor fluorophore.

Preferably, the detection is fluorescence resonance energy transfer (FRET) detection, where the assay is designed to detect fluorescence of an acceptor fluorophore, and more preferably the assay detects fluorescence of both the donor and the acceptor fluorophore of an acceptor/donor pair. Fluorescence of the donor and acceptor can be expressed as a ratio, for example the ratio of fluorescence at the acceptor emission wavelength to fluorescence at the donor emission wavelength. It is also possible, however, to assay protein unfolding by detecting fluorescence emission at the donor wavelength. For example, fluorescence at the donor wavelength will be reduced by increased protein unfolding as the fluorescence donor can be brought into proximity with a fluorescence acceptor or fluorescence quencher.

Fluorescence detection can be performed by any device that can detect fluorescence at the wavelength emitted by the fluorophore used in the assay. Fluorescence detection devices, including those that detect fluorescence from multiwell plates, are known in the art (for example, Packard Biosciences, Perkin Elmer). The fluorescence detection device can interface with the sample heating device, or can be separate. Preferably, the fluorescence detection device can detect fluorescence at more than one wavelength, and preferably includes software that can calculate a ratio between two wavelength, such as the wavelengths of fluorescence emission of a donor and acceptor used in the assay.

Detection of fluorescence emission at one or more wavelengths is preferably time-resolved fluorescence detection. A preferred detection mechanism used in the methods of the present invention uses time-resolved fluorescence detection at two wavelengths, and thus can be referred to as “time resolved energy transfer” or “TRET”, or “time-resolved fluorescence resonance energy transfer” or “TR-FRET”. TRET (or “TR-FRET”) detection is well known in the art (Pope et al. (1999) Drug Disc Tech 4 (8): 350-362). As practiced in the present invention, TR-FRET involves delaying the measurement of fluorescence intensity at two or more wavelengths by a short time window after excitation of the donor fluorophore. This can reduce the background due to compound interference in fluorescence measurements.

In preferred aspects of the present invention, one or more control wells is made up that lacks a test compound, but that comprises the target molecule and specific binding member(s) in the same amounts as the test wells, and the control well is heated and analyzed in the same way and at the same time as the test wells. Preferably, one or more control wells is in a multiwell plate that also contains test wells, and the test compound and control assay mixtures are made up at the same time from the same stock concentrations of target molecule, specific binding members, signal molecules, etc.

In the alternative, one or more control wells can be made up at a time other than that when test wells are made up. One or more control wells can be heated and subjected to fluorescence detection measurements, before or after the test wells are heated. The data from the fluorescence detection of a control well can be recorded and stored, such as in a database.

In some aspects of the present invention, one or more standard wells are provided for comparison with one or more test wells. Standard wells comprise target protein and at least one compound that is either a test compound or a compound whose affect on target unfolding is known. One or more standard wells is also heated and analyzed in the same way and preferably at the same time as the test wells. Where standard wells are used to generate a reference value, they can be one, some, or all of the test wells in one or more assays, and can be used to compute an average value of a detection measurement against which individual test well detection measurements can be compared. Preferably, in aspects of the invention in which standard wells are used, at least one standard well is in a multiwell plate that also contains test wells, and the test compound and standard assay mixtures are made up at the same time from the same stock concentrations of target molecule, specific binding members, signal molecules, etc.

In the alternative, standard wells can be made up at a time other than that when test wells are made up. One or more standard wells can be heated and subjected to fluorescence detection measurements, before or after the test wells are heated. The data from the fluorescence detection of a standard well can be recorded and stored, such as in a database.

Determination of Target Molecule Unfolding

Measurements from test wells are compared with measurements from one or more control wells or one or more standard wells to determine whether any test compounds significantly alter the fluorescence readout. For example, test wells that differ from control wells by more than a particular amount or percentage in fluorescence intensity at one or more wavelengths, or by more than a particular amount or percentage in a ratio of fluorescence intensity at two or more wavelengths, can be identified as wells in which the target molecules has unfolded to a significantly different degree than in control wells lacking test compound. Test wells that differ from standard wells by more than a particular amount or percentage in fluorescence intensity at one or more wavelengths, or by more than a particular amount or percentage in a ratio of fluorescence intensity at two or more wavelengths, can be identified as wells in which the target molecules has unfolded to a significantly different degree than in standard wells comprising one or more different compounds. The comparison between test compound and control wells or standard wells can be a comparison of fluorescence intensity (or a value derived therefrom) at a fluorescence donor emission wavelength, a comparison of fluorescence intensity (or a value derived therefrom) at a fluorescence acceptor emission wavelength, or a comparison of some value that is a function of both fluorescence donor emission wavelength and fluorescence acceptor emission wavelength. Preferably, where the assay uses a FRET pair comprising a fluorescence donor and a fluorescence acceptor, the comparison is based on a ratio of fluorescence acceptor emission to fluorescence donor emission. Preferably, where the assay uses a FRET pair comprising a fluorescence donor and a fluorescence quencher, the comparison is based on donor wavelength emission intensities.

In most (but not all) cases, a significant difference in fluorescence signal or signals or determinations based on fluorescence signals will indicate that a test compound has to some degree protected the target molecule from unfolding in response to denaturing conditions such as elevated temperature. In the case of a fluorescence donor/fluorescence acceptor pair, a reduction in the ratio of acceptor to donor fluorescence is indicative of a reduction in target unfolding and subsequent aggregation in the presence of test compound. In the case of a fluorescence donor/fluorescence quencher pair, an increase in the intensity of donor fluorescence is indicative of a reduction in target unfolding and subsequent aggregation in the presence of test compound. It is also possible to identify compounds that promote unfolding of the target by detecting an increase in the ratio of acceptor to donor fluorescence or, in the case of a donor/quencher pair, a decrease in the intensity of donor fluorescence. Compounds that promote unfolding of the target can also be ligands of the target. Without being bound to a particular mechanism, in some cases compound binding may make a target more susceptible to unfolding at a particular temperature.

Identification of Ligands

Test wells that differ from control wells by more than a particular amount or percentage in fluorescence intensity at one or more wavelengths, or by more than a particular amount or percentage in a ratio of fluorescence intensity at two or more wavelengths, can be identified as wells that comprise test compounds that protect the target molecule from unfolding at elevated temperature. Test compounds identified as stabilizing the target molecule at high temperature are identified as ligands of the target molecule. Those skilled in the art can determine reasonable criteria for identifying first screen ligands, such as, for example 20% or greater difference from control data, or preferably a 50% or greater difference from control data.

Preferably, first screen hits are rescreened in the same assay format in which they were originally identified. First screen hits that differ from control wells by more than a particular amount or percentage in fluorescence intensity at one or more wavelengths, or by more than a particular amount or percentage in a ratio of fluorescence intensity at two or more wavelengths, in a second assay are called duplicate hits.

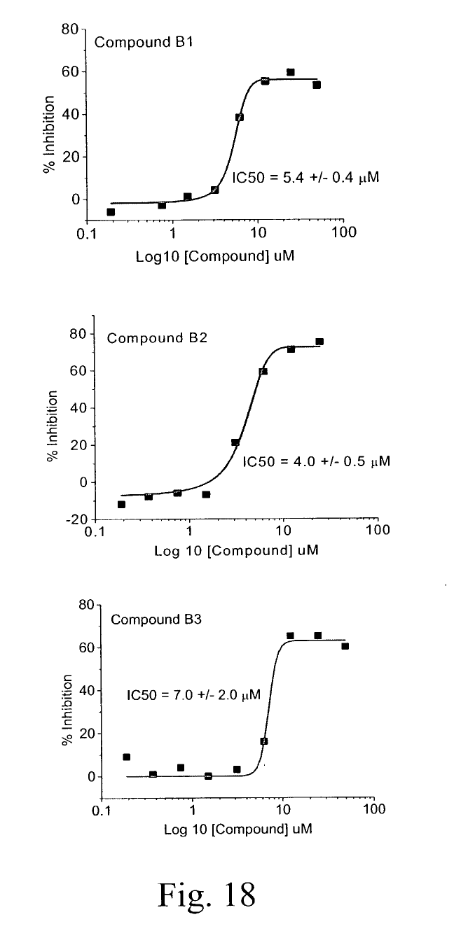

Duplicate hits can be subjected to a titration series in which they assayed at a range of concentrations (see Example 11 and FIG. 18). Duplicate hits that are titratable, that is, that show concentration dependency in the assay, are considered ligands for the target molecule. IC 50 values can be determined from these assays.

Test compounds identified as target molecule ligands can optionally be tested in other types of assays for independent confirmation of target molecule binding. Examples of such assays are ELISA, filter binding, isothermal temperature calorimetry, or other binding assays as they are known in the art.

High Throughput Screening

The present invention is particularly well-suited to high throughput screening, in which a multiplicity of test compounds can be tested at the same time. Because of the high degree of sensitiviy and low background of FRET detection, and particulary TR-FRET detection, small amounts of protein and correspondingly small volumes can be used for assays. In high throughput assays, samples are preferably made up in wells of multiwell plates. However, other sample containers can be used. For example, the sample containers can be indentations of a surface, or can be capillaries or tubes for holding small volume (sub-milliliter) liquid samples. Preferably, the assay is formatted for high throughput or ultra high throughput screening (HTS or UHTS) involving a multiplicity, and preferably hundreds, of samples, and thus the assays are most conveniently performed in wells of for example, 96, 384, 1536, or 3456 well plates. Plate heating and plate fluorescence detection systems as they are known in the art or designed for the methods of the present invention can be used.

The ATLAS assay can easily be configured such that a minimum of pipeting steps are required. In addition, the assay can be performed within a short time period, as assay samples can be assembled, rapidly heated to a single temperature, incubated for less than an hour, rapidly cooled, and detected.

The addition of reagents, as well as heating, incubations, cooling and detection steps can be automated. In a preferred aspect of the present invention, an integrated system employs robotics to dispense reagents, and to move plates comprising test wells to and from dispensing areas, heating/cooling devices, and fluorescence plate readers. Preferably the integrated system is computerized and programmable, and contains software for sample analysis.

Methods in which One Population of a Target Protein is Labeled with a First FRET Partner, and a Second Population of Target Protein is Labeled with a Second FRET Partner

The aggregation dependent FRET embodiment of the present invention also encompasses methods that include: providing a first population of a target molecule, in which the first population is labeled with or can bind a donor fluorophore or acceptor moiety; adding to the first population of target molecule a second population of target molecule in which the second population is labeled with or can bind an acceptor moiety; to form a mixed donor/acceptor population of target molecule. The method further includes contacting the mixed donor/acceptor population of target molecule with one or more test compounds in one or more test wells and heating the one or more test wells to a predetermined temperature at which at least a portion of the target molecule is denatured. The method further includes measuring fluorescence emission at one or more wavelengths from the one or more test wells; making a comparison of fluorescence emission at one or more wavelengths of one or more test wells with a reference value; using said comparison of fluorescence emission to determine the extent to which said target molecule occurs in the unfolded state, the folded state, or both in the wells comprising target molecules and test compounds; and using the determination of the extent to which said target molecule occurs in the unfolded state, the folded state, or both in the one or more test wells to determine whether one or more test compounds binds said target molecule, thereby identifying one or more ligands of said target molecule.

The target molecule for which ligands are sought can be any molecule, but preferably the target molecule is a biomolecule, more preferably a biomolecule that comprises a peptide, a protein or a nucleic acid, and most preferably a biomolecule that comprises a protein. A biomolecule that comprises a protein or peptide can be, for example, a glycoprotein, lipoprotein, nucleoprotein, or a farnsylated, meristylated, acylated, phosphorylated, or sulfated protein, etc. Where “protein” or “target protein” is used herein, the aforementioned biomolecules that comprise protein are also included.

Target proteins can be of any species origin and can be isolated from native sources, including organisms, environmental sources, or media, or can be produced using recombinant technologies using endogenous or exogenous cell types. For example, target proteins can be produced in bacterial or fungal cultures, insect cell cultures, avian cell cultures, mammalian (including human) cell cultures, etc. They can also be produced by transgenic organisms. The proteins are preferably at least partially purified, and more preferably substantially purified, for use in assays. The proteins can differ in sequence with regard to the native wild-type form, and can optionally include one or more attached tags.

A target protein can optionally include an attached tag that can be recognized by a specific binding member, such as a specific binding member that comprises or can bind a label such as a fluorophore. In this way generic reagents in the form of primary specific binding members (such as those that can directly or indirectly bind fluorophores or quenchers) that can specifically bind an attached tag can be used in the assays of the present invention. An important advantage of using an attached tag (such as a small peptide epitope) is that it avoids the use of a specific binding member that binds an endogenous region of the target protein. Use of an endogenous region is not preferred, since an endogenous region could comprise a test compound binding site, or could be involved in heat-dependent aggregation of the target protein, or could be a region whose conformation or accessibility changes with sample heating. Examples of attached tags are short peptide epitope “tag” sequences, such as, for example, the FLAG, hemagglutinin, myc, or 6xHis tags. Such tag sequences can be inserted into a target protein sequence using recombinant DNA technology. Preferably, a peptide epitope tag is added to a region of the protein such that it does not disrupt the native structure of a target protein and does not significantly alter the stability of the native structure of a target protein. For example, a peptide sequence tag can be added to the N or C terminus of a target protein. Optionally, short peptide linkers can be used to attach a tag sequence to a target protein. Thermal denaturation (assessed by CD or other methods) can be performed with target proteins having tags and the results compared with those of target proteins without tags to determine whether a tag sequence significantly affects the stability of a target protein.

Target molecules of the first and second populations comprise or can directly or indirectly bind a donor fluorophore or acceptor moiety. A variety of strategies can be used to label target molecules of the first and second populations, where variables can include the types of fluorophores or quenchers used to label the target molecules, whether the labels are integral to or directly or indirectly bound to the target molecules, and at what point in the assay procedure fluorophores or quenchers are bound to target molecules. In configuring the assay however, it is preferable that: 1) in assays in which members of the first target molecule population binds a donor fluorophore, members of the second target molecule population bind an acceptor moiety, and in assays in which members of the first target molecule population bind an acceptor moiety, members of the second target molecule population bind a donor fluorophore; 2) taken together, members of the first and second populations of the target molecule comprise or bind donor fluorophores and acceptor moieties that make up a FRET pair; and 3) donor fluorophores and acceptor moieties used in the assay are added at some point prior to the detection step.

For example, target molecules of the first population can be chemically coupled to a FRET donor and target molecules of the second population can be chemically coupled to a FRET acceptor, or vice versa, prior to adding the first population to the second population. Alternatively, the first and second populations of target molecule can each be bound to specific binding members that are coupled to FRET partners prior to combining the populations. Various combinations of ways of labeling the first and second populations with FRET partners are possible.

For example, the assay can be configured such that only the first population of target molecules comprises an engineered tag sequence that can be recognized by a specific binding member that binds a member of a FRET pair. In this case, the second population of target molecules can be chemically coupled to a FRET partner. Alternatively, the first population can comprise one engineered tag sequence, and the second population can comprise a different engineered tag sequence. The two different tag sequences can be recognized by two different antibodies that are coupled to two different members of a FRET pair. In yet another alternative, either the first or the second population of target molecules can be biotinylated, and can be bound by, for example, a FRET donor or acceptor linked to streptavidin prior to detection.

The second population of target molecules is added to the first population of target molecules to make a mixed population of target molecules. The two populations can be combined at any ratio, but typically will be combined at about a 1:1 ratio. Preferably, the mixed population of target molecules is made up, for example in a buffer. The amount of target molecule used in each sample will vary from target to target. However, the high sensitivity/low background of the assay using FRET detection allows for very small amounts of target molecule to be used in these assays, for example, where the target molecule is a protein, from about 0.1 ng to 10 micrograms, but preferably the amount of target protein in an assay will be in the range of from about 1 ng to 5 micrograms. The optimal amount of a target protein in an assay sample can be determined empirically by titrating the amount of protein in the assay.

One or more test compounds is added to at least one well or sample container. Test compounds can be made up in solutions comprising buffers, solvents, or other compounds. Test compounds can be added to one or more wells before, after, or at the same time as target molecules are added to wells. Preferably, test compounds are added to a plurality of wells. It is within the scope of the invention to test several concentrations of the test compound in a given assay. It is also within the scope of the present invention to include more than one test compound in a single test well.

More that one test compound can be added to one or more wells. Preferably, test compounds added to at least two wells are different test compounds, or different amounts or combinations of test compounds. The amount of test compounds introduced into a well can vary, but in many cases will be in the sub-micromolar to micromolar range, such as from about 0.01 micromolar to about 100 micromolar, preferably from about 0.1 micromolar to about 50 micromolar.

Optionally, the mixed population of target molecules and test compounds (assay mixtures) are incubated for a period of time prior to the heating step. The incubation can be done at any temperature, but, if performed, the pre-incubation is preferably performed at a temperature of not more than 37 degrees C., and more preferably is performed at about 22 degrees C. The pre-heating incubation can be for any length of time, but in cases where it is included, it will typically be for 30 minutes or less.

Preferably, at least one control well comprising the target molecule in the absence of a test compound is included in the assay. Preferably the assay is performed on at least one control well at the same time as the test wells, and all steps of the assay are performed exactly as for the test well or wells; however, it is within the scope of the invention to perform control assays separately, and to record the control data for comparison with test compound assay measurements. One or more measurements from control wells, and values based on measurements from control wells (for example, averages, ratios, anisotropy etc.) whether assayed at the same time as the test wells or not, can be used as a reference value for comparison with one or more test wells.

In the alternative or in addition to including a control well, it is possible to include at least one standard well that comprises a target molecule and at least one compound. The interaction of the compound in the standard well with the target molecule may not be known in advance of the assay, but preferably the degree to which the standard well compound affects denaturation of the target protein is known. In some aspects, standard wells can be test compound wells that are compared with other test compound wells in the assays of the present invention. Preferably, where one or more standard wells is used, the assay is performed on at least one standard well at the same time as the test wells, and all steps of the assay are performed exactly as for the test well or wells; however, it is within the scope of the invention to perform standard assays separately, and to record the standard well data for comparison with test compound assay measurements. One or more measurements from standard wells, and values based on measurements from standard wells (for example, averages, ratios, anisotropy etc.) whether assayed at the same time as the test wells or not, can be used as a reference value for comparison with one or more test wells.

The one or more wells are subjected to conditions at which at least a portion of the target protein is unfolded in the absence of a ligand or test compound. Denaturing conditions can be any conditions that cause loss of secondary, tertiary, or quaternary structure of a target molecule, or alter the three-dimensional conformation of a target molecule, including heat, pH changes, presence of detergents or surfactants, chaotropic agents, salts, chelators, etc. Preferably, the denaturing conditions are elevated temperature and subjecting the test wells to denaturing conditions comprises heating the target molecule and one or more test compounds to one or more predetermined temperatures at which at least a portion of said target molecule is denatured.

In preferred aspects of the present example, the test wells and any control or standard wells will be heated to a single discrete predetermined temperature, termed T_{ATLAS}. T_{ATLAS} can be selected in preliminary experiments in which the target molecule is heated and its degree of unfolding as a function of temperature is monitored (although the identity or any activity of the target molecule need not be known). Preferably, before the assay is performed, the target molecule is characterized to establish a melting (temperature dependent structural unfolding) curve in which a physical measurement that reports on the target molecule's structure is plotted as a function of temperature. The physical measurement can be based on any of a variety of structural determination methods well known in the art, for example, CD, light scattering, UV absorption spectroscopy, differential scanning calorimetry, etc. The melting curve of a target molecule can then used to establish the parameters, including T_{ATLAS} of the assay. Thermal melting can preferably be performed under assay conditions (using buffers, reagents, specific binding members, donor fluorophores, acceptor moieties, and FRET detection that will be used in test compound assays) to obtain a melting curve under assay conditions (in the absence of test compounds) (see Example 8 and FIG. 14). Preferably, T_{ATLAS} will be selected as a temperature at which assay reagents are stable and the assay has a wide dynamic range and high quality (Z′).

In some cases, it may be desirable to heat the wells to more than one discrete temperature (e.g., T_{ATLAS1}, T_{ATLAS2}, etc.), but this is less preferred. This can be desirable in some cases, for example, if melting curves demonstrate that the target molecule has more than one transition temperature that is indicative of unfolding intermediates. Preferably, however, no more than three discrete temperatures are used in the ATLAS assay, and most preferably the wells are heated to a single T_{ATLAS}.

Heating can be performed in any incubator or sample heating device and is preferably performed using a heating device that allows for rapid, uniform, and accurate heating, and preferably cooling, to precise temperatures, as well as accurate temperature maintenance. For example, many commercially available thermocyclers can be used for this purpose. The assay samples can be held at T_{ATLAS} for any period of time, for example from about 3 minutes to about 6 hours, preferably from about 10 minutes to about one hour. However, the time of T_{ATLAS} incubation is not a limitation of the present invention.

The samples are optionally cooled to a temperature less than T_{ATLAS}. In most cases, assay samples are cooled to approximately room temperature (22 degrees C.). Preferably, where cooling is employed, it is relatively rapid and occurs at a defined rate. In the alternative, it is also possible to maintain the samples at T_{ATLAS} for the detection step. This requires that the fluorescence detection means can interface with a heating element that can maintain the desired temperature during fluorescence detection.

Depending on how the assay is configured, one or more specific binding members or FRET partners may be added to the one or more test wells, and, preferably, to a control well or wells after heating to T_{ATLAS}, and preferably, cooling the samples to a lower temperature. For example, the first population in the mixed population of target molecules can be bound to an a first antibody that is biotinylated and that recognizes an attached tag of the target molecule, and the second population in the mixed population of target molecules can be bound to an a second antibody that is directly linked to a fluorescence donor. Prior to detection, an antigen linked FRET acceptor moiety can be added to the wells for labeling of the first population. In an alternative configuration, the first and second target molecule populations each comprise a distinct attached tag (for example, the first population comprises a 6xHis tag and the second population comprises a FLAG tag). Antibodies that recognize the 6xHis tag coupled to a fluorescence donor and antibodies that recognize the FLAG tag coupled to an acceptor moiety can be added in a “Revelation Mix” after heating of the samples and prior to fluorescence detection. In certain cases, the addition of specific binding members after the samples have been brought to a temperature below T_{ATLAS} and before fluorescence detection can obviate problems of heat sensitivity of some antibodies. In some cases, the addition of fluorophores (and, optionally, quenchers) after the samples have been brought to a temperature below T_{ATLAS} and before fluorescence detection can avoid the possibility of interference of a fluorophore with unfolding of the target molecule, and can avoid potential problems due to heat-instability of fluorophores.

In assays in which two different specific binding members are used, the first specific binding member that binds the first population of target molecules comprises or binds a donor fluorophore, the second specific binding member that binds the second population of target molecules preferably binds or comprises an acceptor moiety. In assays in which the first specific binding member comprises or binds an acceptor moiety, the second specific binding member preferably binds or comprises a donor fluorophore. The specific binding members used in the assay can be directly or indirectly coupled to the fluorophore or quencher. Direct coupling can be, for example, chemical coupling of the fluorophore through active groups on the specific binding member. Indirect coupling can use further secondary specific binding members, such as biotin and streptavidin, that can bind the second specific binding member and the fluorophore, such that the second specific binding member and the fluorophore can be coupled together through biotin-streptavidin binding.

Taken together, the fluorophore that directly or indirectly binds or is integral to the first specific binding member and the fluorophore that directly or indirectly binds or is integral to the second specific binding member form a FRET pair. Nonlimiting examples of FRET pairs that can be useful in the methods of the present invention include terbium/fluorescein, terbium/GFP, terbium/TMR, terbium/Cy3, terbium/R phycoerythrin, Europium/Cy5, Europium/APC, Alexa 488/Alexa 555, Alexa 568/Alexa 647, Alexa 594/Alexa 647, Alexa 647/Alexa 594, Cy3/Cy5, BODIPY FL/BODIPY FL, Fluorescein/TMR, IEDANS/fluorescein, fluorescein/fluorescein, and EDANS/DABCYL. Other FRET pairs comprising a fluorescence donor and an acceptor moiety that are known or become known in the art can also be used. In selecting FRET pairs, donors and acceptors should be chosen in which the donor emission wavelength spectrum overlaps the acceptor absorption wavelength spectrum. In addition, for optimal assay sensitivity, the distance the donor and acceptor will be positioned from each other when both are bound to the target molecule according to the methods of the present invention is preferably less than or equal to the Forster radius of the pair. FRET pairs can be selected based on these criteria (fluorescence spectra and Forster radius values) can be found in the literature (Principles of Fluorescence Spectroscopy, 2^{nd} edition (1999) ed. by Joseph R. Lakowicz, Plenum Publishing Corp.; and literature available from Molecular Probes, Eugene, Oreg. and available at www.probes.com ) and tested, for their appropriateness and efficacy in assays configured with the test protein thermally melted in the absence of test compound.

The ATLAS assay further includes detecting fluorescence emission at one or more wavelengths from one or more test wells. The fluorescence emission detected in the ATLAS assay is the result of the interaction between two FRET partners, either a fluorescence donor and a fluorescence acceptor, or a fluorescence donor and a fluorescence quencher. The assay is configured such that denaturation of a target molecule is detected by its self-aggregation in solution. FRET occurs when specific binding partners that specifically bind the same region of the target molecule are brought into proximity. Thus the extent of thermal denaturation of the target molecule determines the intensity or wavelength properties of the fluorescence signal.

The detection of the fluorescence signal can be at one or more wavelengths. For example, the detection of fluorescence can be at the wavelength of the donor fluorophore, where reduced intensity of the fluorescence of the donor fluorophore depends on its proximity to an acceptor fluorophore or quencher. More preferably, the detection of fluorescence can be at the wavelength of an acceptor fluorophore.

Preferably, the detection is fluorescence resonance energy transfer (FRET) detection, where the assay is designed to detect fluorescence of an acceptor fluorophore, and more preferably the assay detects fluorescence of both the donor and the acceptor fluorophore of an acceptor/donor pair. Fluorescence of the donor and acceptor can be expressed as a ratio, for example the ratio of fluorescence at the acceptor emission wavelength to fluorescence at the donor emission wavelength. It is also possible, however, to assay protein unfolding by detecting fluorescence emission at the donor wavelength. For example, fluorescence at the donor wavelength will be reduced by increased protein unfolding as the fluorescence donor can be brought into proximity with a fluorescence acceptor or fluorescence quencher.

Fluorescence detection can be performed by any device that can detect fluorescence at the wavelength emitted by the fluorophore used in the assay. Fluorescence detection devices, including those that detect fluorescence from multiwell plates, are known in the art (for example the Victor V manufactured by Perkin Elmer and the Fusion analyzer manufactured by Packard Biosciences). The fluorescence detection device can interface with the heating device, or can be separate. Preferably, the fluorescence detection device can detect fluorescence at more than one wavelength, and preferably includes software that can calculate a ratio between two wavelength, such as the wavelengths of fluorescence emission of a donor and acceptor used in the assay.

Detection of fluorescence emission at one or more wavelengths is preferably time-resolved fluorescence detection. A preferred detection mechanism used in the methods of the present invention uses time-resolved fluorescence detection at two wavelengths, and thus can be referred to as “time resolved energy transfer” or “TRET”, or “time-resolved fluorescence resonance energy transfer” or “TR-FRET”. TRET (or “TR-FRET”) detection is well known in the art (Pope et al. (1999) Drug Disc Tech 4 (8): 350-362). As practiced in the present invention, TR-FRET involves delaying the measurement of fluorescence intensity at two or more wavelengths by a short time window after excitation of the donor fluorophore. This can reduce the background due to compound interference in fluorescence measurements.

Detection of fluorescence emission at one or more wavelengths is preferably time-resolved fluorescence detection. A preferred detection mechanism used in the methods of the present invention uses time-resolved fluorescence detection at two wavelengths, and thus can be referred to as “time resolved energy transfer” or “TRET”, or “time-resolved fluorescence resonance energy transfer” or “TR-FRET”. TRET (or “TR-FRET”) detection is well known in the art (Pope et al. (1999) Drug Disc Tech 4 (8): 350-362). As practiced in the present invention, TR-FRET involves delaying the measurement of fluorescence intensity at two or more wavelengths by a short time window after excitation of the donor fluorophore. This can reduce the background due to compound interference in fluorescence measurements.

In preferred aspects of the present invention, one or more control wells is made up that lacks a test compound, but that comprises the target molecule and specific binding member(s) in the same amounts as the test wells, and the control well is heated and analyzed in the same way and at the same time as the test wells. Preferably, one or more control wells is in a multiwell plate that also contains test wells, and the test compound and control assay mixtures are made up at the same time from the same stock concentrations of target molecule, specific binding members, signal molecules, etc.

In the alternative, one or more control wells can be made up at a time other than that when test wells are made up. One or more control wells can be heated and subjected to fluorescence detection measurements, before or after the test wells are heated. The data from the fluorescence detection of a control well can be recorded and stored, such as in a database.

In some aspects of the present invention, one or more standard wells are provided for comparison with one or more test wells. Standard wells comprise target protein and at least one compound that is either a test compound or a compound whose affect on target unfolding is known. One or more standard wells is also heated and analyzed in the same way and preferably at the same time as the test wells. Where standard wells are used to generate a reference value, they can be one, some, or all of the test wells in one or more assays, and can be used to compute an average value of a detection measurement against which individual test well detection measurements can be compared. Preferably, in aspects of the invention in which standard wells are used, at least one standard well is in a multiwell plate that also contains test wells, and the test compound and standard assay mixtures are made up at the same time from the same stock concentrations of target molecule, specific binding members, signal molecules, etc.

In the alternative, standard wells can be made up at a time other than that when test wells are made up. One or more standard wells can be heated and subjected to fluorescence detection measurements, before or after the test wells are heated. The data from the fluorescence detection of a standard well can be recorded and stored, such as in a database.

Determination of Target Molecule Unfolding

Measurements from one or more test wells are compared with measurements from at least one control wells to determine whether any test compounds significantly alter the fluorescence readout. For example, test wells that differ from control wells by more than a particular amount or percentage in fluorescence intensity at one or more wavelengths, or by more than a particular amount or percentage in a ratio of fluorescence intensity at two or more wavelengths, can be identified as wells in which the target molecules has unfolded to a significantly different degree than in control wells lacking test compound. The comparison between test and control wells can be a comparison of fluorescence intensity (or a value derived therefrom) at a fluorescence donor emission wavelength, a comparison of fluorescence intensity (or a value derived therefrom) at a fluorescence acceptor emission wavelength, or a comparison of some value that is a function of both fluorescence donor emission wavelength and fluorescence acceptor emission wavelength. Preferably, where the assay uses a FRET pair comprising a fluorescence donor and a fluorescence acceptor, the comparison is based on a ratio of time-resolved fluorescence acceptor emission to fluorescence donor emission. Preferably, where the assay uses a FRET pair comprising a fluorescence donor and a fluorescence quencher, the comparison is based on time-resolved donor wavelength emission intensities.

In most (but not all) cases, the difference in fluorescence signal or signals or determinations based on fluorescence signals will indicate that the test compound has to some degree protected the target molecule from unfolding in response to elevated temperature. In the case of a fluorescence donor/fluorescence acceptor pair, a reduction in the ratio of acceptor to, donor fluorescence is indicative of a reduction in target unfolding in the presence of test compound. In the case of a fluorescence donor/fluorescence quencher pair, an increase in the intensity of donor fluorescence is indicative of a reduction in target unfolding in the presence of test compound. Compounds that promote unfolding of the target can also be ligands of the target. Without being bound to a particular mechanism, in some cases compound binding may make a target more susceptible to unfolding at a particular temperature.

Identification of Ligands

Test wells that differ from control wells by more than a particular amount or percentage in fluorescence intensity at one or more wavelengths, or by more than a particular amount or percentage in a ratio of fluorescence intensity at two or more wavelengths, can be identified as first screen hits. Those skilled in the art can determine reasonable criteria for identifying first screen hit, such as, for example 20% or greater difference from control data, or preferably a 50% or greater difference from control data.

Preferably, first screen hits are rescreened in the same assay format in which they were originally identified. First screen hits that differ from control wells by more than a particular amount or percentage in fluorescence intensity at one or more wavelengths, or by more than a particular amount or percentage in a ratio of fluorescence intensity at two or more wavelengths, in a second assay are called duplicate hits.

Duplicate hits can be subjected to a titration series in which they assayed at a range of concentrations. Duplicate hits that are titratable, that is, that show concentration dependency in the assay, are potential ligands for the target molecule. IC 50 values can be determined from these assays.

Test compounds identified as target molecule ligands can be tested in other types of assays for independent confirmation of target molecule binding. Examples of such assays are ELISA, filter binding, isothermal calorimetry, or other binding assays as they are known in the art.

High Throughput Screening

The present invention is particularly well-suited to high throughput screening, in which a multiplicity of test compounds can be tested at the same time. Because of the high degree of sensitiviy and low background of FRET detection, and particulary TR-FRET detection, small amounts of protein and correspondingly small volumes can be used for assays. In high throughput assays, samples are preferably made up in wells of multiwell plates. However, other sample containers can be used. For example, the sample containers can be indentations of a surface, or can be capillaries or tubes for holding small volume (sub-milliliter) liquid samples. Preferably, the assay is formatted for high throughput or ultra high throughput screening (HTS or UHTS) involving a multiplicity, and preferably hundreds, of samples, and thus the assays are most conveniently performed in wells of for example, 96, 384, 1536, or 3456 well plates. Plate heating and plate fluorescence detection systems as they are known in the art or designed for the methods of the present invention can be used.

The ATLAS assay can easily be configured such that a minimum of pipeting steps are required. For example, two or three reagent mixes can be used: one containing test compound, one containing two populations of target protein, and optionally one containing the “revelation mix” of fluorophores, secondary specific binding members, and a second specific binding member. Preferably, liquid handling devices are used for dispensing sample components. In addition, the assay can be performed within a short time period, as assay samples can be assembled, rapidly heated to a single temperature, incubated for less than an hour, rapidly to cooled, and detected.

The addition of reagents, as well as heating, incubations, cooling and detection steps can be automated. In a preferred aspect of the present invention, an integrated system employs robotics to dispense reagents, and to move plates comprising test wells to and from dispensing areas, heating/cooling devices, and fluorescence plate readers. Preferably the integrated system is computerized and programmable, and contains software for sample analysis.

IV. Methods of Screening Compounds to Identify One or more Ligands that Bind to a Target Molecule by Detecting Aggregates by Fluorescence Polarization

One embodiment of the present invention is screening methods for identifying one or more ligands of a target molecule in which the screening methods use a target molecule labeled with a fluorophore and fluorescence polarization detection as a measure of target unfolding. A portion, preferably but optionally a small percentage, of a population of a target molecule to be used in the assay is directly or indirectly bound to a fluorophore to generate a “doped” target molecule population. The target molecule population is then contacted with at least one test compound and heated to one or more predetermined assay temperatures (at which the protein is known to unfold to a measurable extent in the absence of a test compound). Unfolding of the target molecule in response to heating causes it to aggregate in solution. Soluble aggregates of the fluorescently labeled target protein will have a higher degree of fluorescence polarization than will unaggregated target protein. The use of a doped population in which only a small percentage of target protein is labeled greatly reduces the potential for artifacts in thermal stability and aggregation behavior due to the bound labeling compound. After heating, fluorescence polarization is detected, and when compared with fluorescence polarization measurements of a control in which labeled target protein is heated in the absence of a test compound, the fluorescence polarization measurement is used as an indicator of the degree to which the target molecule occurs in the unfolded state at the assay temperature. In this “doped aggregation fluorescence polarization” (DAFP) assay, test compounds that reduce the degree to which the target molecule occurs in the unfolded state at the assay temperature are identified as potential ligands of a target protein.

The method includes: providing a population of a target molecule, at least a portion of which comprises or is bound to a fluorophore; contacting an aliquot of the population of target molecule with one or more test compounds in one or more test wells; and subjecting the one or more test wells to conditions at which at least a portion of the target molecule is denatured. The method further includes: measuring fluorescence polarization from the one or more test wells and from at least one control well; making a comparison of fluorescence polarization values of one or more test wells with a fluorescence polarization reference value; using said comparison of fluorescence polarization values to determine the extent to which said target molecule occurs in the unfolded state, the folded state, or both in the wells comprising target molecules and test compounds; and using the determination of the extent to which said target molecule occurs in the unfolded state, the folded state, or both in the wells comprising target molecules and test compounds to determine whether one or more test compounds binds said target molecule, thereby identifying one or more ligands of said target molecule.

The target molecule for which ligands are sought can be any molecule, but preferably the target molecule is a biomolecule, more preferably a biomolecule that comprises a peptide, a protein or a nucleic acid, and most preferably a biomolecule that comprises a protein. A biomolecule that comprises a protein can be, for example, a glycoprotein, lipoprotein, nucleoprotein, or a farnsylated, meristylated, acylated, phosphorylated, or sulfated protein, etc. Where “protein” or “target protein” is used herein, the aforementioned biomolecules that comprise protein are also included.

Target proteins can be of any species origin and can be isolated from native sources, including organisms, environmental sources, or media, or can be produced using recombinant technologies using endogenous or exogenous cell types. For example, target proteins can be produced in bacterial or fungal cultures, insect cell cultures, avian cell cultures, mammalian (including human) cell cultures, etc. They can also be produced by transgenic organisms. The proteins are preferably at least partially purified, and more preferably substantially purified, for use in assays. The proteins can differ in sequence with regard to the native wild-type form, and can include one or more attached tags.

A target protein can optionally include an attached tag that can be recognized by a specific binding member, such as a specific binding member that comprises or can bind a label such as a fluorophore. In this way generic reagents in the form of primary specific binding members (such as those that can directly or indirectly bind fluorophores) that can specifically bind an attached tag can be used in the assays of the present invention. An important advantage of using engineered peptide tag sequences is that it avoids the use of a specific binding member that binds an endogenous region of the target protein. Use of an endogenous region is not preferred, since an endogenous region could be a test compound binding site, or could be involved in heat-dependent aggregation of the target protein, or could be a region whose conformation or accessibility changes with sample heating. Examples of attached tags are short peptide “tag” sequences, such as, for example, the FLAG, hemagglutinin, myc, or 6xHis tags. Such tags can be inserted into a target protein sequence using recombinant DNA technology. Preferably, a peptide tag is added to a region of the protein such that it does not disrupt the native structure of a target protein and does not significantly alter the stability of the native structure of a target protein. For example, a peptide sequence tag can be added to the N or C terminus of a target protein. Optionally, short peptide linkers can be used to attach a tag sequence to a target protein. Thermal denaturation (assessed by CD or other methods) can be performed with target proteins having tags and the results compared with those of target proteins without tags to determine whether a tag sequence significantly affects the stability of a target protein.

At least a portion of a population of a target molecule used in the methods of the present invention is labeled with a fluorophore. Preferably, the percentage of the target population that is labeled with a fluorophore is small, for example less than 5%, preferably less than 1%, more preferably less than 0.5%, and most preferably about 0.1% or less. A small percentage of labeled target molecules in the population to be assayed greatly reduces the chance of introducing artifacts due to the effects of label. Labeling a small percentage of a target molecule population can be done by labeling an aliquot of target protein, and adding a defined amount of the labeled protein to a known amount of unlabeled target protein (“doping” the target molecule population).

However, the present invention is not limited to aspects in which a small percentage of a population of target molecule is labeled. The percentage of a population of a target molecule can be any percentage, from less than 0.1% to greater than 99.9%.

The fluorophore used to label the target protein can be any fluorophore with convenient absorption and emissions spectra for use in the assays. Many fluorophores are known in the art and many are commercially available, for example from Molecular Probes (Eugene, Oreg.). Labeling of target molecule can be direct or indirect. For example, a fluorophore can be chemically coupled to a target molecule using methods known in the art. In the alternative, a fluorophore can be indirectly bound to a target molecule via a specific binding member. A preferred specific binding member for binding a fluorophore to a target molecule is an antibody, such as a monoclonal antibody. The specific binding member can be coupled to a fluorophore, or can optionally bind a fluorophore through a secondary specific binding member, for example through a biotin-streptavidin linkage.

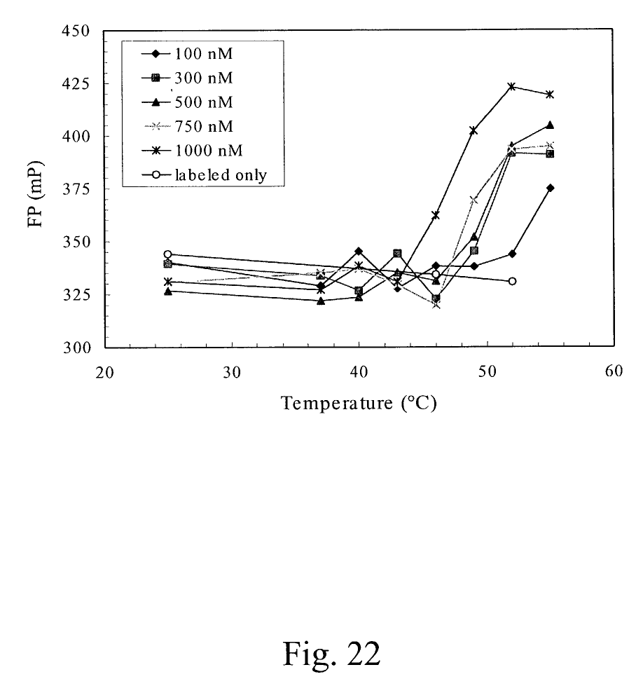

Preferably, a solution comprising a doped population of a target molecule is made up, for example in a buffer, and aliquots of the target molecule solution is added to one or more wells or sample containers. The amount of target molecule used in each sample will vary from target to target. However, the high sensitivity/low background of the assay using FP detection allows for very small amounts of target molecule to be used in these assays, for example, where the target molecule is a protein, from about 0.1 ng to 10 microgram, but preferably the amount of target protein in an assay will be in the range of from about 1 ng to 5 micrograms. The optimal amount of a target protein in an assay sample can be determined empirically by titrating the amount of protein in the assay (see, for example, Example 14 and FIG. 22).

One or more test compounds is added to at least one well or sample container. Test compounds can be made up in solutions comprising buffers, solvents, or other compounds. Test compounds can be added to one or more wells before, after, or at the same time as the target molecule population aliquots are added to one or more wells. Preferably, test compounds are added to at least two wells. It is within the scope of the invention to test several concentrations of the test compound in a given assay. It is also within the scope of the present invention to include more than one test compound in a single test well.

More that one test compound can be added to one or more wells. Preferably, test compounds added to at least two wells are different test compounds, or different amounts or combinations of test compounds. The amount of test compounds introduced into a well can vary, but in many cases will be in the sub-micromolar to micromolar range, such as from about 0.01 micromolar to about 500 micromolar.

Optionally, the target molecule and test compounds (assay mixtures) are incubated for a period of time prior to the heating step. The incubation can be done at any temperature, but, if performed, the pre-incubation is preferably performed at a temperature of not more than 37 degrees C., and more preferably is performed at about 22 degrees C. The pre-heating incubation can be for any length of time, but in cases where it is included, it will typically be for 30 minutes or less.

Preferably, at least one control well comprising the target molecule in the absence of a test compound is included in the assay. Preferably the assay is performed on at least one control well at the same time as the test wells, and all steps of the assay are performed exactly as for the test well or wells; however, it is within the scope of the invention to perform control assays separately, and to record the control data for comparison with test compound assay measurements. One or more measurements from control wells, and values based on measurements from control wells (for example, averages, ratios, anisotropy etc.) whether assayed at the same time as the test wells or not, can be used as a reference value for comparison with one or more test wells.

In the alternative or in addition to including a control well, it is possible to include at least one standard well that comprises a target molecule and at least one compound. The interaction of the compound in the standard well with the target molecule may not be known in advance of the assay, but preferably the degree to which the standard well compound affects denaturation of the target protein is known. In some aspects, standard wells can be test compound wells that are compared with other test compound wells in the assays of the present invention. Preferably, where one or more standard wells is used, the assay is performed on at least one standard well at the same time as the test wells, and all steps of the assay are performed exactly as for the test well or wells; however, it is within the scope of the invention to perform standard assays separately, and to record the standard well data for comparison with test compound assay measurements. One or more measurements from standard wells, and values based on measurements from standard wells (for example, averages, ratios, anisotropy etc.) whether assayed at the same time as the test wells or not, can be used as a reference value for comparison with one or more test wells.

The one or more wells are subjected to conditions at which at least a portion of the target protein is unfolded in the absence of a ligand or test compound. Denaturing conditions can be any conditions that cause loss of secondary, tertiary, or quaternary structure of a target molecule, or alter the three-dimensional conformation of a target molecule, including heat, pH changes, presence of detergents or surfactants, chaotropic agents, salts, chelators, etc. Preferably, the denaturing conditions are elevated temperature and subjecting the test wells to denaturing conditions comprises heating the target molecule and one or more test compounds to one or more predetermined temperatures at which at least a portion of said target molecule is denatured.

In preferred aspects of the present example, the test wells and any control or standard wells will be heated to a single discrete predetermined temperature, termed T_{ATLAS}. T_{ATLAS} can be selected in preliminary experiments in which the target molecule is heated and its degree of unfolding as a function of temperature is monitored (although the identity or any activity of the target molecule need not be known). Preferably, before the assay is performed, the target molecule is characterized to establish a melting (temperature dependent structural unfolding) curve in which a physical measurement that reports on the target molecule's structure is plotted as a function of temperature. The physical measurement can be based on any of a variety of structural determination methods well known in the art, for example, CD, light scattering, UV absorption spectroscopy, differential scanning calorimetry, etc. The melting curve of a target molecule can then used to establish the parameters, including T_{ATLAS} of the assay. Thermal melting can preferably be performed under assay conditions (using buffers, reagents, specific binding members, fluorophores, and FP detection that will be used in test compound assays) to obtain a melting curve under assay conditions (in the absence of test compounds) (see Example 14 and FIG. 22). Preferably, T_{ATLAS} will be selected as a temperature at which assay reagents are stable and the assay has a wide dynamic range and high quality (Z′).

In some cases, it may be desirable to heat the wells to more than one discrete temperature (e.g., T_{ATLAS1}, T_{ATLAS2}, etc.), but this is less preferred. This can be desirable in some cases, for example, if melting curves demonstrate that the target molecule has more than one transition temperature that is indicative of unfolding intermediates. Preferably, however, no more than three discrete temperatures are used in the ATLAS assay, and most preferably the wells are heated to a single T_{ATLAS}.

Heating can be performed in any incubator or sample heating device and is preferably performed using a heating device that allows for rapid, uniform, and accurate heating, and preferably cooling, to precise temperatures, as well as accurate temperature maintenance. For example, many commercially available thermocyclers can be used for this purpose. The assay samples can be held at T_{ATLAS} for any period of time, for example from about 3 minutes to about 6 hours, preferably from about 10 minutes to about one hour. However, the time of T_{ATLAS} incubation is not a limitation of the present invention.

The samples are optionally cooled to a temperature less than T_{ATLAS}. In most cases, assay samples are cooled to approximately room temperature (22 degrees C.). Preferably, where cooling is employed, it is relatively rapid and occurs at a defined rate. In the alternative, it is also possible to maintain the samples at T_{ATLAS} for the detection step. This requires that the fluorescence polarization detection means can interface with a heating element that can maintain the desired temperature during fluorescence polarization detection.

After heating to T_{ATLAS}, and preferably, cooling the samples to a lower temperature fluorescence polarization is detected at one or more wavelengths from one or more test wells and at least one control well. The fluorescence polarization detected in the ATLAS assay provides a measure of the rotational correlation time of the fluorophore. The assay is configured such that denaturation of a target molecule results in changes of the correlation time as aggregates of the target molecule rotate more slowly than non-aggregated targets. Thus the extent of thermal denaturation of the target molecule can be assessed by the FP signal.

Fluorescence polarization detection can be performed by any device that can detect fluorescence polarization at the wavelength emitted by the fluorophore used in the assay. Fluorescence detection devices, including those that detect fluorescence from multiwell plates, are known in the art. The fluorescence detection device can interface with a sample heating device, or can be separate.

In preferred aspects of the present invention, one or more control wells are made up that lack test compound, but that comprise the target molecule and specific binding member(s) in the same amounts as the test wells, and the one or more control wells are heated and analyzed in the same way and at the same time as the test wells. Preferably, one or more control wells are in a multiwell plate that also contains test wells, and the test compound and control assay mixtures are made up at the same time from the same stock concentrations of target molecule, specific binding members, signal molecules, etc.

In the alternative, one or more control wells can be made up at a time other than that when test wells are made up. One or more control wells can be heated and subjected to fluorescence polarization measurements, before or after the test wells are heated. The data from the fluorescence polarization detection of a control well can be recorded and stored, such as in a database.

In some aspects of the present invention, one or more standard wells are provided for comparison with one or more test wells. Standard wells comprise target protein and at least one compound that is either a test compound or a compound whose affect on target unfolding is known. One or more standard wells is also heated and analyzed in the same way and preferably at the same time as the test wells. Where standard wells are used to generate a reference value, they can be one, some, or all of the test wells in one or more assays, and can be used to compute an average value of a detection measurement against which individual test well detection measurements can be compared. Preferably, in aspects of the invention in which standard wells are used, at least one standard well is in a multiwell plate that also contains test wells, and the test compound and standard assay mixtures are made up at the same time from the same stock concentrations of target molecule, specific binding members, signal molecules, etc.

In the alternative, standard wells can be made up at a time other than that when test wells are made up. One or more standard wells can be heated and subjected to fluorescence detection measurements, before or after the test wells are heated. The data from the fluorescence detection of a standard well can be recorded and stored, such as in a database.

Determination of Target Molecule Unfolding

Measurements from one or more test wells are compared with measurements from at least one control well and/or at least one standard well to determine whether any test compounds significantly alter the fluorescence polarization readout. Measurements from one or more control wells or one or more standard wells, or values derived therefrom, used for comparison with test well measurements or values, are herein referred to as reference values. Test wells that differ from control wells by more than a particular amount or percentage in fluorescence polarization, can be identified as wells in which the target molecules has unfolded to a significantly different degree than in control wells lacking test compound. Test wells that differ from standard wells by more than a particular amount or percentage in fluorescence polarization, can be identified as wells in which the target molecule has unfolded to a significantly different degree than in standard wells comprising one or more different compounds.

In most (but not all) cases, the difference in fluorescence signal or signals or determinations based on fluorescence signals will indicate that the test compound has to some degree protected the target molecule from unfolding in response to elevated temperature. When target molecules unfold and aggregate, the fluorescence polarization signal increases due to the longer rotational correlation of the aggregated versus non-aggregated target that comprises a fluorophore. However, it is also possible to identify compounds that promote unfolding of the target under denaturing conditions by detecting a decrease in the fluorescence polarization signal with respect to controls. Compounds that promote unfolding of the target can also be ligands of the target. Without being bound to any particular mechanism, in some cases compound binding may make a target more susceptible to unfolding at a particular temperature.

Identification of Ligands

Test compound wells that differ from control wells by more than a particular amount or percentage in fluorescence polarization can be identified as first screen hits. Those skilled in the art can determine reasonable criteria for identifying first screen hit, such as, for example 20% or greater difference from control data, or preferably a 50% or greater difference from control data.

Preferably, first screen hits are rescreened in the same assay format in which they were originally identified. First screen hits that differ from control wells by more than a particular amount or percentage in fluorescence polarization in a second assay are called duplicate hits.

Duplicate hits can be subjected to a titration series in which they assayed at a range of concentrations (see Example 17). Duplicate hits that are titratable, that is, that show concentration dependency in the assay, are potential ligands for the target molecule. IC 50 values can be determined from these assays.

Test compounds identified as potential target molecule ligands can be tested in other types of assays for independent confirmation of target molecule binding. Examples of such assays are ELISA, filter binding, isothermal calorimetry, or other binding assays as they are known in the art.

High Throughput Screening

The present invention is particularly well-suited to high throughput screening, in which a multiplicity of test compounds can be tested at the same time. Because of the high degree of sensitiviy and low background of fluorescence polarization detection, small amounts of protein and correspondingly small volumes can be used for assays. In high throughput assays, samples are preferably made up in wells of multiwell plates. However, other sample containers can be used. For example, the sample containers can be indentations of a surface, or can be capillaries or tubes for holding small volume (sub-milliliter) liquid samples. Preferably, the assay is formatted for high throughput or ultra high throughput screening (HTS or UHTS) involving a multiplicity, and preferably hundreds, of samples, and thus the assays are most conveniently performed in wells of for example, 96, 384,1536, or 3456 well plates. Plate heating and plate fluorescence detection systems as they are known in the art or designed for the methods of the present invention can be used.

The ATLAS assay can easily be configured such that a minimum of pipeting steps are required. For example, in Example 16, two reagent mixes are used: one containing test compound, and one containing labeled target protein. Preferably, liquid handling devices are used for dispensing sample components. In addition, the assay can be performed within a short time period, as assay samples can be assembled, rapidly heated to a single temperature, incubated for less than an hour, rapidly cooled, and detected.

The addition of reagents, as well as heating, incubations, cooling and detection steps can be automated. In a preferred aspect of the present invention, an integrated system employs robotics to dispense reagents, and to move plates comprising test wells to and from dispensing areas, heating/cooling devices, and fluorescence plate readers. Preferably the integrated system is computerized and programmable, and contains software for sample analysis

V. Compounds Identified Using the Methods of the Present Invention

The present invention also includes compounds identified using the methods of the present invention as ligands of target molecules. Such compounds are useful as pharmacological compounds and as starting points for medicinal chemical studies to identify derivatives or modifications of identified compounds. Such medicinal chemical studies can further screen compounds and derivatives thereof for activities, pharmacology, toxicology and the like as described herein and as is known the art.

Pharmacology and Toxicity of Test Compounds

Based on such nexuses, appropriate confirmatory in vitro and in vivo tests of pharmacological activity, and toxicology, and be selected and performed. The methods described herein can also be used to assess pharmacological selectivity and specificity, and toxicity. Identified test compounds can be evaluated for toxicological effects using known methods (see, Lu, Basic Toxicology, Fundamentals, Target Organs, and Risk Assessment, Hemisphere Publishing Corp., The structure of a test compound can be determined or confirmed by methods known in the art, such as mass spectroscopy. For test compounds stored for extended periods of time under a variety of conditions, the structure, activity and potency thereof can be confirmed. Identified test compounds can be evaluated for a particular activity using are-recognized methods and those disclosed herein. For example, if an identified test compound is found to have anticancer cell activity in vitro, then the test compound would have presumptive pharmacological properties as a chemotherapeutic to treat cancer. Such nexuses are known in the art for several disease states, and more are expected to be discovered over time. Washington (1985); U.S. Pat. No; 5,196,313 to Culbreth (issued Mar. 23, 1993) and U.S. Pat. No. 5,567,952 to Benet (issued Oct. 22, 1996)). For example, toxicology of a test compound can be established by determining in vitro toxicity towards a cell line, such as a mammalian, for example human, cell line. Test compounds can be treated with, for example, tissue extracts, such as preparations of liver, such as microsomal preparations, to determine increased or decreased toxicological properties of the test compound after being metabolized by a whole organism. The results of these types of studies are predictive of toxicological properties of a chemical in animals, such as mammals, including humans. Alternatively, or in addition to these in vitro studies, the toxicological properties of a test compound in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the test compound, the skilled artisan would not be burdened to determine appropriate doses, LD₅₀ values, routes of administration and regimes that would be appropriate to determine the toxicological properties of the test compound. In addition to animal models, human clinical trials can be performed following established procedures, such as those set forth by the United States Food and Drug Administration (USFDA) or equivalents of other governments. These toxicity studies provide the basis for determining the efficacy of a test compound in vivo.

Efficacy of Test Compounds

Efficacy of a test compound can be established using several art recognized methods, such as in vitro methods, animal models or human clinical trials (see, Creasey, supra (1979)). Recognized in vitro models exist for several diseases or conditions. For example, the ability of a test compound to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic, disease or condition, an acceptable in vitro or animal model is available. The skilled artisan is armed with a wide variety of such models as they are available in the literature or from the USFDA or the National Institutes of Health (NIH). In addition, these in vitro methods can use tissue extracts, such as preparations of liver, such as microsomal preparations, to provide a reliable indication of the effects of metabolism on a test compound. Similarly, acceptable animal models can be used to establish efficacy of test compounds to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model which confirms the validity of this model (see, McDonough, Phys. Ther. 62:835-839 (1982)). When choosing an appropriate model to determine efficacy of test compounds, the skilled artisan can be guided by the state of the art, the USFDA or the NIH to choose an appropriate model, doses and route of administration, regime and endpoint and as such would not be unduly burdened.

In addition to animal models, human clinical trials can be used to determine the efficacy of test compounds. The USFDA, or equivalent governmental agencies, have established procedures for such studies.

Selectivity of Test Compounds

The in vitro and in vivo methods described above also establish the selectivity of a candidate modulator. It is recognized that chemicals can modulate a wide variety of biological processes or be selective. Panels of cells as they are known in the art can be used to determine the specificity of the a test compound (WO 98/13353 to Whitney et al., published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a test compound can be established in vitro by testing the toxicity and effect of a test compound on a plurality of cell lines that exhibit a variety of cellular pathways and sensitivities. The data obtained form these in vitro toxicity studies can be extended to animal model studies, including human clinical trials, to determine toxicity, efficacy and selectivity of a test compound.

The selectivity, specificity and toxicology, as well as the general pharmacology, of a test compound can be often improved by generating additional test compounds based on the structure/property relationship of a test compound originally identified as having activity. There may also be a structural/property relationship of a set of test compounds that display varying degrees of activity. Test compounds can be modified to improve various properties, such as affinity, life-time in blood, toxicology, specificity and membrane permeability. Such refined test compounds can be subjected to additional assays as they are known in the art or described herein. Methods for generating and analyzing such compounds or compositions are known in the art, such as U.S. Pat. No. 5,574,656 to Agrafiotis et al.

Pharmaceutical Compositions

The present invention also encompasses a compound identified using the methods of the present invention, or a portion or derivative thereof, in a pharmaceutical composition comprising a pharmaceutically acceptable carrier prepared for storage and preferably subsequent administration, which have a pharmaceutically effective amount of the peptide or protein in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The compound of the present invention can be formulated and used in tablets, capsules or elixirs for oral administration; suppositories for rectal administration; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as liposomes, can be used.

The pharmaceutically effective amount of a compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperitoneally, employing a variety of dosage forms. Such methods can also be used in testing the activity of a compound of the present invention in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the compound.

In non-human animal studies, applications of the pharmaceutical compositions are commenced at higher dose levels, with the dosage being decreased until the desired effect is no longer achieved or adverse side effects are reduced of disappear. The dosage for the compounds of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ disfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, nasal, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Intracellular delivery of drugs may be acheived by linking peptides such as the translocating domain of the tat protein of HIV to the agent. Linkage of hydrophobic molecules such as biotin to the attached tat peptide or similar translocating peptides may improve intracellular delivery further (Chen et al. Analyt. Biochem. 227: 168-175 (1995)). Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The compounds of the present invention, and pharmaceutical compositions that include such compounds, can be used to treat a variety of ailments in a patient, including a human. The compounds of the present invention can have antibacterial, antimicrobial, antiviral, anticancer cell, antitumor and cytotoxic activity. A patient in need of such treatment can be provided a compound of the present invention, or a portion thereof, preferably in a pharmacological composition. The amount, dosage, route of administration, regime and endpoint can all be determined using the procedures described herein or by appropriate government agencies, such as the United Stated Food and Drug Administration.

### EXAMPLES

### Example 1

### Characterization of the X90 Target Protein

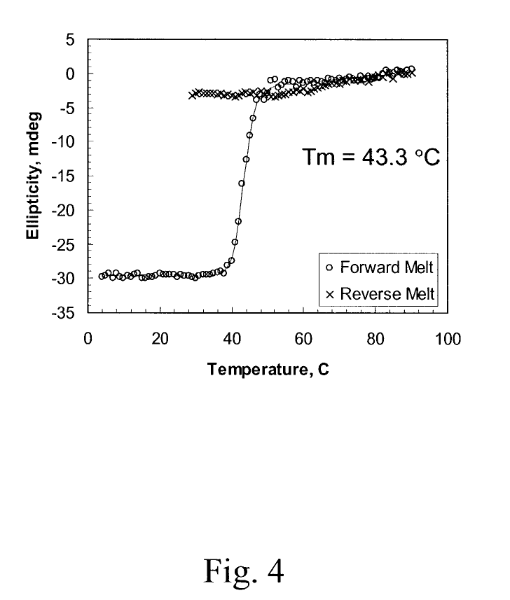

X90 was selected as a target protein. Recombinant X90 was produced in E. coli and purified using standard methods. Thermal melting of X90 was performed by heating of 40 micrograms of the protein in a volume of 0.2 ml of ATLAS buffer (50 mM Tris, pH 7.5; 250 mM NaCl; 0.1% Tween 20; 0.5 mM DTT; and 0.5% NaN₃) and heating at a rate of 1 degree C. per minute in a Circular Dichroism Spectrophotometer, Model 62ADS, made by AVIV (Lakewood, N.J.) up to greater than our about 90 degrees C. followed by cooling. Ellipticity was plotted as a function of temperature, shown in FIG. 4. The Tm was calculated to be 43.3 degrees C.

### Example 2

### X90 Target Protein Assay Development

The assay for compounds that bind X90 was developed using a monoclonal antibody specific for the unfolded form of X90. To make the antibody, recombinant X90 protein was first denatured in SDS. The X90 protein was run on a SDS-PAGE gel. The gel was stained with Coomassie Blue, and the stained band was cut out of the gel and the protein electroeluted out of the gel slice. Mice were injected with the electroeluted denatured protein. Monoclonals were screened for the ability to recognize unfolded X90 protein, but not folded X90 protein. Monoclonal antibodies were developed, screened, and purified using methods known in the art.

The monoclonal antibody was biotinylated by making a stock of 0.5 ml of 2.0 mg/ml in 50 mM bicarbonate buffer, pH 7.8. 37.5 microliters of 1 mg/ml NHS-LC-biotin was added, and the mixture was incubated on ice for 2 hours. Following incubation, the biotinylated antibody was purified from free biotin using dialysis, gel filtration, and in some cases washing with buffers through filters with appropriate molecular weight cut-offs (e.g. Centriprep YM-50). The degree of biotinylation of the monoclonal antibody was determined using the HABA/Avidin system from Pierce Chemical Co. (Pierce document #0212, Pierce ImmunoPure HABA Cat #28010, Pierce ImmunoPure D-Biotin Cat. #29129, Pierce ImmunoPure Avidin Cat. #21121).

To configure the assay, thermal melting curves were generated using assay reagents and varying concentrations of protein (FIG. 5). Stock solutions of biotinylated antibody (200 ng/microliter) and target protein X90 (50 ng/microliter) were made in ATLAS Buffer (50 mM Tris, pH 7.5; 250 mM NaCl; 0.10% Tween 20; 0.5 mM DTT; and 0.05% NaN₃). An ATLAS Assay Mix consisting of 1 ng/microliter of biotinylated antibody, and 1% DMSO, and varying concentrations of X90 target protein in ATLAS Buffer was also made up. Six different assay mixtures containing final concentrations of 20 ng of biotinylated antibody, 1% DMSO, and either 0, 3, 6, 12, 24, or 48 ng of X90 target protein, were made up in ATLAS Buffer. Each assay mixture was used for 3 sample wells of 384 well assay plates, each containing 20 microliters of sample. The assay plates were placed in MWG PrimusHT Thermocyclers, the lids were closed, and the lids were heated to 70 degrees C. The thermocyclers were programmed to heat to incubation temperature ranging from 35 to 60 degrees C. at a rate of 2.0 degrees C. per second, and then to hold temperature at the incubation temperature for 30 min 0 sec. The temperature then decreased to 22.0 degrees C. at a rate of 2.0 degrees C. per second. The cycler lids were then opened and 10 microliters of Revelation Mix (300 mM KF; 1.8 ng/microliter anti-6HIS Ab labeled with Europium Cryptate; and 5 ng per microliter streptavidin labeled with XL665) were added to each sample well of each of the 384 well assay plates. The plates were then incubated 40 min at room termperature, and the plates were read in a Victor V in LANCE mode at 665 nm and 620 nm.

The ratio of 665 nm/620 nm fluorescence was calculated and plotted as a function of incubation temperature. FIG. 5 shows the results of the average of two of these experiments, confirming that the signal increases with temperature and, by comparison with the thermal unfolding CD spectra, with the unfolding of the target protein. (The signal does not increase in the absence of target protein.) At high temperatures, the assay signal starts to decrease from its maximum. This decrease is presumably due to antibody melting at higher temperatures. The temperature profile is shown in FIG. 6 with a calculated Tm of 47.3° C. This configured assay runs in 384 well plates (20 microliters/well).

### Example 3

### X90 Target Protein Assay Validation

Stock solutions of biotinylated antibody (200 ng/microliter) and target protein X90 (50 ng/microliter) were made in ATLAS Buffer (50 mM Tris, pH 7.5; 250 mM NaCl; 0.10% Tween 20; 0.5 mM DTT; and 0.05% NaN₃). An ATLAS Assay Mix consisting of 1 ng/microliter of biotinylated antibody, 0.6 ng/microliter of target X90 protein (12 ng/well), and 1% DMSO in ATLAS Buffer was also made up. Twenty microliters of ATLAS Assay Mix was added to each of 384 wells of ten 384 well PCR assay plates (Nalge Nunc International, Cat. #264582), and to each of 384 wells of 10 identical 384 well plates to be used as controls. The control plates were incubated at 4 degrees C. for 30 minutes. The assay plates were placed in MWG PrimusHT Thermocyclers, the lids were closed, and the lids were heated to 70 degrees C. The thermocyclers were programmed to heat to 53.0 degrees C. at a rate of 2.0 degrees C. per second, and then to hold temperature at 53.0 degrees C. for 30 min 0 sec. The temperature then decreased to 22.0 degrees C. at a rate of 2.0 degrees C. per second. The cycler lids were then opened and 10 microliters of Revelation Mix (300 mM KF; 1.8 ng/microliter anti-6HIS Ab labeled with Europium Cryptate; and 5 ng per microliter streptavidin labeled with XL665) were added to each sample well of each of the 384 well assay plates. The plates were then incubated 40 min at room temperature, and the plates were read in a Victor V in LANCE mode at 665 nm and 620 nm.

TABLE 1
Properties for LANCE Measurements on the Victor V
Fluorescence Reader.

| Properties for LANCE | | |
|---|---|---|
| Measurements on the Victor V | LANCE 620 nm | LANCE 665 nm |
| Flash Energy Area | High | High |
| Flash Energy Level | 199 | 199 |
| Excitation Filter | D320 | D320 |
| Light Int. Cap. | 1 | 1 |
| Light Int. Ref. Level | 19 | 19 |
| Emission Filter | D620 Slot A6 | D665 Slot A7 |
| Emission Aperture | Normal | Normal |
| Counting Delay | 1:50 2:0 | 1:50 2:0 |
| Counting Window | 1:400 2:0 | 1:400 2:0 |
| Counting Cycle | 1000 | 1000 |
| Flash Abs | No | No |
| Beam Size | Normal | Normal |
| Second Measurement | Not Checked | Not Checked |

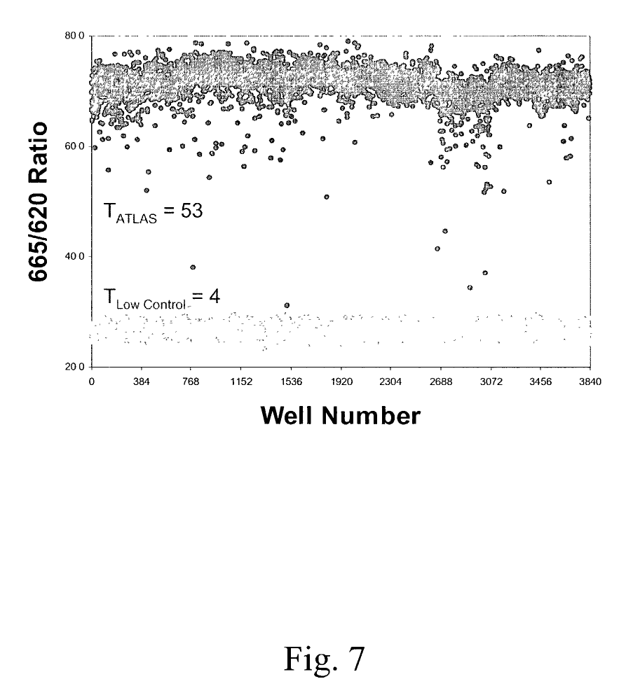

The result of the assay is shown in the graph of FIG. 7. The Z′ value was calculated in order to assess assay robustness. The statistics and the corresponding Z′ value are given in the table 1. A Z′ value of 0.72 for the configured target X90 was calculated using the formula: Z′=1−(3*SD_{—}53C+3*SD_{—}4C)/(Ave_{—}53C−Ave_{—}4C), where SD_{—}53C, SD_{—}4C, Ave_{—}53C and Ave_{—}4C are the standard deviations and average at the two temperatures (53° C. and 4° C.). This value of 0.72 falls well within the acceptable range of 0.50 to 1.00, indicating a robust assay.

TABLE 2
Assay validation statistics.
Assay Z′ Value: 0.72

| | 4° C. | 53° C. |
|---|---|---|
| | Plates | Plates |
| Average | 26.6 | 71.2 |
| SD | 0.9 | 3.3 |
| CV (%) | 3.5 | 4.6 |
| n | 3,840 | 3,840 |

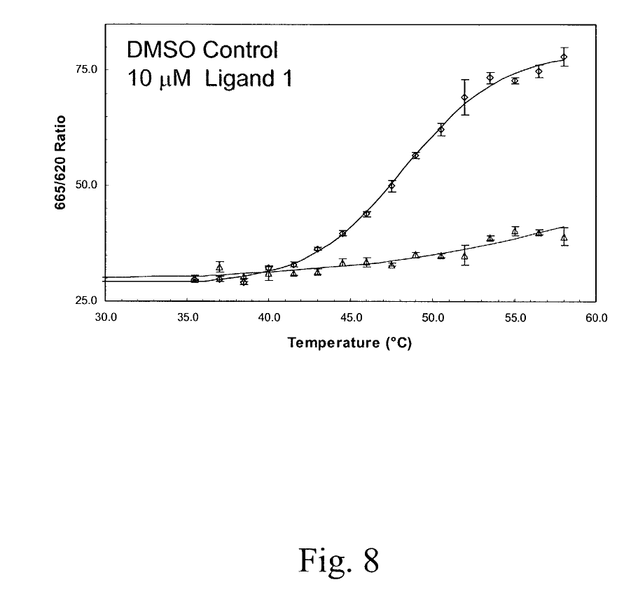

As a further test of our screening method, a thermal melting was performed in the TR-FRET assay format in the presence of a known ligand to target X90 (FIG. 8). A known ligand to target X90 was added to a final concentration of 10 micromolar to the ATLAS Assay Mix described above. For each temperature, the assay performed as described above. Briefly, 3 sample wells of a 384 well plate were loaded with 20 microliters of the assay mix, and the plates were heated to 53 degrees C. After cooling to 22 degrees C., ten microliters of Revelation Mix were added to each sample well, the plates were incubated at room temperature and then read in the Victor V plate readers. DMSO was used in control wells to control for the DMSO present in the ligand stock solution. In the presence of the ligand, the melting transition is pushed to higher temperature, indicating the ligand has conferred thermal protection to the target protein.

### Example 4

### High Throughput Screen for Ligands of X90

Two sets of twenty-two 384 well plates were screened using the ATLAS Mix and Revelation Mix described above. T_{ATLAS} was 53 degrees C. For each 384 well plate, 32 wells served as assay controls (each having 2 microliters of 10% DMSO per well in place of compound), while the remaining 352 wells contained the compound (2 microliters at a concentration of 100 micromolar in 10% DMSO) for screening. Each set of twenty-two plates containing 352 compounds each (22×352=7744 compounds); the final compound concentration in the assay was 10 micromolar.

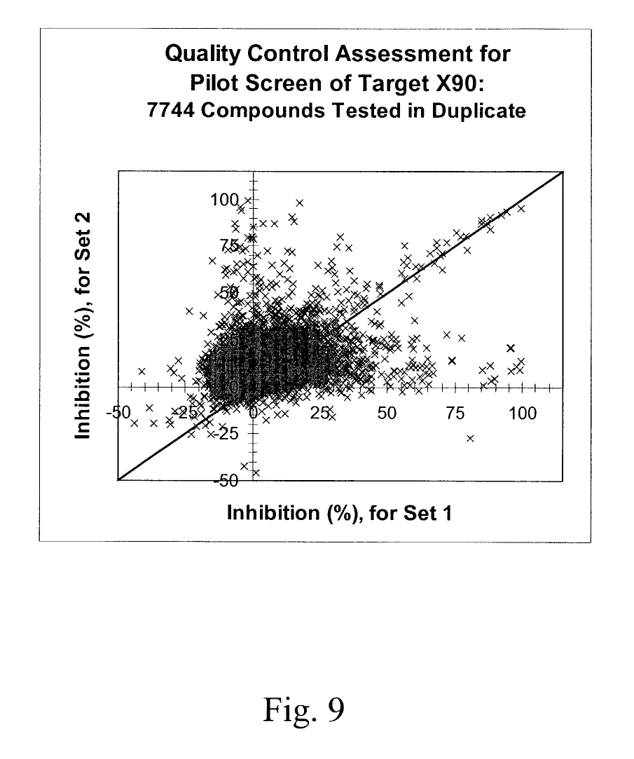

To assess the quality control of the assay's response when screening compounds, 7,744 compounds were screened twice for target X90. The degree of assay inhibition for each compound is plotted; the results from the two screens are plotted against each other (FIG. 9). The black diagonal line represents the ideal case where the compounds show exactly the same degree of inhibition in both screens. Compounds that showed a significant difference from controls in both of the two screens were considered duplicate hits. A number of such duplicate hits were obtained from the screen of 7,744 compounds.

### Example 5

### Titration of Duplicate X90 Target Protein Assay Hits

Concentrations of compounds that were identified as duplicate hits were titrated by performing a series of 2-fold serial dilutions, creating a series of 11 concentrations for each compound; the highest concentration was 100 micromolar. These compounds were assayed using the ATLAS Mix, Revelation Mix, and assay protocol described above; T_{ATLAS} was 53 degrees C. The titration curves of twenty of these hits are shown in FIG. 10.

### Example 6

### Independent Validation of X90 Target Binding by Duplicate Hit Compounds

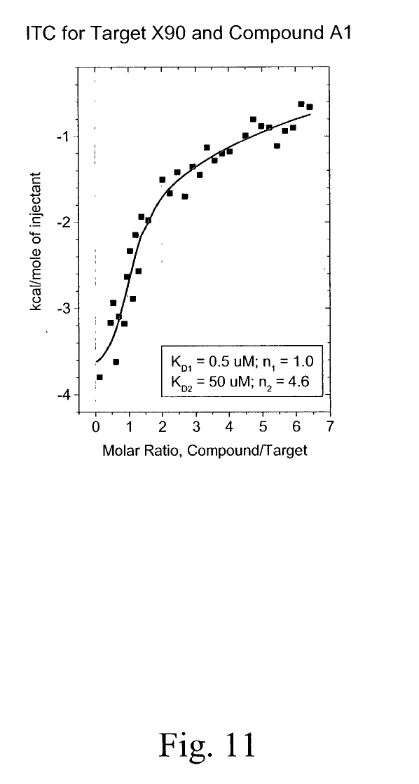

To obtain independent validation of compound binding, some of the titratable duplicate hits was subjected to IsoThermal Calorimetry (ITC); the results from one of these compounds is shown in FIG. 11. ITC measurements were performed on a Microcal VP-ITC microcalorimeter (MicroCal Inc., Northampton, Mass.). Samples were filtered and degassed for ten minutes prior to loading. Experiments were performed with a sample temperature of 25 degrees C. The buffer was 50 mM Tris, 250 mM NaCl, 1 mM TCEP, 1% DMSO.

The concentration of X90 in the sample cell was 8 micromolar. The titration was performed by controlled injections of 230 micromolar compound into the sample cell, allowing 400 seconds between injections. The peaks produced over the course of the titration were integrated and used to obtain a plot of the enthalpy change versus the molar ratio of species in the cell. A control experiment was performed to determine the contribution to the binding enthalpy from the heat of dilution of the compound into the buffer. The net enthalpy for the interaction between compound and protein was determined by subtraction of the heat of dilution component. Curve fitting was performed using the ORIGIN software to determine the dissociation constants and the number of binding sites for the interaction between compound and protein. The data indicated there is one tight binding site for the compound with a submicromolar K_{D} (which agrees well with the IC50 value from the titration experiment). There is also a set of much weaker binding sites for the compound: an average of 4.6 compounds per target bind with an effective K_{D} that is higher by about two orders of magnitude.

### Example 7

### Characterization of the DB7 Target Protein

DB7 was selected as a target protein. Recombinant DB7 having a 6xHis tag inserted at the N-terminus through genetic engineering was produced in E. coli and purified using standard methods. The following biophysical characterizations were performed using the DB7 protein having the 6xHis insertion.

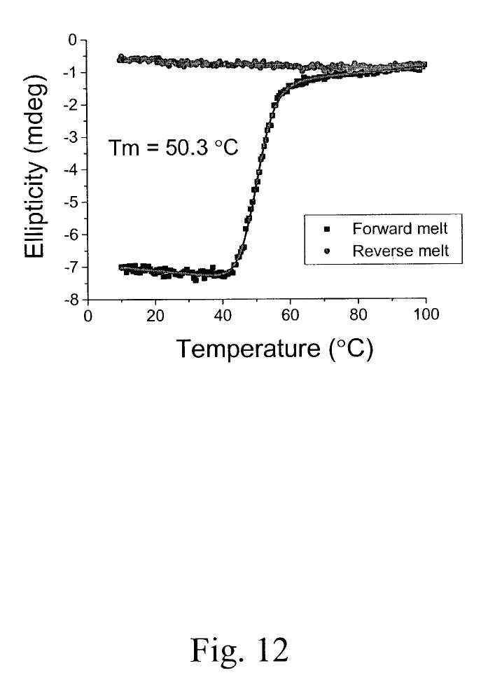

Thermal melting of DB7 was performed by heating 0.2 mg per ml of the protein in a volume of 0.2 ml and heating at a rate of one degree C. per min up to greater than or about 90 degrees C., followed by cooling, in a Circular Dichroism Spectrophotometer, Model 62 ADS (AVIV, Lakewood, N.J.). Ellipticity was measured and plotted as a function of temperature, shown in FIG. 12. The CD thermal melting profile shows that the protein undergoes irreversible unfolding as the temperature is increased; the midpoint temperature of the unfolding transition is 50.3° C.

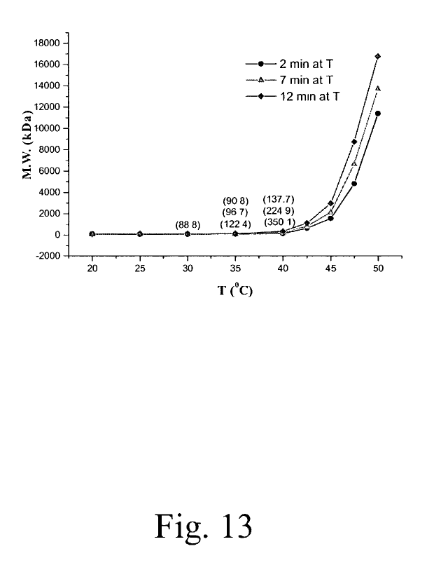

The protein was also analyzed by light scattering to assess DB7 aggregation upon unfolding by molecular weight (FIG. 13). The increase in apparent molecular weight at higher temperatures indicates the unfolded target protein aggregates once it unfolds.

### Example 8

### DB7 Target Protein Assay Development

For this assay we used generic reagents instead of antibodies raised against the denatured form of the target protein. Unlike the assay format detailed in Examples 2-5, this assay used time resolved fluorescence for detection of aggregates. This allowed us to detect aggregation of the target protein upon unfolding via energy transfer from donor to acceptor. In addition, a higher concentration of protein was used in the assay when compared with the assay illustrated in Example 3, as this was found to promote the formation of aggregates at the screening temperature, or T_{ATLAS}.

Two approaches were taken in developing TR-FRET assays. The approaches differed in the method of attaching fluorophores to the target protein. In both cases, attachment of fluorophores was through binding of antibodies to an attached tag (6xHis) of the DB7 protein.

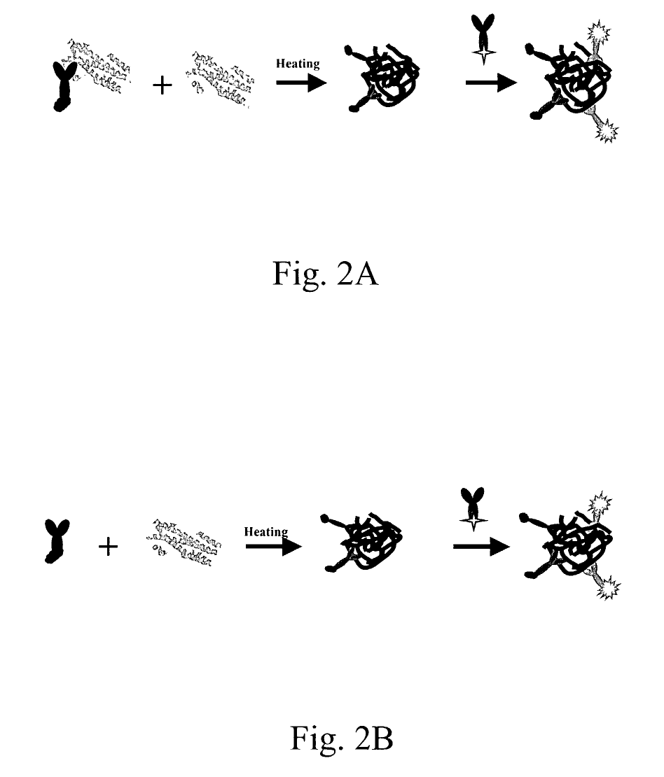

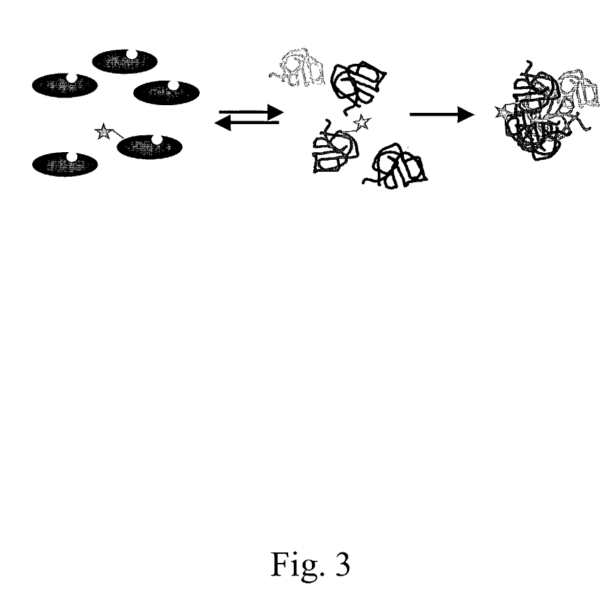

The first TR-FRET assay configuration involved pre-binding half of the DB7 protein to be used in the assay with an anti-6xHis antibody labeled with the donor fluorophore, adding the non-antibody-bound half of protein, heating the mixture, and then adding an anti-6xHis antibody labeled with the acceptor fluorophore as a revelation step prior to reading (FIG. 2a). The second approach involved mixing of all the DB7 protein to be used in the assay with the anti-6xHis antibody labeled with the donor fluorophore, heating the mix, and then adding an anti-6xHis antibody labeled with the acceptor fluorophore as a revelation step prior to reading (FIG. 2b).

The detection format for the DB7 assay was based on Time Resolved Energy Transfer Fluorescence (TR-FRET or “TRET”), and utilized homogenous time resolved fluorescence (HTRF) reagents commercially available through Packard Biosciences. The Europium Cryptate moiety (Donor Fluorophore) was attached to an anti-6xHis tag monoclonal antibody and the XL665 moiety (Acceptor Fluorophore) was attached to an anti-6xHis tag monoclonal antibody (FIG. 2). In both approaches, the aggregation that results from heating of the protein allows a sandwich to be formed between the donor fluorophore, aggregated DB7, and the acceptor fluorophore. The DB7 aggregate is detected through energy transfer from donor to acceptor, now in close proximity as shown in FIG. 2. The abundance of aggregates in solution upon heating, and therefore the amount of time-resolved emission from the acceptor fluorophore, will be proportional to the amount of aggregated DB7 protein in solution. Based on this, if a compound binds to the DB7 protein, it will shift the aggregated to folded ratio, thus altering the amount of the observed energy transfer between donor and acceptor.

Configuration A (Pre-labeling of Half of the Target Protein)

In Configuration (A), half of the DB7 protein was pre-coupled to the anti-6xHis antibody labeled with Europium Cryptate in a 10 microliter volume, prior to adding the other half of DB7 protein in 5 microliter volume and heating the mixture. After cooling the sample mixtures, the anti-6xHis antibody labeled with XL665 acceptor component was added in a 5 microliter volume and the signals are read at 620 nm for europium and 665 nm for XL665.

Performing measurement at two wavelengths (620 nm & 665 nm) allowed a ratio of 665/620 to be calculated as Ratio=(signal665/signal620)×1000 and reported as the 665/620 ratio, thus eliminating nearly all compound interference, especially since the measurement was delayed for 50 micro seconds after excitation and prior to sensing for 400 micro seconds (see, Table 1).

The assay was performed at a series of temperatures using several protein concentrations and detection was through time-resolved fluorescence resonance energy transfer (TR-FRET). Two mixtures were made, a DB7+anti-His Ab (Eur. Crpt.) mix and a DB7 mix. The DB7+anti-His Ab (Eur. Crpt.) mix contained 50 mM sodium phosphate pH 6.2, 200 mM NaCl, 0.10% Tween 20, 1 mM DTT, a variable amount of DB7 protein, and 1.2 microgram per milliliter of Anti-6His Ab labeled with Europium Cryptate. The concentration of DB7 protein in the DB7+anti-His Ab (Eur. Crpt.) mix varied from 0 to 8.8 nanograms per microliter, to provide from 0 to 88 ng of antibody labeled DB7 protein per well. The DB7 mix contained 50 mM sodium phosphate pH 6.2, 200 mM NaCl, 0.10% Tween 20, 1 mM DTT, and from 0 to 17.6 nanograms per microliter of DB7 protein, to provide from 0 to 88 ng of unlabeled DB7 protein per well. In performing the assays, equal amounts of labeled and unlabeled protein were added, so that each mix always contributed 50% of the total DB7 protein in the assay, and the total amount of DB7 protein in the wells varied from 0 to 176 ng.

Two microliters of DMSO was added to each of 3 wells of 384-well plates to mimic the DMSO present during compound screening. Five microliters of DB7 mix and ten microliters of DB7+anti-His Ab (Eur. Crypt.) mix were then added to each well. The plates were then incubated at various temperatures, ranging from 30 degrees C. to 62 degrees C. at two degree increments, for 30 minutes. Five microliters of revelation mix containing 50 mM sodium phosphate pH 6.2, 200 mM NaCl, 0.10% Tween 20, 1 mM DTT, and 200 ng per 5 microliters of anti-6xHis AB labeled with XL665 were then added to the sample wells. The plates were then incubated for 30 minutes at room temperature. Fluorescence from the wells were read in a Victor V (PerkinElmer) equipped with emission filters at 620 and 665 nm in LANCE mode at both 620 and 665 nm.

As can be seen in FIG. 14A, which shows thermal melting as a function of incubation temperature for a range of DB7 concentrations, 22, 44, and 88 ng of protein (in a 17 microliter reaction volume) give strong 665 nm/620 nm signals. The assay signal increased with increasing DB7 protein concentration and approached saturation for the higher protein concentrations (FIG. 14a). By curve-fitting this data, midpoint transition temperatures of 47.5 and 47.0 degrees C. for 44 ng and 88 ng, respectively were obtained (FIG. 15).

Configuration B (Pre-labeling of Essentially All of the Target Protein)

Similarly, in Configuration (B), all of the DB7 protein was heated in the presence of the anti-6xHis antibody labeled with europium cryptate in 15 microliter volume. After cooling, the anti-6xHis antibody labeled with XL665 acceptor component was added in a 5 microliter volume and signals were read in the same manner as above. As in Configuration (A), the abundance of aggregates in solution upon heating, and therefore the amount of time-resolved emission from the acceptor fluorophore, will be proportional to the amount of aggregated DB7 protein in solution. Based on this, if a compound binds to the DB7 protein, it shifts the aggregated to folded ratio, thus altering or reducing the amount of the observed energy transfer between donor and acceptor.

The assay was performed at a series of temperatures using several protein concentrations and detection was through time-resolved fluorescence resonance energy transfer (TR-FRET).

A DB7+anti-His Ab (Eur. Crypt.) mix was made up that contained 50 mM sodium phosphate pH 6.2, 200 mM NaCl, 0.10% Tween 20, 1 mM DTT, a variable amount of DB7 protein, and 2 micrograms per milliliter of Anti-6xHis Ab labeled with Europium cryptate. The amount of DB7 protein in the DB7+anti-His Ab (Eur. Crypt.) mix varied from 0 to 2.9 nanograms per microliter, to give from 0 to 44 nanograms of protein per well.

Two microliters of DMSO was added to each of 3 wells of 384-well plates to mimic the DMSO present during compound screening. Fifteen microliters of DB7+anti-His Ab (XL665) mix were then added to each well. The plates were then incubated at various temperatures, ranging from 30 degrees C. to 62 degrees C. at two degree increments, for 30 minutes. Five microliters of revelation mix containing 50 mM sodium phosphate pH 6.2, 200 mM NaCl, 0.10% Tween 20, 1 mM DTT, and 200 ng per 5 microliters of anti-6xHis Ab labeled with XL665 were then added to the sample wells. The plates were then incubated for 30 minutes at room temperature. Fluorescence from the wells was read in a Victor V (PerkinElmer) equipped with emission filters at 620 and 665 nm in LANCE mode at both 620 and 665 nm.

As can be seen in FIG. 14B, which shows thermal melting as a function of incubation temperature for a range of DB7 concentrations, 44 ng of protein (in a 17 microliter reaction volume) gave strong 665 nm/620 nm signals.

Thus, the ATLAS assay for the DB7 target protein has been configured using TR-FRET as a detection method together with commercially available FRET reagents. The TR-FRET assay has a Tm of 47.5° C. and the biophysics data showed a Tm of 50.3° C. The antibody concentrations having both donor and acceptor attached fluorophores were held constant in these experiments. In this assay system, detection required a higher concentration of protein than in the assay system of Example 1, where 3 ng of protein in a 50 microliter assay volume could adequately report on protein unfolding, supporting the concept that protein aggregation is indeed measured by the TR-FRET detection system in this assay. Thus, the obtained data for both configurations show dependency of aggregation on DB7 protein concentration, while the “no protein” control signal did not increase during the course of the assays.

### Example 9

### DB7 Target Protein Assay Validation

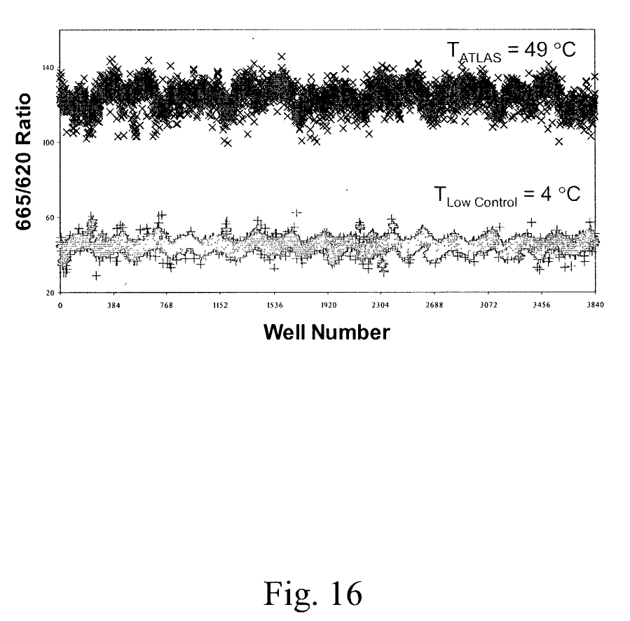

Validation of the Configuration (A) assay involved running ten 384 well plates at the determined T_{ATLAS} (49° C.) and ten plates at low temperature (4° C.), using 44 ng DB7 protein per 15 μL well volume (FIG. 16). The assay robustness has been measured by running ten plates at both the screening temperature T_{ATLAS} (49° C.) and a control temperature (4° C.). The Z′ value for this validation was calculated to be 0.63, well within the acceptable range of 0.5 to 1.0.

TABLE 3
Assay validation statistics.
Assay Z′ Value: 0.63

| | 4° C. | 49° C. |
|---|---|---|
| | Plates | Plates |
| Average | 45 | 124 |
| SD | 3.3 | 6.5 |
| CV (%) | 7.5 | 5.3 |
| n | 3,840 | 3,840 |

### Example 10

### High Throughput Screen for Ligands of Target Protein DB7

7,744 compounds were tested in duplicate with target protein DB7. Two sets of twenty two 384 well plates were screened using Configuration A described above, with T_{ATLAS}=49° C. For each 384 well plate, 32 wells served as assay controls (1.5 ul of 10% DMSO per well), while the remaining 352 wells contained the test compounds (2 ul at 100 micromolar in 10% DMSO) for screening. Each set of twenty-two plates containing 352 compounds each (22×352=7744 compounds); final compound concentration in the assay was 10 micromolar.

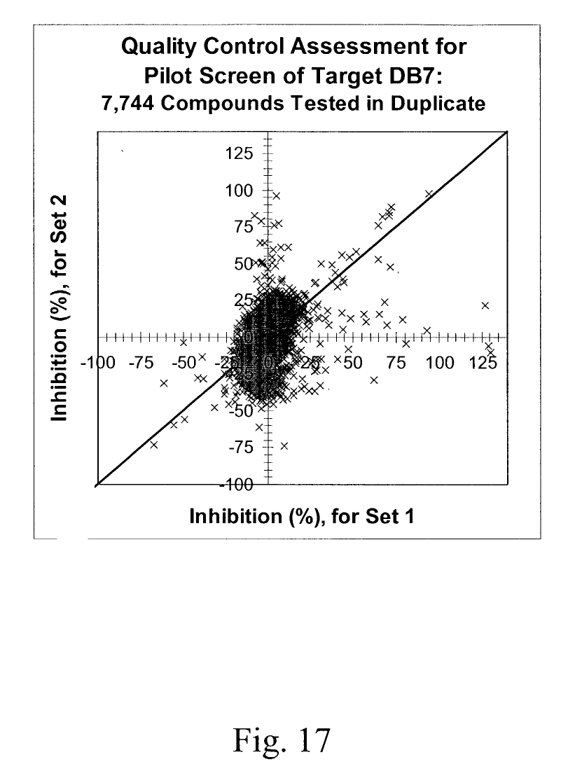

Quality Control assessment of wells containing test compounds from the duplicate screens is shown in the scatter plot of FIG. 17.

### Example 11

### Titration of DB7 Target Protein Assay Duplicate Hits

Compounds that showed assay inhibition at the screening concentration of 10 micromolar in two screens were tested over a range of concentrations to test concentration dependent assay inhibition and to calculate the IC50 for each compound. Compound concentrations were titrated by performing a series of 2-fold serial dilutions, creating a series of 11 concentrations for each compound; the highest concentration was 100 micromolar. These compounds were assayed using Configuration (A) described above; T_{ATLAS}=49° C. The results of three such titrations are shown in FIG. 18.

### Example 12

### Independent Validation of DB7 Target Binding by Duplicate Hit Compounds

Isothermal calorimetry scanning (ITC) can be used to validate binding of titratable hits. ITC measurements can be performed on a Microcal VP-ITC microcalorimeter (MicroCal Inc., Northampton, Mass.). Samples are filtered and degassed for 10 min prior to loading. Experiments are performed with a sample temperature of 25° C. An assay buffer with 1% DMSO added, is used. The protein concentration in the sample cell would is approximately 10 micromolar. The titration is performed by controlled injections of approximately 200 micromolar compound into the sample cell, allowing 400 sec between injections. The peaks produced over the course of the titration are integrated and used to obtain a plot of the enthalpy change versus the molar ratio of species in the cell. A control experiment is performed to determine the contribution to the binding enthalpy from the heat of dilution of the compound into buffer. The net enthalpy for the interaction between compound and protein is determined by subtraction of the heat of dilution component. Curve fitting is performed using the ORIGIN software to determine the dissociation constants and the number of binding sites for the interaction between compound and protein.

### Example 13

### Characterization of the D56 Target Protein

Recombinant D56, containing a 6xHis tag on the C-terminus, was produced in E. coli and purified using standard methods.

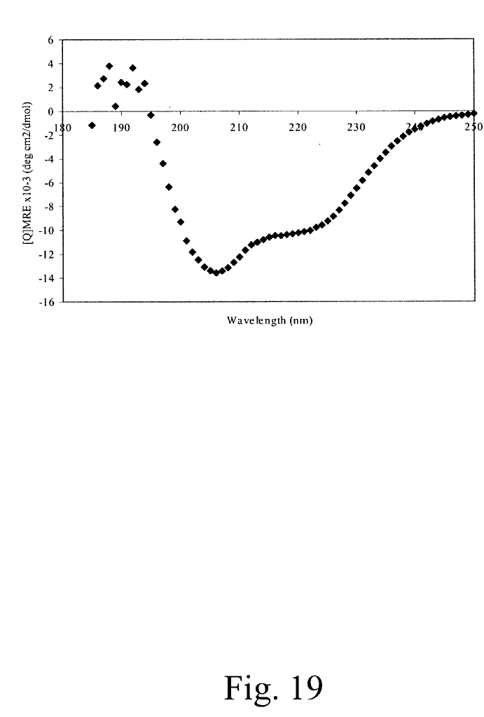

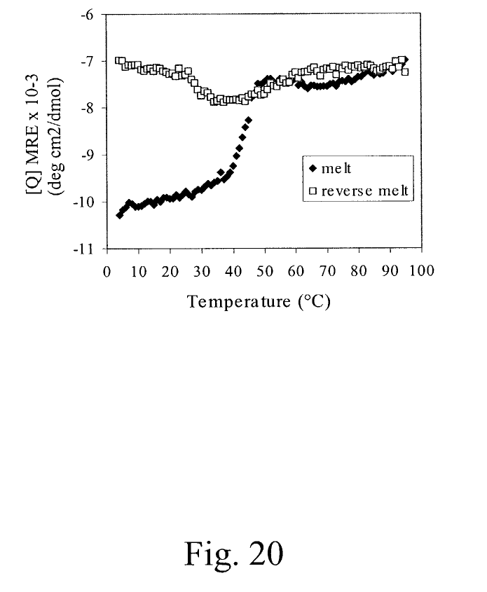

The CD spectra (FIG. 19) of D56 was measured using 0.03 mg/ml protein in 0.2 ml of phosphate buffered saline, using a Circular Dichroism Spectrophotometer (Model 62ADS, Manufacturer: AVIV, Lakewood N.J.). Thermal melting of this sample was performed by heating at a rate of 1° C./minute up to greater than or about 90 degrees C. The ellipticity was measured as a function of temperature to monitor protein unfolding (FIG. 20).

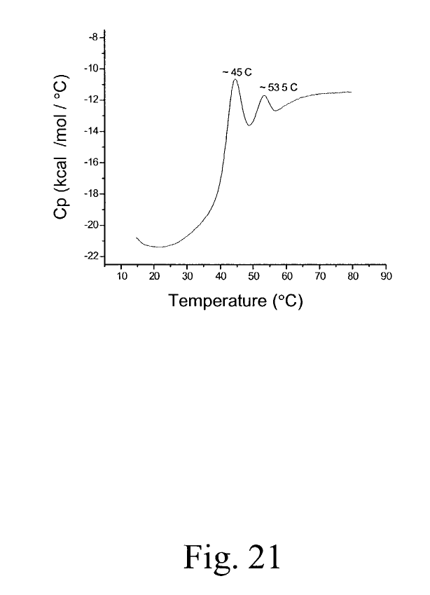

DSC measurements were performed on a Microcal VP-DSC microcalorimeter (MicroCal Inc., Northampton, Mass.). All samples were filtered and degassed for 10 min prior to loading. Samples contained 1.33 mg/ml protein in phosphate buffered saline and were heated at 1° C./min over a temperature range of 15° C.-80° C. DSC measurements for buffer alone were subtracted from the first protein upscan. Data were then normalized and baseline corrected using the Origin DSC software. Differential scanning calorimetry demonstrated that the protein undergoes two transitions (at about 45 degrees C. and at about 53.5 degrees C.) as the temperature is increased (FIG. 21).

### Example 14

### D56 Target Protein Assay Development

ATLAS Mix was made containing 20 mM Na phosphate, pH 7.0, 5% glycerol, 150 mM NaCl, 0.005% Tween 20, 1 mM DTT, 2 nanomolar of D56 directly labeled with FITC, and variable concentrations of unlabeled D56. To configure the assay, thermal melting curves were generated using variable concentrations of unlabeled D56 protein. Each assay mixture was used for 3 sample wells of a 384 well assay plate, with each sample well containing 20 microliters. The plates were placed in MWG PrimusHT Thermocyclers, the lids were closed and heated to 70 degrees C. The thermocyclers were programmed to heat to incubation temperatures ranging from 25 C. to 56 C. at a rate of 2 C. per second, and then to hold at temperature for 30 minutes. The temperature then decreased to 22 C. at a rate of 2 C. per second. The cycler lids were opened and the plates were read using a Victor V plate reader. The plates were read in FP (fluorescence polarization) with an excitation wavelength of 485 nm, and an emission wavelength of 535 nm. The FP value was plotted as a function of temperature (FIG. 22).

FIG. 22 shows FP units as a function of unlabeled protein concentration. The concentration of the trace amount of labeled protein (2 nM) was held constant and did not give an increased signal by itself at higher temperature. Increasing concentrations of unlabeled protein gave better signals at lower transition temperatures.

### Example 15

### D56 Target Protein Assay Validation

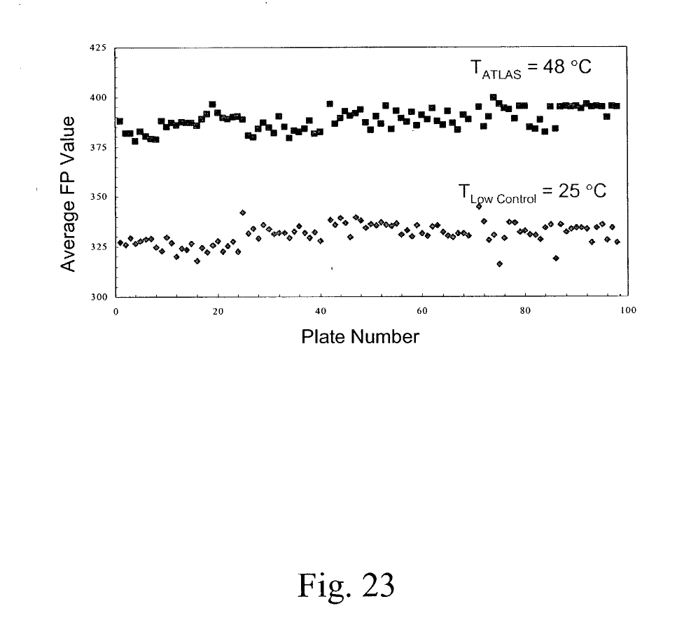

For validation of the D56 FP assay, 4933 compounds were screened in duplicate and 5394 compounds were screened a single time as described in Example 14, using T_{ATLAS}=48 degrees C. and 540 ng per well of target protein. The FP values for the control wells (those having no test compound) from these screening plates, as well as those from several pre-validation plates used in assay development that were screened at both at the selected assay temperature (T_{ATLAS}=48 degrees C.) and at a control temperature (T_{LowControl}=25 degrees C.), are plotted in FIG. 23.

### Example 16

### High Throughput Screen for Ligands of D56

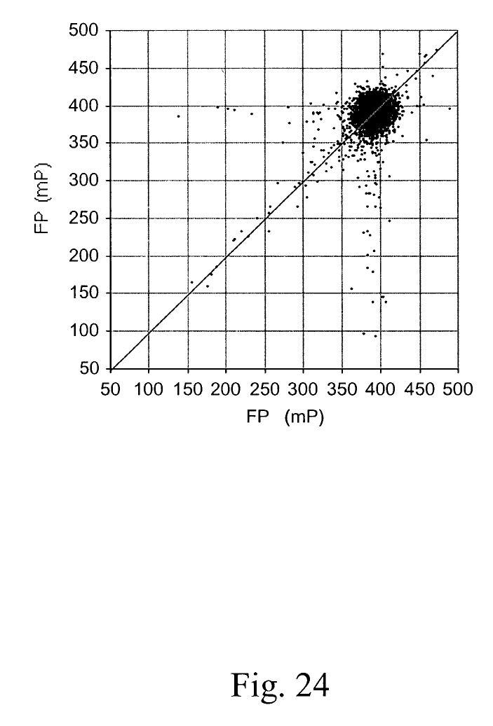

4933 compounds were screened twice for target D56 using T_{ATLAS}=48° C. and 540 ng per well of target protein. For each compound the value of the fluorescence polarization (FP) observed for the assay was plotted; the results from the two screens are plotted against each other in FIG. 24.

### Example 17

### Titration of Duplicate Hits

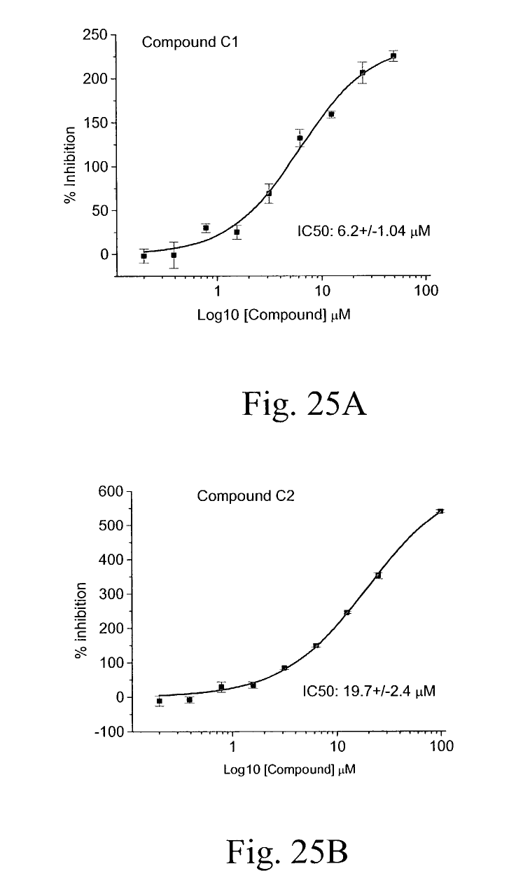

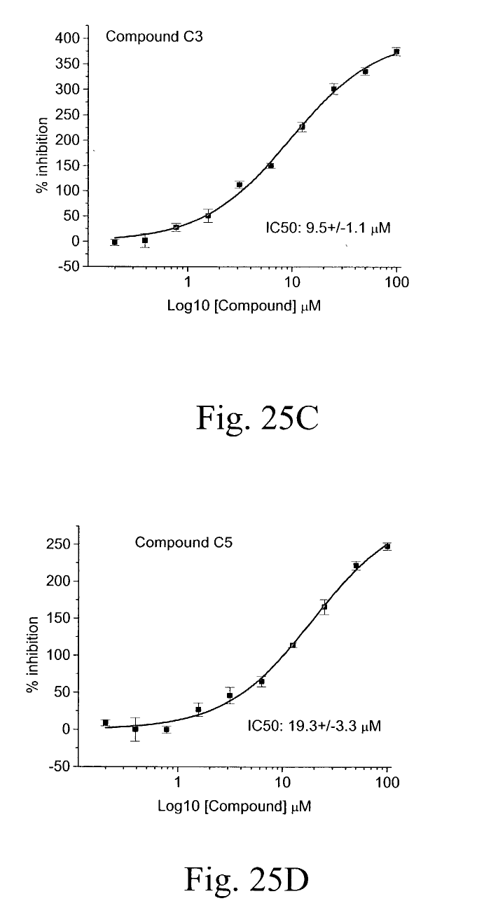

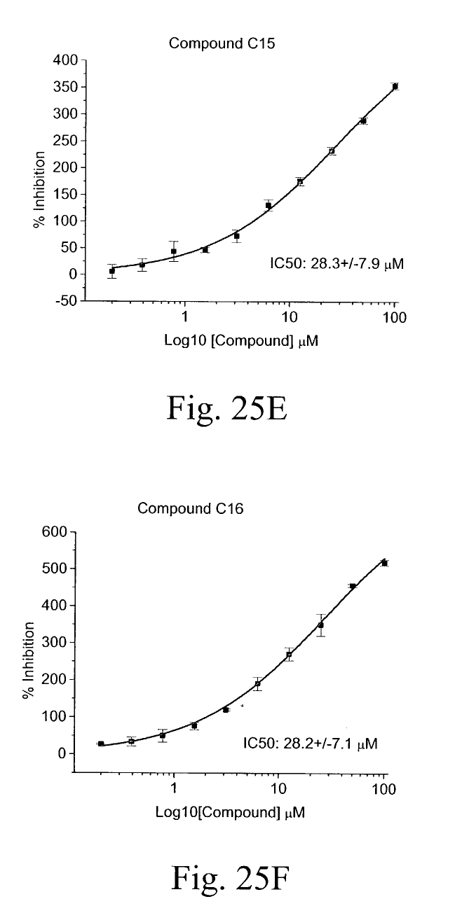

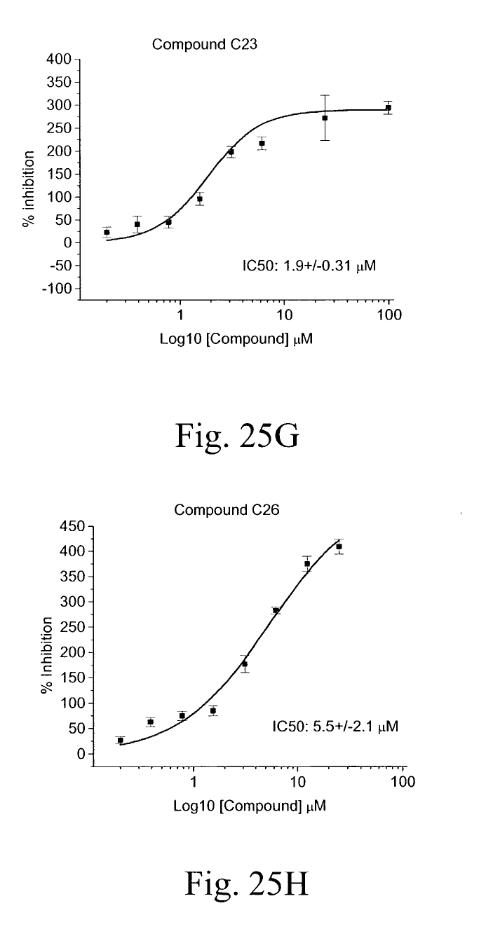

Duplicate hit compound concentrations were titrated by performing a series of 2-fold serial dilutions, creating a series of 11 concentrations for each compound; the highest concentration was 100 uM. These compounds were assayed using the configuration described in Example 14, above; T_{ATLAS}=48° C. and 540 ng per well of target protein. The titration curves of eight of the duplicated hits are shown in FIG. 25.

### Example 18

### Independent Validation of Target Binding

ITC measurements can be performed on a Microcal VP-ITC microcalorimeter (MicroCal Inc., Northampton, Mass.). Samples are filtered and degassed for 10 min prior to loading. Experiments are performed with a sample temperature of 25° C. Assay buffer with 1% DMSO added is used. The protein concentration in the sample cell is approximately 10 micromolar. The titration is performed by controlled injections of approximately 200 micromolar compound into the sample cell, allowing 400 sec between injections. The peaks produced over the course of the titration are integrated and used to obtain a plot of the enthalpy change versus the molar ratio of species in the cell. A control experiment is performed to determine the contribution to the binding enthalpy from the heat of dilution of the compound into buffer. The net enthalpy for the interaction between compound and protein is determined by subtraction of the heat of dilution component. Curve fitting is performed using the ORIGIN software to determine the dissociation constants and the number of binding sites for the interaction between compound and protein.

All publications, including patent documents and scientific articles, referred to in this application, including any bibliography, are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A method of screening to identify one or more ligands that bind to a target molecule, comprising the steps:
(a) providing a target molecule in solution in one or more wells;
(b) adding to said one or more wells one or more test compounds to provide one or more test wells comprising a target molecule and one or more test compounds;
(c) adding to said one or more test wells a first specific binding member that specifically binds the unfolded form of said target molecule, wherein said first specific binding member comprises a FRET donor or a FRET acceptor or can directly or indirectly bind a FRET donor or a FRET acceptor;
(d) subjecting said one or more test wells to conditions at which at least a portion of said target molecule is denatured;
(e) adding to said one or more test wells a second specific binding member that can bind said target protein at a site distinct from the binding site of said first specific binding member, wherein:
when said first specific binding member comprises or can directly or indirectly bind a FRET donor fluorophore, said second specific binding member comprises or can directly or indirectly bind a FRET acceptor, and when said first specific binding member comprises or can directly or indirectly bind a FRET acceptor, said second specific binding member comprises or can directly or indirectly bind a FRET donor;
(f) measuring fluorescence emission at one or more wavelengths from said test wells;
(g) making a comparison of fluorescence emission at one or more wavelengths of said one or more test wells with one or more reference values;
(h) using said comparison in step (g) to determine the extent to which said target molecule occurs in the unfolded state, the folded state, or both in said one or more test wells; and
(i) using the determination in part (h) to determine whether said one or more test compounds binds said target molecule, thereby identifying one or more ligands of said target molecule.

2. The method of claim 1, wherein said subjecting said one or more test wells to conditions at which at least a portion of said target molecule is denatured comprises heating said one or more test wells to one or more predetermined temperatures at which at least a portion of said target molecule is denatured.

3. The method of claim 2, wherein said at least one predetermined temperature is one predetermined temperature.

4. The method of claim 1, wherein said target molecule is a target protein.

5. The method of claim 4, wherein said target protein comprises an attached tag that is recognized by said second specific binding member

6. The method of claim 5, wherein said attached tag is a chemical moiety.

7. The method of claim 6, wherein said chemical moiety is DNP or biotin.

8. The method of claim 5, wherein said attached tag is an engineered peptide tag.

9. The method of claim 8, wherein said engineered peptide tag is a 6xHis tag, a FLAG tag, a myc tag, or a hemaglutinin tag.

10. The method of claim 1, wherein said first specific binding member is an antibody that specifically binds the unfolded form of said target protein.

11. The method of claim 10, wherein said antibody can directly or indirectly bind a FRET donor or a FRET acceptor.

12. The method of claim 11, wherein said antibody can indirectly bind a FRET donor or a FRET acceptor.

13. The method of claim 12, wherein said antibody is bound to biotin, and said FRET donor or FRET acceptor is bound to streptavidin.

14. The method of claim 13, wherein step (e) further comprises adding said FRET donor or FRET acceptor bound to streptavidin.

15. The method of claim 12, wherein said second specific binding member is an antibody.

16. The method of claim 14, wherein said second specific binding member is an antibody that is directly bound to a FRET donor or FRET acceptor.

17. The method of claim 1, wherein said FRET donor is terbium, Alexa 488, Alexa 568, Alexa 594, Alexa 647, Cy3, BODIPY FL, fluorescein, IEDANS, EDANS, or Europium cryptate.

18. The method of claim 20, wherein said FRET donor is Europium cryptate.

19. The method of claim 1, wherein said FRET acceptor is fluorescein, GFP, TMR, Cy3, R phycoerythrin, Cy5, APC, Alexa 555, Alexa 647, Alexa 647, Alexa 594, Cy5, BODIPY FL, TMR, DABCYL, XL-665, or allophycocyanin.

20. The method of claim 19, wherein said FRET acceptor is XL-665.

21. The method of claim 1, wherein said one or more wavelengths is two wavelengths.

22. The method of claim 3, further comprising expressing said fluorescence emission as a ratio of fluorescence emission at two wavelengths.

23. The method of claim 1, wherein said reference value is one or more measurements or calculated values from one or more control wells.

24. The method of claim 1, wherein said reference value is one or more measurements or calculated values from one or more standard wells.

25. A method of screening to identify one or more ligands that bind to a target molecule, comprising the steps:
(a) providing a population of a target molecule, wherein at least a portion of said population is labeled with a first specific binding member, wherein said first specific binding member can bind a single attached tag of said target molecule and wherein said first specific binding member comprises or can directly or indirectly bind a FRET donor or a FRET acceptor;
(b) contacting an aliquot of said population of a target molecule with at least one test compound in one or more test wells;
(c) subjecting said one or more test wells to conditions at which at least a portion of said target protein is denatured;
(d) adding to said one or more test wells a second specific binding member that binds said single attached tag of said target molecule, wherein said second specific binding member comprises or can bind an acceptor or donor fluorophore, wherein:
when said first specific binding member comprises or can directly or indirectly bind a FRET donor, said second specific binding member comprises or can directly or indirectly bind a FRET acceptor, and when said first specific binding member comprises or can directly or indirectly bind a FRET acceptor, said second specific binding member comprises or can directly or indirectly bind a FRET donor;
(e) measuring fluorescence emission at one or more wavelengths from said one or more test wells;
(f) comparing fluorescence emission at said one or more wavelengths of said one or more test wells with one or more reference values;
(g) determining the extent to which said target molecule occurs in the unfolded state, the folded state, or both in said one or more test wells; and
(h) using the determination in part (g) to determine whether one or more test compounds binds said target molecule, thereby identifying one or more ligands of said target molecule.

26. The method of claim 25, wherein said at least a portion is approximately 50% of said population.

27. The method of claim 25, wherein said at least a portion is at least 80% of said population.

28. The method of claim 25, wherein said subjecting said one or more test wells to conditions at which at least a portion of said target molecule is denatured comprises heating said one or more test wells to one or more predetermined temperatures at which at least a portion of said target molecule is denatured.

29. The method of claim 28, wherein said at least one predetermined temperature is one predetermined temperature.

30. The method of claim 25, wherein said target molecule is a target protein that comprises a single attached tag.

31. The method of claim 30, wherein said single attached tag is a chemical moiety.

32. The method of claim 31, wherein said chemical moiety is DNP or biotin.

33. The method of claim 30, wherein said single attached tag is an engineered peptide tag.

34. The method of claim 33, wherein said engineered peptide tag is a 6xHis tag, a FLAG tag, a myc tag, or a hemaglutinin tag.

35. The method of claim 25, wherein said first specific binding member is an antibody.

36. The method of claim 35, wherein said antibody comprises or can directly or indirectly bind a donor fluorophore.

37. The method of claim 36, wherein said antibody comprises a donor fluorophore.

38. The method of claim 37, wherein said donor fluorophore is, terbium, Alexa 488 , Alexa 568, Alexa 594, Alexa 647, Cy3, BODIPY FL, fluorescein, IEDANS, EDANS, or Europium.

39. The method of claim 38, wherein said second specific binding member comprises or can directly or indirectly bind an acceptor fluorophore.

40. The method of claim 39, wherein said second specific binding member comprises an acceptor fluorophore.

41. The method of claim 40, wherein said acceptor fluorophore is fluorescein, GFP, TMR, Cy3, R phycoerythrin, Cy5, APC, Alexa 555, Alexa 647, Alexa 647, Alexa 594, Cy5, BODIPY FL, TMR, DABCYL, or XL665.

42. The method of claim 25, wherein in steps (e) and (f), said one or more wavelengths is two wavelengths.

43. The method of claim 42, further comprising, after step (e), calculating a ratio between fluorescence emission at said two wavelengths from said one or more test wells, and wherein in step (f), comparing fluorescence emission at two wavelengths comprises comparing a ratio of fluorescence emission at two wavelengths.

44. The method of claim 43, wherein said one or more reference values is one or more ratios between fluorescence emission at said two wavelengths from one or more control wells.

45. The method of claim 43, wherein said one or more reference values is one or more ratios between fluorescence emission at said two wavelengths from one or more standard wells.

46. A method of screening to identify one or more ligands that bind to a target molecule, comprising the steps:
(a) providing a first population of a target molecule that comprises or can bind a FRET donor or a FRET acceptor;
(b) adding to said first population of said target molecule a second population of said target molecule that comprises or can bind a FRET donor or a FRET acceptor, to generate a mixed donor/acceptor population of said target molecule, wherein:
when said first specific binding member comprises or can directly or indirectly bind a FRET donor, said second specific binding member comprises or can directly or indirectly bind a FRET acceptor, and when said first specific binding member comprises or can directly or indirectly bind a FRET acceptor, said second specific binding member comprises or can directly or indirectly bind a FRET donor;
(c) contacting an aliquot of said mixed donor/acceptor population of said target molecule with at least one test compound in one or more test wells;
(d) subjecting said one or more test wells to conditions at which at least a portion of said target protein is denatured;
(e) measuring fluorescence emission at one or more wavelengths from said one or more test wells;
(f) comparing fluorescence emission at one or more wavelengths of said one or more test wells with one or more reference values;
(g) determining the extent to which the target molecule occurs in the unfolded state, the folded state, or both, in said one or more test wells; and
(h) using the determination in part (g) to determine whether one or more test compounds binds said target molecule, thereby identifying one or more ligands of said target molecule.

47. The method of claim 46, wherein said subjecting said one or more test wells to conditions at which at least a portion of said target molecule is denatured comprises heating said one or more test wells to one or more predetermined temperatures.

48. The method of claim 47, wherein said at least one predetermined temperature is one predetermined temperature.

49. The method of claim 46, wherein said target molecule is a target protein.

50. The method of claim 49, wherein said target protein comprises an attached tag.

51. The method of claim 50, wherein said attached tag is a chemical moiety.

52. The method of claim 51, wherein said chemical moiety is DNP or biotin.

53. The method of claim 50, wherein said attached tag is an engineered peptide tag.

54. The method of claim 53, wherein said engineered peptide tag is a 6xHis tag, a FLAG tag, a myc tag, or a hemaglutinin tag.

55. The method of claim 46, wherein said FRET donor or said FRET acceptor is directly bound to said first population of said target protein.

56. The method of claim 53, wherein said FRET donor or said FRET acceptor is indirectly bound to said first population of said target protein.

57. The method of claim 55, wherein said FRET donor or said FRET acceptor is bound to said first population of said target protein via a specific binding member that recognizes said engineered peptide tag of said target protein.

58. The method of claim 53, wherein said FRET donor or FRET acceptor is indirectly bound to said second population of said target protein.

59. The method of claim 58, wherein said FRET donor or FRET acceptor is bound to said second population of said target protein via a specific binding member that recognizes said engineered peptide tag of said target protein.

60. The method of claim 46, wherein in steps (e) and (f), said one or more wavelengths is two wavelengths.

61. The method of claim 46, further comprising, after step (e), calculating a ratio between fluorescence emission at said two wavelengths from said one or more test wells, and wherein in step (f), comparing fluorescence emission at two wavelengths comprises comparing a ratio of fluorescence emission at two wavelengths.

62. The method of claim 61, wherein said reference value comprises at least one ratio between fluorescence emission at said two wavelengths.

63. The method of claim 62, wherein said one or more reference values is one or more ratios between fluorescence emission at said two wavelengths from one or more control wells.

64. The method of claim 62, wherein said one or more reference values is one or more ratios between fluorescence emission at said two wavelengths from one or more standard wells.

65. A method of screening to identify one or more ligands that binds to a target molecule, comprising the steps:
(a) labeling at least a portion of a population of a target molecule with at least one fluorophore;
(b) dispensing aliquots of said population of said target molecule in one or more test wells;
(c) adding to said one or more test wells one or more test compounds;
(d) subjecting said one or more test wells to conditions at which at least a portion of said target protein is denatured;
(e) measuring fluorescence polarization from said one or more test wells;
(f) comparing said fluourescence polarization measurements from said one or more test wells with a reference value;
(g) determining the extent to which the target molecule occurs in the unfolded state, the unfolded state, or both in the plurality of test wells and in said one or more control wells or control values; and
(h) using the determination in part (g) to determine whether one or more test compounds binds said target molecule, thereby identifying one or more ligands of said target molecule.

66. The method of claim 65, wherein said subjecting said one or more test wells to conditions at which at least a portion of said target molecule is denatured comprises heating said one or more test wells to one or more predetermined temperatures.

67. The method of claim 66, wherein said at least one predetermined temperature is one predetermined temperature.

68. The method of claim 65, wherein said target molecule is a target protein.

69. The method of claim 68, wherein said target protein is indirectly bound to a fluorophore.

70. The method of claim 68, wherein said target protein is directly bound to a fluorophore.

71. The method of claim 65, wherein said reference value is an average of fluorescence polarization measurements from two or more control wells.

72. The method of claim 65, wherein said reference value is an average of fluorescence polarization measurements from two or more standard wells.

73. A method of screening to identify one or more ligands that bind to a target molecule comprising the steps:
(a) providing a target molecule in solution in one or more test wells;
(b) adding to said one or more test wells one or more test compounds;
(c) adding to said one or more test wells at least one specific binding member that specifically binds the unfolded form of said target molecule, wherein said at least one first specific binding member comprises a fluorophore or can directly or indirectly bind a fluorophore;
(d) subjecting said one or more test wells conditions at which at least a portion of said target molecule is denatured;
(e) measuring fluorescence polarization from said one or more test wells;
(f) comparing said fluourescence polarization from said one or more test wells with a reference value;
(g) determining the extent to which said target molecule occurs in the unfolded state, the folded state, or both; and
(h) using the determination in part (g) to determine whether one or more test compounds binds said target molecule, thereby identifying one or more ligands of said target molecule.

74. The method of claim 70, wherein said subjecting said one or more test wells to conditions at which at least a portion of said target molecule is denatured comprises heating said one or more test wells to one or more predetermined temperatures.

75. The method of claim 71, wherein said at least one predetermined temperature is one predetermined temperature.

76. The method of claim 70, wherein said target molecule is a target protein.

77. The method of claim 70, wherein said specific binding member is indirectly bound to a fluorophore.

78. The method of claim 70, wherein said specific binding member is directly bound to a fluorophore.

79. The method of claim 78, wherein said specific binding member is coupled to a bead or particle.

80. A method of screening to identify one or more ligands that binds to a target molecule, comprising the steps:
(a) labeling at least a portion of a population of a target molecule with at least one fluorophore;
(b) dispensing aliquots of said population of said target molecule in one or more test wells;
(c) adding to said one or more test wells one or more test compounds;
(d) adding to said one or more test wells at least one specific binding member that specifically binds the unfolded form of said target molecule;
(e) subjecting said one or more test to conditions at which at least a portion of said target protein is denatured;
(f) measuring fluorescence polarization from said one or more test wells;
(g) comparing said fluourescence polarization from said one or more test wells with a reference value;
(h) determining the extent to which the target molecule occurs in the unfolded state, the folded state, or both in said one or more test; and
(i) using the determination in part (h) to determine whether one or more test compounds binds said target molecule, thereby identifying one or more ligands of said target molecule.

81. The method of claim 80, wherein said subjecting said one or more test wells to conditions at which at least a portion of said target molecule is denatured comprises heating said one or more test wells to one or more predetermined temperatures.

82. The method of claim 81, wherein said at least one predetermined temperature is one predetermined temperature.

83. The method of claim 80, wherein said target molecule is a target protein.

84. The method of claim 80, wherein said target molecule is indirectly bound to a fluorophore.

85. The method of claim 80, wherein said target molecule is directly bound to a fluorophore.

86. The method of claim 80, wherein said at least one specific binding member is coupled to a bead or particle.

87. The method of claim 80, wherein at least one specific binding member is at least two specific binding members.

88. The method of claim 87, wherein said at least two specific binding members comprises at least one primary antibody and at least one secondary antibody.
